(19) United States
(12) Patent Application Publication
(10) Pub. No.: US 2024/0384328 A1
(43) Pub. Date: Nov. 21, 2024

(54) ANALYTIC PLATFORM USING NPM1-ASSOCIATED GENES INTERACTION NETWORK FOR IDENTIFYING GENETIC TRAITS

(71) Applicant: B.Y. QUANTITATIVE MEDICINE LIMITED, Hong Kong (CN)

(72) Inventor: Yat-ming YUNG, Hong Kong (CN)

(21) Appl. No.: 18/692,344

(22) PCT Filed: Feb. 9, 2023
(86) PCT No.: PCT/IB2023/051145

§ 371 Date: Mar. 15, 2024

Related U.S. Application Data
(60) Provisional application No. 63/308,067, filed on Feb. 9, 2022.

(51) Int. Cl. C12Q 1/6809; G16B 25/10

(52) U.S. Cl. CPC C12Q 1/6809; G16B 25/10

(57) ABSTRACT

This invention provides a method for identifying a genetic trait of cells in a state of interest, a computer-implemented method for identifying a genetic trait of cells in a state of interest, a non-transitory computer-readable medium having stored thereon program instructions that, upon execution by a computing device, cause the computing device to perform operations for identifying a genetic trait of cells in a state of interest and a computing device comprising: 1) a processor; 2) memory; and 3) program instructions, stored in the memory, that upon execution by the processor cause the computing device to perform operations for identifying a genetic trait of cells in a state of interest.

### FIELD OF THE INVENTION

The present invention relates to platforms for analyzing gene co-expression/interaction so as to identify genetic traits.

### BACKGROUND OF THE INVENTION

Nucleophosmin 1 (NPM1/B23) is a multifunctional nucleolar protein found in proliferating cells and is involved in ribosome biogenesis, genomic stability, DNA repair, cell cycle, and apoptosis. It is an important nucleolar phosphoprotein involved in the regulation of assorted cellular signaling pathways. It has been described as chromatin associated proteins with histone chaperone activities and also as proteins able to regulate chromatin transcription. It is to be over-expressed in highly proliferative cells and is involved in many aspects of gene expression: chromatin remodeling, DNA recombination and replication, RNA transcription by RNA polymerase I and II, rRNA processing, mRNA stabilization, cytokinesis, and apoptosis. NPM1 is also found on the cell surface in a wide range of cancer cells, a property which is being used as a marker for the diagnosis of cancer and for the development of anti-cancer drugs to inhibit the proliferation of cancer cells.

In a lung adenocarcinoma cell line, forced expression of NPM1 has been shown to increase cell migration and invasion in a dose-dependent manner (Chang et al., 2010).

In another cell line, the oncogenic or tumor suppressive property of NPM1 relies on the identity of its binding partner. Human liver Dna-J like protein (HLJ1) belongs to the heat shock protein 40 family of chaperones (Chang et al., 2010).

It is a tumor suppressor shown to attenuate metastasis in non-small cell lung cancer. HLJ1 binds competitively to NPM1 and impairs NPM1 oligomerization and nuclear distribution (Chang et al., 2010).

NPM1 acts as either oncogenic or tumor suppressive depending on its binding activity with HLJ1 (Chang et al., 2010). HLJ1 binding alters the function of NPM1, allowing the formation of a new complex with activator protein 2 alpha (AP2a), a tumor suppressor. The trio complex acts as a co-repressor and downregulates AP2a-regulated genes such as matrix metalloproteinase-2 (MMP-2), impeding cell migration and invasion (Chang et al., 2010). Silencing HLJ1 and enforcing the expression of NPM1 increases the phosphorylation of signal transducer and activator of transcription 3 (STAT3) and the expression of MMP-2, which ultimately promotes oncogenesis (Chang et al., 2010).

Another binding partner of NPM1 is c-Myc. c-Myc is a transcription factor essential to the regulation of cell proliferation and transformation (Li et al., 2008). NPM1 can bind to the transcriptional regulatory domains of c-Myc at the N-terminal Myc Box II (MBII) domain and the C-terminal helix-loop-helix-leucine-zipper domain and exert transcriptional control over c-Myc target genes (Li et al., 2008).

Elevated expression of NPM1 in solid tumors is associated with disease progression. In colon carcinoma, metastatic lymph nodes have higher NPM1 expression and are associated with shorter survival (Liu et al., 2012). Tissue staining shows that there is significantly more NPM1 in cancer tissue compared to adjacent normal and NPM1 is also found more frequently in invasive than weakly invasive cancer cells (Liu et al., 2012). This concurs with the finding that NPM1 downregulation impairs cell proliferation, migration, and Literature review 22 invasions, while upregulation enhances cell invasiveness (Liu et al., 2012). Similarly, in bladder cancer, high NPM1 expression is associated with advanced tumor stage and grade, poor prognosis, and higher risk of recurrence (Tsui et al., 2008). Forced NPM1 expression in lung cancer cells also increases cell invasiveness and migratory potential, while the impairment of NPM1 oligomerization weakens malignancy. NPM1 overexpression restores oligomerization and its associated cancerous phenotype (Chang et al., 2010).

The localization of NPM1 is linked to drug sensitivity (Cilloni et al., 2008). In AML patients, the presence of cytoplasmic NPM1 enhances cellular chemosensitivity (Cilloni et al., 2008). In the cytoplasm, NPM1 is shown to sequestrate and inactivate cytoplasmic NF-κB, which is known to induce chemoresistance (Cilloni et al., 2008). In thyroid tumor cells, NPM1 is found localized in the cytoplasm, nucleus, and nucleolus, but only in the nucleolus in non-tumorigenic thyroid cells (Pianta et al., 2011). Furthermore, inducing differentiation in hepatocarcinoma cells delocalizes NPM1 from the nuclear matrix to the nucleoplasm, nuclear membrane and cytoplasm (Li et al., 2020b). Evidently, NPM1 localization is associated with cancer development and drug response.

The development of Next-generation sequencing (NGS) is a massively parallel sequencing technology that offers ultra-high throughput, scalability, and speed. The technology is used to determine the order of nucleotides in entire genomes or targeted regions of DNA or RNA, granting researchers an opportunity to explore genome-wide co-expression networks. Differential co-expression analysis identifies genetic perturbations between disease and healthy samples and provides mechanistic information on disease-affected regulatory networks (Kostka and Spang, 2004). Co-expression analysis is used to understand and develop prognostic value in various diseases including cancer (Wu et al., 2019), diabetes (Riquelme Medina and Lubovac-Pilav, 2016), obesity (Wang et al., 2017), depression (Wang et al., 2019b), Alzheimer's disease (Tang and Liu, 2019), organ injury (Wang et al., 2019c), and parasitic infection (Siwo et al., 2015).

Transcription factors are proteins with DNA binding properties and take part in transcription initiation and elongation (Lee and Young, 2013). Transcription factors function by binding to the enhancer elements of their target genes, which triggers a loop formation bringing the enhancer element closer to the promoter of nearby or distant genes (Lee and Young, 2013). The binding of transcription factors also recruits activating (coactivators) or repressing (corepressors) cofactors and RNA polymerase II to the initiation site (Lee and Young, 2013). Cofactors can influence transcription rate by altering chromatin structure and thereby its accessibility (Lee and Young, 2013). c-Myc is one of the most widely studied transcription factors and is known as a master regulator and driver of malignant transformation (Miller et al., 2012a). It controls transcription by stimulating the release of RNA polymerase II from its pause site after initial transcription initiation (Lee and Young, 2013).

MicroRNAs (miRNAs) are small non-coding regulatory RNA molecules found in animals, plants, and viruses, and work to silence messenger RNA (mRNA) (Flynt and Lai, 2008). They are processed from long hairpin-containing primary transcripts and cleaved to yield a 21-24 nucleotide long mature miRNA (Flynt and Lai, 2008). The mature miRNA together with RNA-induced silencing complex (RISC), a multiprotein complex, bind to complementary mRNA at the 3′ untranslated region (3′ UTR) and activate either mRNA degradation or translational repression (Flynt and Lai, 2008). An individual miRNA can target hundreds to thousands of mRNA with as few as seven complementary nucleotides needed, while one mRNA molecule can be suppressively targeted by multiple different miRNAs (Flynt and Lai, 2008, Lin and Gregory, 2015).

### SUMMARY OF THE INVENTION

This invention provides a method for identifying a genetic trait of cells in a state of interest. In one embodiment, said method comprises the steps of: a) Obtaining a first gene expression data from cells in said state of interest; b) Obtaining a second gene expression data from cells in a reference state; c) Conducting one or both of the following steps: 1) Identifying a first set of target genes, wherein each gene in said first set of target genes is strongly co-expressed with another gene in said first set of target genes in said state of interest as compared to said reference state by: i) Conducting a first co-expression analysis on said first gene expression data to arrive at a first co-expression data; ii) Conducting a second co-expression analysis on said second gene expression data to arrive at a second co-expression data; iii) Comparing said first and second co-expression data to identify said first set of target genes; 2) Identifying a second set of target genes, wherein each target gene in said second set of target genes are differentially expressed genes with high connectivity in said state of interest as compared to said reference state by: i) Conducting differential expression analysis on said first gene expression data to identify a set of differentially expressed genes in said state of interest with respect to said reference state; ii) Identify said second set of target genes with high connectivity among said set of differentially expressed genes; d) Identifying a third set of target genes, wherein each target gene in said third set of target genes is strongly co-expressed with NPM1 in said state of interest as compared to said reference state; e) Conducting functional enrichment or pathway enrichment on said target genes obtained from steps (c) to (d); f) Identifying signaling pathways associated with said target genes; and g) Comparing said signaling pathways against a database to identify said genetic trait.

This invention also provides a computer-implemented method for identifying a genetic trait of cells in a state of interest.

This invention also provides a non-transitory computer-readable medium having stored thereon program instructions that, upon execution by a computing device, cause the computing device to perform operations for identifying a genetic trait of cells in a state of interest.

This invention further provides a computing device comprising: 1) a processor; 2) memory; and 3) program instructions, stored in the memory, that upon execution by the processor cause the computing device to perform operations for identifying a genetic trait of cells in a state of interest.

### BRIEF DESCRIPTION OF THE FIGURES

FIG. 1 shows the gene expression data analysis process for HER2-Positive Breast Cancer Relapse in one embodiment of this invention. Dataset (GSE55348) was retrieved from GEO data repository and analyzed separately in two ways (1) differential gene expression analysis, and (2) whole genome co-expression analysis. All gene sets derived were subjected to functional enrichment. Differential expression was used to understand pathway disruption leading to patient relapse. Co-expression analysis was used to identify disruption in ERBB2 and NPM1 co-expression network. Construction of Standard Curve of Co-Expression.

FIG. 2 shows the gene expression data analysis process for Ovarian Cancer in one embodiment of this invention. Dataset GSE51373 was retrieved from Gene Expression Omnibus (GEO) database and was analyzed in gene-gene co-expression analysis. The chemoresistance-specific genes were subjected to pathway enrichment analysis on ClueGo. Gene gene co-expressed module were developed through literature review and validated by using dataset GSE131978. The interconnected pathways were identified by literature search and construction of Standard Curve of Co-Expression.

FIG. 3 Heatmap of co-expressed genes in relapsed state were generated while the gene with no relevant evidence relating it to humoral immune (HPX) were not included. The pattern of gene expression level relating to NPM1 expression level was less distinct in humoral immune response mediated by circulating immunoglobulin than in DNA repair. By analyzing the genes one by one, expression levels CXCL10 and IGLL1 (with p=0.031 and 0.02) in relapsed patients were also significantly lower than that of non-relapsed patients when NPM1 expression was lower than the threshold (using the mean of NPM1 expression level of 53 patients). In high NPM1 expression, three negatively correlated genes, CXCL10, MASP2 and IGLL1 (with p=0.042, 0.06 and 0.046 respectively), were significantly lower in relapsed patients than that in non-relapsed patients when NPM1 expression was higher than the threshold (using the mean of NPM1 expression level of 53 patients).

FIGS. 4a, 4b, 4c and 4d show the heatmap of co-expressed genes in non-relapsed state were generated while the four genes with no relevant evidence of DNA repair were not included. From the heatmap, when NPM1 expression level was low, the expression levels of positively correlated genes were low in both non-relapsed and relapsed patients. However, when NPM1 expression level was high, the expression levels of positively correlated genes were higher in non-relapsed patients than that of relapsed patients (with p=0.003, using the mean of NPM1 expression level of 53 patients as threshold level). By analyzing the genes one by one, expression levels of 15 genes, CDCA5, CDK1, COPS7B, EXO1, FANCB, FANCD2, GINS4, MEN1, MMS22L, NTHL1, PARP2, POLR2D, RFC2, RNASEH2A and UBE2L6 (with p=0.015, 0.007, 0.009, 0.03, 0.003, 0.005, 0.049, 0.044, 0.049, 0.04, 0.038, 0.015, 0.006, 0.003 and 0.039), were significantly higher in non-relapsed patients than in relapsed patients when NPM1 was high. For negative correlated genes, the expression levels were not significantly correlated with NPM1 expression between non-relapsed and relapsed patients.

FIG. 5 shows the microscopic view of ERBB2, NPM1, IFNG, STAT1, HLADQB2, B2M, FCGR1A, TRIM62, PTAFR, and VCAM1 in interferon-gamma-mediated signaling.

FIG. 6 shows the simplified network of complement cascade. The perturbations of complement cascade may involve the malfunctioning of phagocytosis, inflammation, membrane attack complex and Breg activation. The filled oval boxes represented chemoresistance-specific genes while the oval hollow boxes represented non-chemoresistance-specific genes in the co-expression analysis.

FIG. 7 shows the proposed interconnected network under chemoresistance state of high-grade serous ovarian cancer (HGSOC). The filled rectangular boxes represented chemoresistance-specific genes while the oval hollow boxes were the non-chemoresistance-specific genes in co-expression analysis.

FIG. 8 shows the Complement Cascade Heatmap.

FIG. 9 shows the Epithelial-mesenchymal transition (EMT) Heatmap.

FIG. 10 shows the Adaptive Immunity Heatmap.

FIG. 11 shows the JAK/STAT Heatmap.

FIG. 12 shows the PI3K/AKT Heatmap.

FIG. 13 shows the microscopic view of the interconnected network of Complement Cascade, Epithelial-mesenchymal transition (EMT), Adaptive Immunity, JAK/STAT, and PI3K/AKT modules. The filled boxes represent genes in the module.

FIG. 14 shows the macroscopic view of the interconnected network of Complement Cascade, Epithelial-mesenchymal transition (EMT), Adaptive Immunity, JAK/STAT, and PI3K/AKT modules.

FIG. 15 shows the macroscopic view of the interconnected network of the Sensory Development

FIG. 16 shows the macroscopic view of the interconnected network of the Neuron Development

FIG. 17 shows the Sensory Development Heatmap

FIG. 18 shows the microscopic view of the interconnected network of the Sensory Development

FIG. 19 shows the Neuron Development Heatmap

FIG. 20 shows the microscopic view of the interconnected network of the Neuron Development

FIG. 21 shows the Neuroendocrine response Heatmap.

FIG. 22 shows the microscopic view of the interconnected network of the Neuroendocrine response.

FIG. 23 shows the macroscopic view of the interconnected network of the Neuroendocrine response.

FIG. 24 shows the microscopic view of the interconnected network of the Olfactory Receptors.

FIG. 25 shows the Olfactory Receptors Heatmap.

FIG. 26 shows the macroscopic view of the interconnected network of the Olfactory Receptors.

FIG. 27 shows the macroscopic view of the interconnected network of the Tissue Development-Wnt pathway.

FIG. 28 shows the macroscopic view of the interconnected network of the Cellular Response to FGF.

FIG. 29 shows the Tissue Development-Wnt pathway Heatmap.

FIG. 30 shows the microscopic view of the interconnected network of the Tissue Development-Wnt pathway.

FIG. 31 shows the Tissue Development-Hippo pathway Heatmap.

FIG. 32 shows the microscopic view of the interconnected network of the Tissue Development-Hippo pathway.

FIG. 33 shows the macroscopic view of the interconnected network of the Tissue Development-Hippo pathway.

FIG. 34 shows the macroscopic view of the interconnected network of the Cellular Response to Estrogen.

FIG. 35 shows the Tissue Development-TGFB-ITG Pathway Heatmap.

FIG. 36 shows the Tissue Development-TGF-beta pathway Heatmap.

FIG. 37 shows the microscopic view of the interconnected network of the Tissue Development-TGFB-ITG and TGF-beta pathway Pathways.

FIG. 38 shows the Cellular Response to Estrogen Heatmap.

FIG. 39 shows the Cellular Response to FGF Heatmap.

FIG. 40 shows the microscopic view of the interconnected network of the Cellular Response to Estrogen.

FIG. 41 shows the microscopic view of the interconnected network of the Cellular Response to FGF.

FIG. 42 shows the Cellular Response: p53 Pathway Heatmap.

FIG. 43 shows the microscopic view of the interconnected network of the Cellular Response: p53 Pathway.

FIG. 44 shows the macroscopic view of the interconnected network of the Cellular Response: p53 Pathway.

FIG. 45 shows the macroscopic view of the interconnected network of the Cellular Response to Progesterone.

FIG. 46 shows the microscopic view of the interconnected network of the Cellular Response to Progesterone.

FIG. 47 shows the Cellular Response to Progesterone Heatmap.

FIG. 48 shows the Cellular Response to TNF Heatmap.

FIG. 49 shows the microscopic view of the interconnected network of the Cellular Response to TNF.

FIG. 50 shows the macroscopic view of the interconnected network of the Cellular Response to TNF.

FIG. 51 shows the macroscopic view of the interconnected network of the Cellular Response to NGF.

FIG. 52 shows the Cellular Response to NGF Heatmap

FIG. 53 shows the microscopic view of the interconnected network of the Cellular Response to NGF.

FIG. 54 shows the gene expression data analysis process for Colorectal Cancer in one embodiment of this invention. Microarray datasets of colorectal cancer were collected from Gene Expression Omnibus (GEO) database (GEO). After pre-processing, data in GSE17537 as the training set was analyzed by MATLAB for co-expression analysis and by Cystoscope for functional enrichment analysis. The co-expressed genes involved in Wnt signaling pathway were screened out and heatmaps were constructed to show the expression pattern. Kaplan-Meier (KM) survival curves and log rank tests were done to examine the association between gene expression and disease-free survival (DFS) and overall survival (OS). Model was validated by independent dataset GSE17536 and Standard Curve of Co-Expression is constructed.

FIGS. 55a and 55b show the Wnt-recurrence risk model Heatmap. (a) Heatmap of the four gene recurrence risk model arranged according to predicted risk. Patients were stratified into two groups in accordance with predicted risk (low risk and high risk). Samples within the groups were arranged according to NPM1 expression from low to high. (b) Heatmap of the four gene recurrence risk model arranged according to stages. Patients were arranged based on their AJCC stages. Samples within the groups were arranged according to NPM1 expression from low to high.

FIG. 56 shows the microscopic view of the interconnected network of the Wnt-recurrence risk model. Genes involved in Wnt signaling pathway. Genes in circle represent gene co-expressed negatively with NPM1.Genes in hexagon represent genes co-expressed positively with NPM1. Kanahisa 2022, Wnt signalling pathway, diagram by Kanahisa Laboratories, KEGG, accessed January 2023, <https://www.genome.jp/pathway/bsa04310>.

FIG. 57 shows the macroscopic view of the interconnected network of the Wnt-recurrence risk model and summary of the roles of genes contributing to aggressiveness of CRC cells. Genes in rectangular box represents genes upregulated in CRC and genes in hexagon represents genes downregulated in CRC. Figure made in biorender.com.

FIG. 58 shows the heatmap of 95 genes involved in Wnt signaling pathway. Samples were separated into relapse and non-relapse groups. Horizontal axis indicated samples which were arranged according to NPM1 expression. Vertical axis indicated genes which were arranged according to their absolute r-value. Genes with highest absolute r value were at the top and those with lowest absolute r value were at the bottom. The level of NPM1 was shown at the bottom as reference.

FIG. 59 shows the workflow for the gene expression data analysis process for the staging of lung adenocarcinoma.

FIG. 60 shows the heatmap of Lung adenocarcinoma-Stage I.

FIG. 61 shows the microscopic view of Lung adenocarcinoma-Stage I.

FIG. 62 shows the heatmap of Lung adenocarcinoma-Stage II.

FIGS. 63a and 63b show the microscopic view of Lung adenocarcinoma-Stage II.

FIGS. 64a, 64b and 64c show the heatmap of Lung adenocarcinoma-Stage III, IV.

FIG. 65 shows the microscopic view of Lung adenocarcinoma-Stage III, IV.

FIGS. 66a and 66b show the interactions of functional gene modules of all stages linking to carcinogenesis. NPM1 co-expressed genes are inter-related based on four subgroups of GO biological pathways, which are connected with possible hallmarks of cancer. Common genes in between the pathways are linked with arrows. Genes in different stages under Humoral immune response are shown. With elements obtained from Weinberg, Hanahan, 2011, The Hallmarks of Cancer, diagram by Weinberg & Hanahan, Cell, accessed January 2023, <https://www.cell.com/fulltext/S0092-8674% 2811%2900127-9>.

FIG. 67 shows the study workflow for the gene expression data analysis process for small cell lung cancer (SCLC).

FIG. 68 shows the heatmap of the Small cell lung cancer—MAPK signaling Pathways.

FIG. 69 shows the microscopic view of the Small cell lung cancer—MAPK signaling Pathways.

FIG. 70 shows the microscopic view of the Small cell lung cancer—PI3K/AKT.

FIG. 71 shows the heatmap of the Small cell lung cancer—PI3K/AKT.

FIG. 72 shows the heatmap of the Small cell lung cancer-platinum drug resistance pathway.

FIG. 73 shows the microscopic view of the Small cell lung cancer-platinum drug resistance pathway.

FIG. 74 shows the macroscopic view of the small cell lung cancer-platinum drug resistance pathway.

FIG. 75 shows the Macroscopic view of the Hepatocellular Carcinoma-Interleukin-1 pathway

FIGS. 76a and 76b show the heatmap of the Liver Cancer—Interleukin-1 pathway

FIG. 77 shows the microscopic view of the Liver Cancer—Interleukin-1 pathway.

FIG. 78 shows the macroscopic view of the Liver Cancer—Spliceosome gene Regulation.

FIG. 79 shows the heatmap of the Liver Cancer—Spliceosome gene regulation.

FIG. 80 shows the microscopic view of the Liver Cancer—Spliceosome gene regulation. Map of the gene-gene co-expression module of spliceosome genes and peretinoin drug mechanism. The map links pathways related to peretinoin, spliceosome and inflammation to the characteristics of HCC.

FIG. 81 shows the heatmap of the NFκB signaling network in HBV-associated HCC.

FIG. 82 shows the heatmap of the Prostate Cancer in Metastasis Stage

FIG. 83 shows the microscopic view of the Prostate Cancer in Metastasis Stage.

FIG. 84 shows the macroscopic view of the NFκB signaling network in HBV-associated HCC.

FIG. 85 shows the microscopic view of the NFκB signaling network in HBV-associated HCC.

### DETAILED DESCRIPTION OF THE INVENTION

The present invention a big data analytic platform analyzing NPM1-associated gene expression side-by-side with whole-genome co-expressional changes and the transcriptome-wide gene co-expression network of diseases, and identifying diseases-specific interruption of gene co-expressions. Particularly, this platform not only can be used for the development of genetic markers for diagnosis and therapeutic targets, and the investigation of diseases like viral infections, autoimmune diseases, Alzheimer's disease pathology but also in drug resistance.

The present invention also provides a method to perform Differential Gene Expression Analysis to understand pathway disruption, and Whole Genome Co-expression Analysis to identify disruption in hub genes and NPM1 co-expression networks. All gene sets derived are subjected to functional enrichment.

This invention provides a method for identifying a genetic trait of cells in a state of interest. In one embodiment, said method comprises the steps of: a) Obtaining a first gene expression data from cells in said state of interest; b) Obtaining a second gene expression data from cells in a reference state; c) Conducting one or both of the following steps: 1) Identifying a first set of target genes, wherein each gene in said first set of target genes is strongly co-expressed with another gene in said first set of target genes in said state of interest as compared to said reference state by: i) Conducting a first co-expression analysis on said first gene expression data to arrive at a first co-expression data; ii) Conducting a second co-expression analysis on said second gene expression data to arrive at a second co-expression data; iii) Comparing said first and second co-expression data to identify said first set of target genes; 2) Identifying a second set of target genes, wherein each target gene in said second set of target genes are differentially expressed genes with high connectivity in said state of interest as compared to said reference state by: i) Conducting differential expression analysis on said first gene expression data to identify a set of differentially expressed genes in said state of interest with respect to said reference state; ii) Identify said second set of target genes with high connectivity among said set of differentially expressed genes; d) Identifying a third set of target genes, wherein each target gene in said third set of target genes is strongly co-expressed with NPM1 in said state of interest as compared to said reference state; e) Conducting functional enrichment or pathway enrichment on said target genes obtained from steps (c) to (d); f) Identifying signaling pathways associated with said target genes; and g) Comparing said signaling pathways against a database to identify said genetic trait.

In one embodiment, said state of interest is selected from the group consisting of breast cancer, ovarian cancer, lung cancer, colorectal cancer, small cell lung cancer, liver cancer and prostate cancer.

In one embodiment, said reference state is a healthy state or a state different from said state of interest.

In one embodiment, said genetic trait is selected from the group consisting of cancer reoccurrence, cancer chemoresistance, cancer staging, drug sensitivity, platinum drug resistance, cancer diagnosis, and metastatic cancer staging.

In one embodiment, said state of interest is liver cancer and said genetic trait is liver cancer development from HBV infection.

In one embodiment, said first or second co-expression analysis is selected from one or more of whole genome co-expression analysis, gene co-expression network analysis and weighted gene co-expression network analysis.

In one embodiment, said first gene expression data or said second gene expression data is: a) obtained using Next Generation Sequencing, Openarray technology, qPCR or Microarray technology; or b) retrieved from a data repository.

In one embodiment, said step (d) further comprises identifying one or more sets of target genes, wherein each target gene in said one or more sets of target genes is strongly co-expressed with a gene of interest in said state of interest as compared to said reference state.

In one embodiment, said gene of interest is selected from the group consisting of ERBB2, BRCA1, BRCA2, BARD1, BRIP1, PALB2, RAD51, RAD54L, XRCC3, ERBB2, ESR1, PGR, GATA3, PIK3CA, TP53, PPM1D, RB1CC1, HMMR, NQO2, SLC22A18, PTEN, EGFR, KIT, NOTCH1, NOTCH4, FZD7, LRP6, FGFR1, and CCND1 when said state of interest is breast cancer.

In one embodiment, said gene of interest is selected from the group consisting of BRCA1, BRCA2, MSH2, MLH1, ERBB2, KRAS, AKT2, PIK3CA, MYC, TP53, CTNNB1, PRKN, OPCML, AKT1 and CDH1 when said state of interest is ovarian cancer.

In one embodiment, said gene of interest is selected from the group consisting of ERBB1, TGFA, AREG, EREG, MLH1, MLH3, MSH2, MSH6, TGFBR2, APC, MSH3, POLD1, POLE, DCC, KRAS, GALNT12, SMAD7, SMAD4, SMAD2, BAX, AXIN2, BRAF, CCND1, CHEK2, CTNNB1, FLCN, PIK3CA, TP53, BUB1, BUB1B, AURKA, SERP2, EFEMP2, FBN1, SPARC, and LINC0219 when said state of interest is colorectal cancer.

In one embodiment, said gene of interest is selected from the group consisting of ERBB1, MYC, BCL2, FHIT, TP53, RB1, PTEN, PPP2R1B, EML4-ALK, CD74-ROS1, SLC34A2-ROS1, KIF5B-RET, RARB, RASSF1, KRAS, FHIT, CDKN2A, TP53, MET, BRAF, PIK3CA, IRF1, and PPP2R1B when said state of interest is lung cancer.

In one embodiment, said gene of interest is selected from the group consisting of BCR-ABL, MLL-AF4, E2A-PBX1, TEL-AML1, c-MYC, CRLF2, PAX5, NOTCH1, TAL1, TAL2, LYL1, MLL-ENL, HOX11, MYC, LMO2, HOX11L2, PICALM-MLLT10, PML-RARalpha, AML1-ETO, PLZF-RARalpha, FLT3, KIT, NRAS, KRAS, AML1, CEBPA, CBFB, CHIC2, DNMT3A, ETV6, GATA2, JAK2, LPP, MLLT10, NPM1, NUP214, PICALM, SH3GL1, TERT, BCR-ABL, MECOM, RUNX1, CDKN2A, TP53, RB1, Bcl-2, p53, ATM, Fas, Bcl-6, CyclinD1, p16/INK4A, Fas, KIT, FIPIL1-PDGFRA, BCR-PDGFRA, CBL, TET2, ASXL1, SRSF2, NRAS, KRAS, CBL, RUNX1, SF3B1, ZRSR2, U2AF1, DNMT3A, EZH2, TP53, NPM1, JAK2, FLT3, SETBP1, CSF3R, ETNK1, CEBPA, IDH2, PTPN11, ARHGAP26, NF1, PML-RARA, PLZF-RARA, NUMA1-RARA, CD19, CD22, CD79, CD2, CD3, CD5, and CD8 when said state of interest is leukemia.

In one embodiment, said gene of interest is selected from the group consisting of TGFA, IGF2, IGF1R, TERT, FZD7, HGF, MET, MYC, RB1, CDKN2A, TGFBR2, TP53, PTEN, CTNNB1, AXIN1, KEAP1, NFE2L2, PIK3CA, ARID1A, ARID2, CASP8, and IGF2R when said state of interest is liver cancer.

In one embodiment, said gene of interest is selected from the group consisting of AR, CDKN1B, NKX3.1, PTEN, GSTP1, TMPRSS2-ERG, TMPRSS2-ETV1, TMPRSS2-ETV4, TMPRSS2-ETV5, SLC45A3-ETV1, SLC45A3-ELK4, DDX5-ETV4, MAD1L1, KLF6, MXI1, ZFHX3, BRCA2, BRCA1, ATM, CHEK2, PALB2, MSH2, and MSH6 when said state of interest is prostate cancer.

In one embodiment, connectivity of said second set of target genes with high connectivity is evaluated by one or more methods selected from the group consisting of STRING, Reactome, KEGG, PathCards, Geneck, Cytoscape-ClueGO.

In one embodiment, said database is a library of predetermined relationship between said signaling pathways and said genetic trait.

In one embodiment, significance of co-expression of said first set of target genes is determined using one or more of the methods selected from the group consisting of Pearson correlation coefficient, Pearson product-moment correlation coefficient, cosine-angle uncentered correlation, cosine correlation, (non parametric) Kendall rank correlation and Spearman correlation, coefficient of determination (the R-squared measure of goodness of fit), Lack-of-fit sum of squares, Reduced chi-square, Regression validation, Mallows's Cp criterion, Bayesian information criterion, Kolmogorov-Smirnov test, Cramér-von Mises criterion, Anderson-Darling test, Shapiro-Wilk test, Chi-squared test, Akaike information criterion, Hosmer-Lemeshow test, Kuiper's test, Kernelized Stein discrepancy, Zhang's ZK, ZC and ZA tests, Moran test, Density Based Empirical Likelihood Ratio tests and Two-sample Kolmogorov-Smirnov test.

In one embodiment, said step (f) further comprises analyzing transcription factors associated with said genes.

This invention also provides a computer-implemented method for identifying a genetic trait of cells in a state of interest. In one embodiment, said computer-implemented method comprises the steps of: a) Obtaining a first gene expression data from cells in said state of interest; b) Obtaining a second gene expression data from cells in a reference state; c) Conducting one or both of the following steps: 1) Identifying a first set of target genes, wherein each gene in said first set of target genes is strongly co-expressed with another gene in said first set of target genes in said state of interest as compared to said reference state by: i) Conducting a first co-expression analysis on said first gene expression data to arrive at a first co-expression data; ii) Conducting a second co-expression analysis on said second gene expression data to arrive at a second co-expression data; iii) Comparing said first and second co-expression data to identify said first set of target genes; 2) Identifying a second set of target genes, wherein each target gene in said second set of target genes are differentially expressed genes with high connectivity in said state of interest as compared to said reference state by: i) Conducting differential expression analysis on said first gene expression data to identify a set of differentially expressed genes in said state of interest with respect to said reference state; ii) Identify said second set of target genes with high connectivity among said set of differentially expressed genes; d) Identifying a third set of target genes, wherein each target gene in said third set of target genes is strongly co-expressed with NPM1 in said state of interest as compared to said reference state; e) Conducting functional enrichment or pathway enrichment on said target genes obtained from steps (c) to (d); f) Identifying signaling pathways associated with said target genes; and g) Comparing said signaling pathways against a database to identify said genetic trait.

In one embodiment, said state of interest is selected from the group consisting of breast cancer, ovarian cancer, lung cancer, colorectal cancer, small cell lung cancer, liver cancer and prostate cancer.

In one embodiment, said reference state is a healthy state or a state different from said state of interest.

In one embodiment, said genetic trait is selected from the group consisting of cancer reoccurrence, cancer chemoresistance, cancer staging, drug sensitivity, platinum drug resistance, cancer diagnosis, and metastatic cancer staging.

In one embodiment, said state of interest is liver cancer and said genetic trait is liver cancer development from HBV infection.

In one embodiment, said first or second co-expression analysis is selected from one or more of whole genome co-expression analysis, gene co-expression network analysis and weighted gene co-expression network analysis.

In one embodiment, said first gene expression data or said second gene expression data is: a) obtained using Next Generation Sequencing, Openarray technology, qPCR or Microarray technology; or b) retrieved from a data repository.

In one embodiment, said step (d) further comprises identifying one or more sets of target genes, wherein each target gene in said one or more sets of target genes is strongly co-expressed with a gene of interest in said state of interest as compared to said reference state.

In one embodiment, said gene of interest is selected from the group consisting of ERBB2, BRCA1, BRCA2, BARD1, BRIP1, PALB2, RAD51, RAD54L, XRCC3, ERBB2, ESR1, PGR, GATA3, PIK3CA, TP53, PPM1D, RB1CC1, HMMR, NQO2, SLC22A18, PTEN, EGFR, KIT, NOTCH1, NOTCH4, FZD7, LRP6, FGFR1, and CCND1 when said state of interest is breast cancer.

In one embodiment, said gene of interest is selected from the group consisting of BRCA1, BRCA2, MSH2, MLH1, ERBB2, KRAS, AKT2, PIK3CA, MYC, TP53, CTNNB1, PRKN, OPCML, AKT1 and CDH1 when said state of interest is ovarian cancer.

In one embodiment, said gene of interest is selected from the group consisting of ERBB1, TGFA, AREG,EREG, MLH1, MLH3, MSH2, MSH6, TGFBR2, APC, MSH3, POLD1, POLE, DCC, KRAS, GALNT12, SMAD7, SMAD4, SMAD2, BAX, AXIN2, BRAF, CCND1, CHEK2, CTNNB1, FLCN, PIK3CA, TP53, BUB1, BUB1B, AURKA, SERP2, EFEMP2, FBN1, SPARC, and LINC0219 when said state of interest is colorectal cancer.

In one embodiment, said gene of interest is selected from the group consisting of ERBB1, MYC, BCL2, FHIT, TP53, RB1, PTEN, PPP2R1B, EML4-ALK, CD74-ROS1, SLC34A2-ROS1, KIF5B-RET, RARB, RASSF1, KRAS, FHIT, CDKN2A, TP53, MET, BRAF, PIK3CA, IRF1, and PPP2R1B when said state of interest is lung cancer.

In one embodiment, said gene of interest is selected from the group consisting of BCR-ABL, MLL-AF4, E2A-PBX1, TEL-AML1, c-MYC, CRLF2, PAX5, NOTCH1, TAL1, TAL2, LYL1, MLL-ENL, HOX11, MYC, LMO2, HOX11L2, PICALM-MLLT10, PML-RARalpha, AML1-ETO, PLZF-RARalpha, FLT3, KIT, NRAS, KRAS, AML1, CEBPA, CBFB, CHIC2, DNMT3A, ETV6, GATA2, JAK2, LPP, MLLT10, NPM1, NUP214, PICALM, SH3GL1, TERT, BCR-ABL, MECOM, RUNX1, CDKN2A, TP53, RB1, Bcl-2, p53, ATM, Fas, Bcl-6, CyclinD1, p16/INK4A, Fas, KIT, FIP1L1-PDGFRA, BCR-PDGFRA, CBL, TET2, ASXL1, SRSF2, NRAS, KRAS, CBL, RUNX1, SF3B1, ZRSR2, U2AF1, DNMT3A, EZH2, TP53, NPM1, JAK2, FLT3, SETBP1, CSF3R, ETNK1, CEBPA, IDH2, PTPN11, ARHGAP26, NF1, PML-RARA, PLZF-RARA, NUMA1-RARA, CD19, CD22, CD79, CD2, CD3, CD5, and CD8 when said state of interest is leukemia.

In one embodiment, said gene of interest is selected from the group consisting of TGFA, IGF2, IGF1R, TERT, FZD7, HGF, MET, MYC, RB1, CDKN2A, TGFBR2, TP53, PTEN, CTNNB1, AXIN1, KEAP1, NFE2L2, PIK3CA, ARID1A, ARID2, CASP8, and IGF2R when said state of interest is liver cancer.

In one embodiment, said gene of interest is selected from the group consisting of AR, CDKN1B, NKX3.1, PTEN, GSTP1, TMPRSS2-ERG, TMPRSS2-ETV1, TMPRSS2-ETV4, TMPRSS2-ETV5, SLC45A3-ETV1, SLC45A3-ELK4, DDX5-ETV4, MAD1L1, KLF6, MXI1, ZFHX3, BRCA2, BRCA1, ATM, CHEK2, PALB2, MSH2, and MSH6 when said state of interest is prostate cancer.

In one embodiment, connectivity of said second set of target genes with high connectivity is evaluated by one or more methods selected from the group consisting of STRING, Reactome, KEGG, PathCards, Geneck, Cytoscape-ClueGO.

In one embodiment, said database is a library of predetermined relationship between said signaling pathways and said genetic trait.

In one embodiment, significance of co-expression of said first set of target genes is determined using one or more of the methods selected from the group consisting of Pearson correlation coefficient, Pearson product-moment correlation coefficient, cosine-angle uncentered correlation, cosine correlation, (non parametric) Kendall rank correlation and Spearman correlation, coefficient of determination (the R-squared measure of goodness of fit), Lack-of-fit sum of squares, Reduced chi-square, Regression validation, Mallows's Cp criterion, Bayesian information criterion, Kolmogorov-Smirnov test, Cramér-von Mises criterion, Anderson-Darling test, Shapiro-Wilk test, Chi-squared test, Akaike information criterion, Hosmer-Lemeshow test, Kuiper's test, Kernelized Stein discrepancy, Zhang's ZK, ZC and ZA tests, Moran test, Density Based Empirical Likelihood Ratio tests and Two-sample Kolmogorov-Smirnov test.

In one embodiment, said step (f) further comprises analyzing transcription factors associated with said genes.

This invention also provides a non-transitory computer-readable medium having stored thereon program instructions that, upon execution by a computing device, cause the computing device to perform operations for identifying a genetic trait of cells in a state of interest. In one embodiment, said operations comprises the steps of: a) Obtaining a first gene expression data from cells in said state of interest; b) Obtaining a second gene expression data from cells in a reference state; c) Conducting one or both of the following steps: 1) Identifying a first set of target genes, wherein each gene in said first set of target genes is strongly co-expressed with another gene in said first set of target genes in said state of interest as compared to said reference state by: i) Conducting a first co-expression analysis on said first gene expression data to arrive at a first co-expression data; ii) Conducting a second co-expression analysis on said second gene expression data to arrive at a second co-expression data; iii) Comparing said first and second co-expression data to identify said first set of target genes; 2) Identifying a second set of target genes, wherein each target gene in said second set of target genes are differentially expressed genes with high connectivity in said state of interest as compared to said reference state by: i) Conducting differential expression analysis on said first gene expression data to identify a set of differentially expressed genes in said state of interest with respect to said reference state; ii) Identify said second set of target genes with high connectivity among said set of differentially expressed genes; d) Identifying a third set of target genes, wherein each target gene in said third set of target genes is strongly co-expressed with NPM1 in said state of interest as compared to said reference state; e) Conducting functional enrichment or pathway enrichment on said target genes obtained from steps (c) to (d); f) Identifying signaling pathways associated with said target genes; and g) Comparing said signaling pathways against a database to identify said genetic trait.

In one embodiment, said state of interest is selected from the group consisting of breast cancer, ovarian cancer, lung cancer, colorectal cancer, small cell lung cancer, liver cancer and prostate cancer.

In one embodiment, said reference state is a healthy state or a state different from said state of interest.

In one embodiment, said genetic trait is selected from the group consisting of cancer reoccurrence, cancer chemoresistance, cancer staging, drug sensitivity, platinum drug resistance, cancer diagnosis, and metastatic cancer staging.

In one embodiment, said state of interest is liver cancer and said genetic trait is liver cancer development from HBV infection.

In one embodiment, said first or second co-expression analysis is selected from one or more of whole genome co-expression analysis, gene co-expression network analysis and weighted gene co-expression network analysis.

In one embodiment, said first gene expression data or said second gene expression data is: a) obtained using Next Generation Sequencing, Openarray technology, qPCR or Microarray technology; or b) retrieved from a data repository.

In one embodiment, said step (d) further comprises identifying one or more sets of target genes, wherein each target gene in said one or more sets of target genes is strongly co-expressed with a gene of interest in said state of interest as compared to said reference state.

In one embodiment, said gene of interest is selected from the group consisting of ERBB2, BRCA1, BRCA2, BARD1, BRIP1, PALB2, RAD51, RAD54L, XRCC3, ERBB2, ESR1, PGR, GATA3, PIK3CA, TP53, PPM1D, RB1CC1, HMMR, NQO2, SLC22A18, PTEN, EGFR, KIT, NOTCH1, NOTCH4, FZD7, LRP6, FGFR1, and CCND1 when said state of interest is breast cancer.

In one embodiment, said gene of interest is selected from the group consisting of BRCA1, BRCA2, MSH2, MLH1, ERBB2, KRAS, AKT2, PIK3CA, MYC, TP53, CTNNB1, PRKN, OPCML, AKT1 and CDH1 when said state of interest is ovarian cancer.

In one embodiment, said gene of interest is selected from the group consisting of ERBB1, TGFA, AREG, EREG, MLH1, MLH3, MSH2, MSH6, TGFBR2, APC, MSH3, POLD1, POLE, DCC, KRAS, GALNT12, SMAD7, SMAD4, SMAD2, BAX, AXIN2, BRAF, CCND1, CHEK2, CTNNB1, FLCN, PIK3CA, TP53, BUB1, BUB1B, AURKA, SERP2, EFEMP2, FBN1, SPARC, and LINC0219 when said state of interest is colorectal cancer.

In one embodiment, said gene of interest is selected from the group consisting of ERBB1, MYC, BCL2, FHIT, TP53, RB1, PTEN, PPP2R1B, EML4-ALK, CD74-ROS1, SLC34A2-ROS1, KIF5B-RET, RARB, RASSF1, KRAS, FHIT, CDKN2A, TP53, MET, BRAF, PIK3CA, IRF1, and PPP2R1B when said state of interest is lung cancer.

In one embodiment, said gene of interest is selected from the group consisting of BCR-ABL, MLL-AF4, E2A-PBX1, TEL-AML1, c-MYC, CRLF2, PAX5, NOTCH1, TAL1, TAL2, LYL1, MLL-ENL, HOX11, MYC, LMO2, HOX11L2, PICALM-MLLT10, PML-RARalpha, AML1-ETO, PLZF-RARalpha, FLT3, KIT, NRAS, KRAS, AML1, CEBPA, CBFB, CHIC2, DNMT3A, ETV6, GATA2, JAK2, LPP, MLLT10, NPM1, NUP214, PICALM, SH3GL1, TERT, BCR-ABL, MECOM, RUNX1, CDKN2A, TP53, RB1, Bcl-2, p53, ATM, Fas, Bcl-6, CyclinD1, p16/INK4A, Fas, KIT, FIP1L1-PDGFRA, BCR-PDGFRA, CBL, TET2, ASXL1, SRSF2, NRAS, KRAS, CBL, RUNX1, SF3B1, ZRSR2, U2AF1, DNMT3A, EZH2, TP53, NPM1, JAK2, FLT3, SETBP1, CSF3R, ETNK1, CEBPA, IDH2, PTPN11, ARHGAP26, NF1, PML-RARA, PLZF-RARA, NUMA1-RARA, CD19, CD22, CD79, CD2, CD3, CD5, and CD8 when said state of interest is leukemia.

In one embodiment, said gene of interest is selected from the group consisting of TGFA, IGF2, IGF1R, TERT, FZD7, HGF, MET, MYC, RB1, CDKN2A, TGFBR2, TP53, PTEN, CTNNB1, AXIN1, KEAP1, NFE2L2, PIK3CA, ARID1A, ARID2, CASP8, and IGF2R when said state of interest is liver cancer.

In one embodiment, said gene of interest is selected from the group consisting of AR, CDKN1B, NKX3.1, PTEN, GSTP1, TMPRSS2-ERG, TMPRSS2-ETV1, TMPRSS2-ETV4, TMPRSS2-ETV5, SLC45A3-ETV1, SLC45A3-ELK4, DDX5-ETV4, MAD1L1, KLF6, MXI1, ZFHX3, BRCA2, BRCA1, ATM, CHEK2, PALB2, MSH2, and MSH6 when said state of interest is prostate cancer.

In one embodiment, connectivity of said second set of target genes with high connectivity is evaluated by one or more methods selected from the group consisting of STRING, Reactome, KEGG, PathCards, Geneck, Cytoscape-ClueGO.

In one embodiment, said database is a library of predetermined relationship between said signaling pathways and said genetic trait.

In one embodiment, significance of co-expression of said first set of target genes is determined using one or more of the methods selected from the group consisting of Pearson correlation coefficient, Pearson product-moment correlation coefficient, cosine-angle uncentered correlation, cosine correlation, (non parametric) Kendall rank correlation and Spearman correlation, coefficient of determination (the R-squared measure of goodness of fit), Lack-of-fit sum of squares, Reduced chi-square, Regression validation, Mallows's Cp criterion, Bayesian information criterion, Kolmogorov-Smirnov test, Cramer-von Mises criterion, Anderson-Darling test, Shapiro-Wilk test, Chi-squared test, Akaike information criterion, Hosmer-Lemeshow test, Kuiper's test, Kernelized Stein discrepancy, Zhang's ZK, ZC and ZA tests, Moran test, Density Based Empirical Likelihood Ratio tests and Two-sample Kolmogorov-Smirnov test.

In one embodiment, said step (f) further comprises analyzing transcription factors associated with said genes.

This invention further provides a computing device comprising: 1) a processor; 2) memory; and 3) program instructions, stored in the memory, that upon execution by the processor cause the computing device to perform operations for identifying a genetic trait of cells in a state of interest. In one embodiment, said operations comprises the steps of: a) Obtaining a first gene expression data from cells in said state of interest; b) Obtaining a second gene expression data from cells in a reference state; c) Conducting one or both of the following steps: 1) Identifying a first set of target genes, wherein each gene in said first set of target genes is strongly co-expressed with another gene in said first set of target genes in said state of interest as compared to said reference state by: i) Conducting a first co-expression analysis on said first gene expression data to arrive at a first co-expression data; ii) Conducting a second co-expression analysis on said second gene expression data to arrive at a second co-expression data; iii) Comparing said first and second co-expression data to identify said first set of target genes; 2) Identifying a second set of target genes, wherein each target gene in said second set of target genes are differentially expressed genes with high connectivity in said state of interest as compared to said reference state by: i) Conducting differential expression analysis on said first gene expression data to identify a set of differentially expressed genes in said state of interest with respect to said reference state; ii) Identify said second set of target genes with high connectivity among said set of differentially expressed genes; d) Identifying a third set of target genes, wherein each target gene in said third set of target genes is strongly co-expressed with NPM1 in said state of interest as compared to said reference state; e) Conducting functional enrichment or pathway enrichment on said target genes obtained from steps (c) to (d); f) Identifying signaling pathways associated with said target genes; and g) Comparing said signaling pathways against a database to identify said genetic trait.

In one embodiment, said state of interest is selected from the group consisting of breast cancer, ovarian cancer, lung cancer, colorectal cancer, small cell lung cancer, liver cancer and prostate cancer.

In one embodiment, said reference state is a healthy state or a state different from said state of interest.

In one embodiment, said genetic trait is selected from the group consisting of cancer reoccurrence, cancer chemoresistance, cancer staging, drug sensitivity, platinum drug resistance, cancer diagnosis, and metastatic cancer staging.

In one embodiment, said state of interest is liver cancer and said genetic trait is liver cancer development from HBV infection.

In one embodiment, said first or second co-expression analysis is selected from one or more of whole genome co-expression analysis, gene co-expression network analysis and weighted gene co-expression network analysis.

In one embodiment, said first gene expression data or said second gene expression data is: a) obtained using Next Generation Sequencing, Openarray technology, qPCR or Microarray technology; or b) retrieved from a data repository.

In one embodiment, said step (d) further comprises identifying one or more sets of target genes, wherein each target gene in said one or more sets of target genes is strongly co-expressed with a gene of interest in said state of interest as compared to said reference state.

In one embodiment, said gene of interest is selected from the group consisting of ERBB2, BRCA1, BRCA2, BARD1, BRIP1, PALB2, RAD51, RAD54L, XRCC3, ERBB2, ESR1, PGR, GATA3, PIK3CA, TP53, PPM1D, RB1CC1, HMMR, NQO2, SLC22A18, PTEN, EGFR, KIT, NOTCH1, NOTCH4, FZD7, LRP6, FGFR1, and CCND1 when said state of interest is breast cancer.

In one embodiment, said gene of interest is selected from the group consisting of BRCA1, BRCA2, MSH2, MLH1, ERBB2, KRAS, AKT2, PIK3CA, MYC, TP53, CTNNB1, PRKN, OPCML, AKT1 and CDH1 when said state of interest is ovarian cancer.

In one embodiment, said gene of interest is selected from the group consisting of ERBB1, TGFA, AREG,EREG, MLH1, MLH3, MSH2, MSH6, TGFBR2, APC, MSH3, POLD1, POLE, DCC, KRAS, GALNT12, SMAD7, SMAD4, SMAD2, BAX, AXIN2, BRAF, CCND1, CHEK2, CTNNB1, FLCN, PIK3CA, TP53, BUB1, BUB1B, AURKA, SERP2, EFEMP2, FBN1, SPARC, and LINC0219 when said state of interest is colorectal cancer.

In one embodiment, said gene of interest is selected from the group consisting of ERBB1, MYC, BCL2, FHIT, TP53, RB1, PTEN, PPP2R1B, EML4-ALK, CD74-ROS1, SLC34A2-ROS1, KIF5B-RET, RARB, RASSF1, KRAS, FHIT, CDKN2A, TP53, MET, BRAF, PIK3CA, IRF1, and PPP2R1B when said state of interest is lung cancer.

In one embodiment, said gene of interest is selected from the group consisting of BCR-ABL, MLL-AF4, E2A-PBX1, TEL-AML1, c-MYC, CRLF2, PAX5, NOTCH1, TAL1, TAL2, LYL1, MLL-ENL, HOX11, MYC, LMO2, HOX11L2, PICALM-MLLT10, PML-RARalpha, AML1-ETO, PLZF-RARalpha, FLT3, KIT, NRAS, KRAS, AML1, CEBPA, CBFB, CHIC2, DNMT3A, ETV6, GATA2, JAK2, LPP, MLLT10, NPM1, NUP214, PICALM, SH3GL1, TERT, BCR-ABL, MECOM, RUNX1, CDKN2A, TP53, RB1, Bcl-2, p53, ATM, Fas, Bcl-6, CyclinD1, p16/INK4A, Fas, KIT, FIPIL1-PDGFRA, BCR-PDGFRA, CBL, TET2, ASXL1, SRSF2, NRAS, KRAS, CBL, RUNX1, SF3B1, ZRSR2, U2AF1, DNMT3A, EZH2, TP53, NPM1, JAK2, FLT3, SETBP1, CSF3R, ETNK1, CEBPA, IDH2, PTPN11, ARHGAP26, NF1, PML-RARA, PLZF-RARA, NUMA1-RARA, CD19, CD22, CD79, CD2, CD3, CD5, and CD8 when said state of interest is leukemia.

In one embodiment, said gene of interest is selected from the group consisting of TGFA, IGF2, IGF1R, TERT, FZD7, HGF, MET, MYC, RB1, CDKN2A, TGFBR2, TP53, PTEN, CTNNB1, AXIN1, KEAP1, NFE2L2, PIK3CA, ARID1A, ARID2, CASP8, and IGF2R when said state of interest is liver cancer.

In one embodiment, said gene of interest is selected from the group consisting of AR, CDKN1B, NKX3.1, PTEN, GSTP1, TMPRSS2-ERG, TMPRSS2-ETV1, TMPRSS2-ETV4, TMPRSS2-ETV5, SLC45A3-ETV1, SLC45A3-ELK4, DDX5-ETV4, MAD1L1, KLF6, MXI1, ZFHX3, BRCA2, BRCA1, ATM, CHEK2, PALB2, MSH2, and MSH6 when said state of interest is prostate cancer.

In one embodiment, connectivity of said second set of target genes with high connectivity is evaluated by one or more methods selected from the group consisting of STRING, Reactome, KEGG, PathCards, Geneck, Cytoscape-ClueGO.

In one embodiment, said database is a library of predetermined relationship between said signaling pathways and said genetic trait.

In one embodiment, significance of co-expression of said first set of target genes is determined using one or more of the methods selected from the group consisting of Pearson correlation coefficient, Pearson product-moment correlation coefficient, cosine-angle uncentered correlation, cosine correlation, (non parametric) Kendall rank correlation and Spearman correlation, coefficient of determination (the R-squared measure of goodness of fit), Lack-of-fit sum of squares, Reduced chi-square, Regression validation, Mallows's Cp criterion, Bayesian information criterion, Kolmogorov-Smirnov test, Cramer-von Mises criterion, Anderson-Darling test, Shapiro-Wilk test, Chi-squared test, Akaike information criterion, Hosmer-Lemeshow test, Kuiper's test, Kernelized Stein discrepancy, Zhang's ZK, ZC and ZA tests, Moran test, Density Based Empirical Likelihood Ratio tests and Two-sample Kolmogorov-Smirnov test.

In one embodiment, said step (f) further comprises analyzing transcription factors associated with said genes.

As compared to other platforms for analysing gene co-expression/interaction so as to identify genetic traits, this invention makes use of clinical patient co-expression data of NPM1 and genes that are significantly associated with NPM1 in states of interest. Prior arts, such as Chan et al., 2015, do not include steps for further prediction e.g. patient chemoresistance or other states of interest using NPM1 gene-coexpression data. Pathways involving the NPM1 co-expressed genes are identified using bioinformatics tools. Heatmaps are used for identifying and differentiating between the co-expression pattern in the reference state and the state of interest in order to predict a characteristic, such as cancer recurrence or chemoresistance.

Extensive knowledge of NPM1's role in cancer mechanisms and processes and utilizes data of genes correlated and coexpressed with NPM1 is required to predict a characteristic, such as cancer recurrence. In order to predict a characteristic, such as cancer recurrence, this invention requires consecutively combining global gene co-expression analysis, NPM1 gene co-expression analysis, heatmap construction and pathway enrichment analysis.

The invention will be better understood by reference to the Experimental Details which follow, but those skilled in the art will readily appreciate that the specific experiments described are only for illustrative purpose and are not meant to limit the invention as described herein, which is defined by the claims that follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

### Analysis Workflow

Differential Gene Expression Analysis: Gene expression (RNAs) dataset obtained from the human tissue using methods such as Next Generation Sequencing and Microarray technologies or any other methods known in the art will be analyzed. Key words of inquiry and selection criteria are keyed in. Dataset satisfies all the criteria listed, and the normalized dataset is downloaded for co-expression analysis. Differential gene expression analysis is performed using Welch's t-test (fold change≥1.5, p-value<0.05) with an aim to examine the biological networks of gene interactions. Gene list of interest was submitted to TOPPFUN (Transcriptome, ontology, phenotype proteome, and pharmacome annotations based gene list functional enrichment analysis) software (https://toppgene.cchmc.org/enrichment.jsp) for functional enrichment analysis. The software offers three types of FDR corrections namely, Bonferroni, Benjamini-Hochberg, and Benjamini-Yekutieli. Hub genes are defined as genes with high connectivity. Search Tool for the Retrieval of Interacting Genes/Proteins (STRING) was used for constructing protein-protein interaction network and to evaluate connectivity of differentially expressed genes. Top differentially expressed genes with highest connectivity are selected. Transcription factors from the differentially expressed gene set are identified using two transcription factor databases—Transcriptional Regulatory Relationships Unraveled by Sentence-based Text mining (TRRUST) curated by Yonsei University and TF checkpoint curated by the Norwegian University of Science and Technology. Only transcription factors with a fold change≥2 are considered. Differential Gene Expression Analysis: The Kaplan-Meier estimator and log-rank test are used to construct the survival curves and evaluate their significance (p<0.05). R software (version 4.0.1, www.r-project org) and the survival package are used for graphing.

Whole-genome Co-expression Network Analysis: Cellular processes are a collection of highly regulated signaling events. These signaling pathways require the tight cooperation of an assembly of proteins. The Co-expression analysis explores and understands the intricacy of these networks and how disruption to it provokes disease development. The genome-wide structural co-expression analysis method (NPM1 co-expression network analysis and Target genes co-expression network analysis) previously published by Chan et al., 2015 was used. Pearson correlation coefficient (r) was calculated from all possible gene pairs in each group. Two-sample Kolmogorov-Smirnov test was used to examine whether the two state-specific sets of correlation coefficients significantly differed in overall cumulative distributions. At the maximum deviation between the two curves, a threshold (Rt) was identified and used to classify co-expressed gene pairs into strong and weak co-expressions. This approach hypothesized that gene co-expression patterns from two condition groups (i.e., normal versus disease state) form different distributions. Gene list of interest is submitted to TOPPFUN (Transcriptome, ontology, phenotype, proteome, and pharmacome annotations based gene list functional enrichment analysis) software (https://toppgene.cchmc.org/enrichment.jsp) for functional enrichment analysis. The software offered three types of FDR corrections namely, Bonferroni, Benjamini-Hochberg, and Benjamini-Yekutieli. Processes that satisfied at least two of the three FDR corrections (corrected p-value<0.05) were considered.

Functional and pathway enrichment analysis were processed by Cytoscape. Gene ontology (GO) enrichment analysis was performed for the identified coexpressed genes. The Holm-Bonferroni method was adopted in ClueGO to correct the calculated p-value of identified biological pathways. DAVID (https://david.ncifcrf.gov/summary.jsp), as the most common online tool for functional and pathway enrichment analysis, is adopted to analyze associated co-expressed genes among identified biological pathways. To evaluate the functional interaction among co-expressed genes, a Search Tool for the Retrieval of Interacting Genes (STRING, https://string-db.org/) is implemented to map identified co-expressed genes as in a PPI network. Heatmaps are constructed to illustrate the expression patterns of significantly coexpressed genes by Heatmapper, an online tool for heatmap generation (Heatmapper, http://heatmapper.ca/) or other similar techniques that enables the illustration of the expression patterns such as the heatmapz, package (heatmapz, https://pypi.org/project/heatmapz/), and for the construction of Standard Curve of Co-Expression.

### Example 2

### Reoccurrence of HER2-Positive Breast Cancer

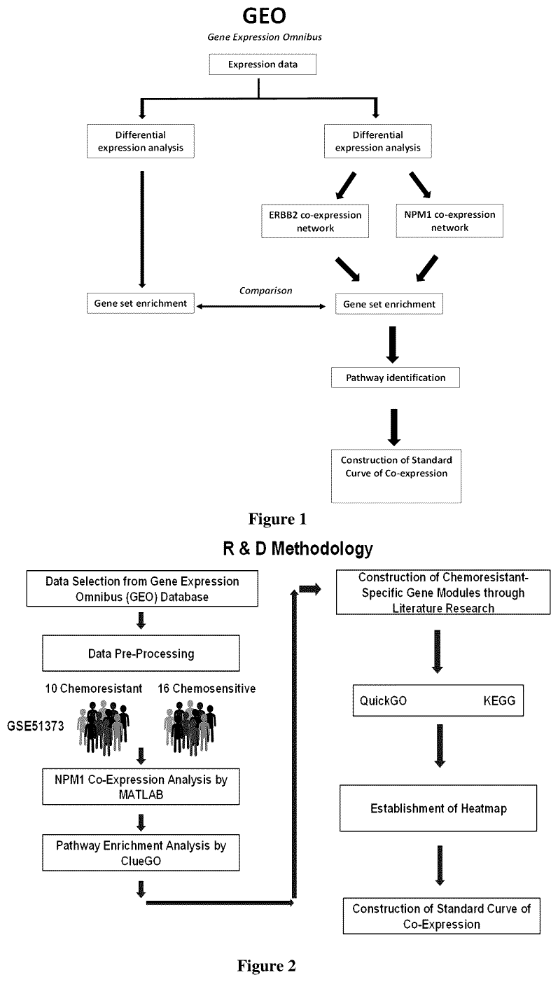

In one embodiment, this invention provides a method (FIG. 1) for diagnosing and predicting the reoccurrence of HER2-positive breast cancer. A sample is first obtained from a subject and gene expression (RNAs) was analyzed with whole-genome co-expressional changes using methods such as Next Generation Sequencing and Microarray technologies or any other methods known in the art. Bioinformatics tools were then used for conducting i) Differential Gene Expression Analysis: Genes (GRB7, PGAP3, MIEN1, ERBB2, ORMDL3, VPS37B, PLCH1, DERL3, SOX11) involved in the immune process are down-regulated; ii) ERBB2 and NPM1 Structural Gene Co-expression Analysis & Functional and Pathway Enrichment: Genes (ERBB2, NPM1, IFNG, STAT1, HLA-DQB2, B2M, FCGR1A, TRIM62, PTAFR, and VCAM1) in the interferon-gamma (IFNG)-mediated signaling involved in immune surveillance and tumor suppression; and iii) NPM1 Structural Gene Co-expression Analysis & Functional and Pathway Enrichment: Analyzing Genes in DNA repair (Table 1.) and Genes in MAPK signaling (Table 2.). Table A shows the conditions used in this Example.

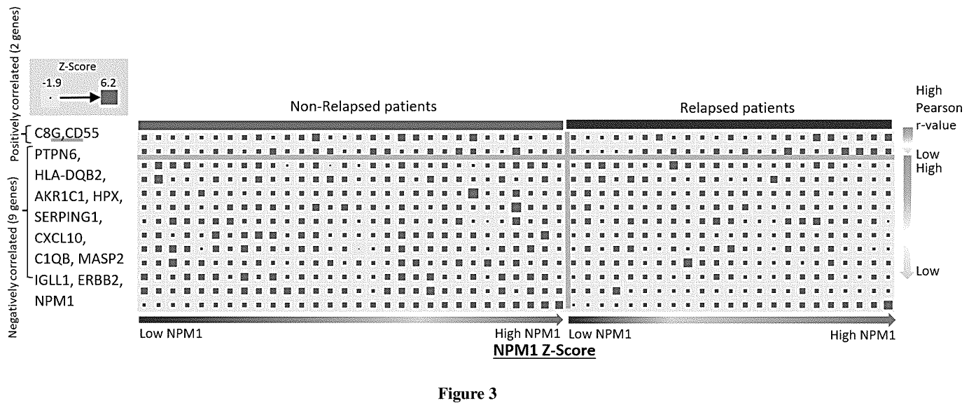

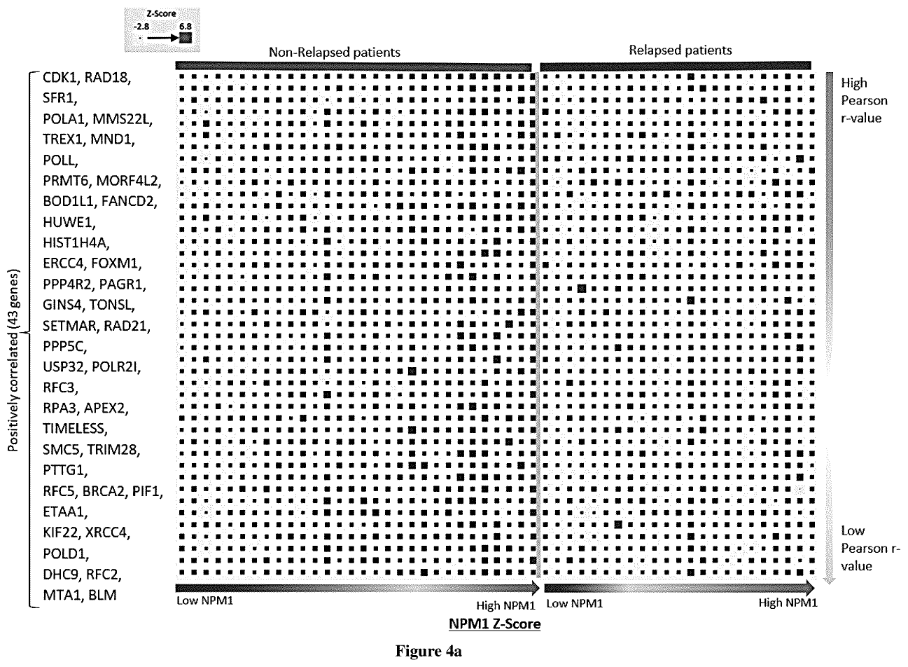

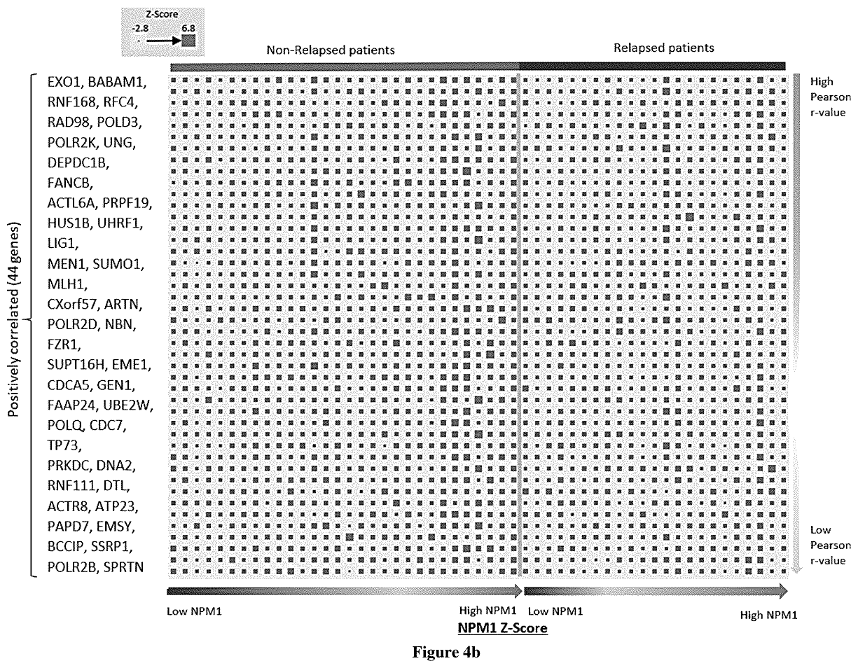

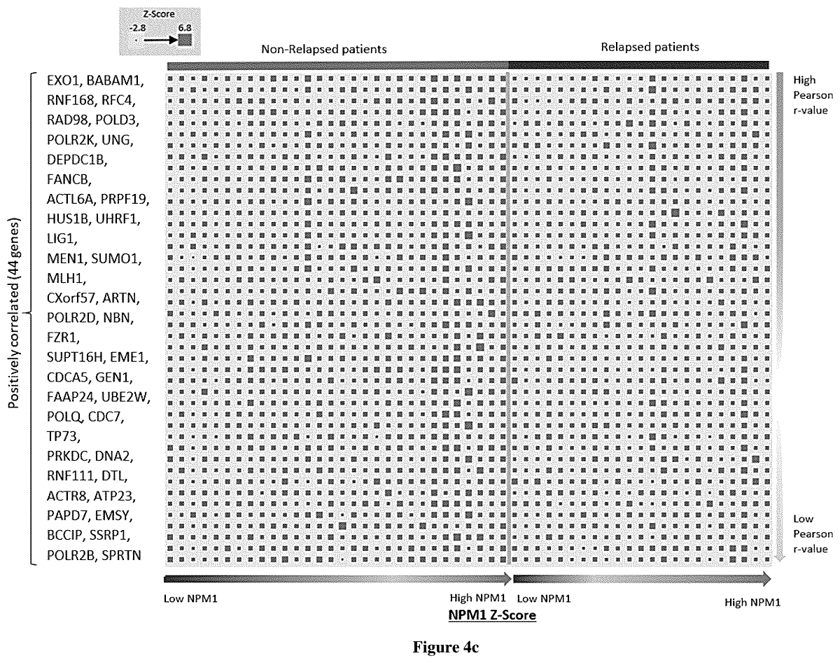

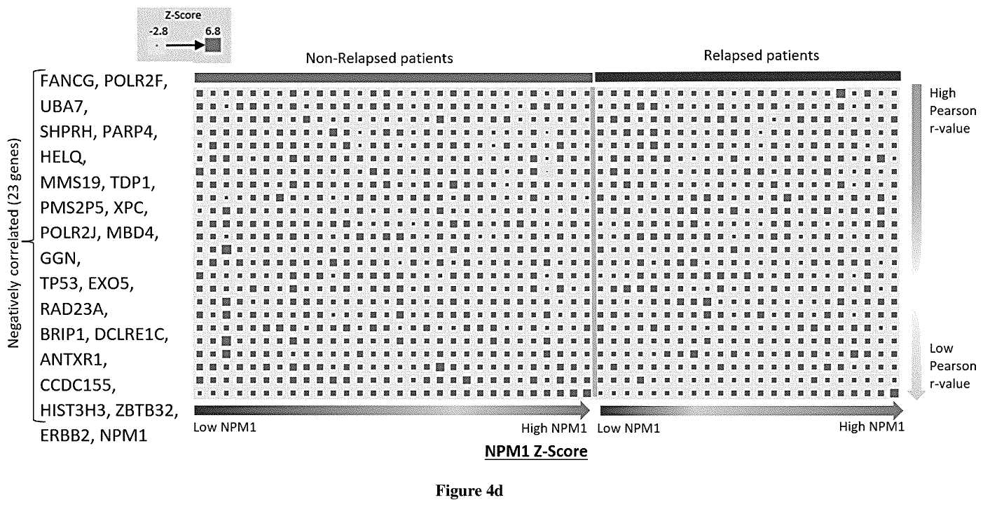

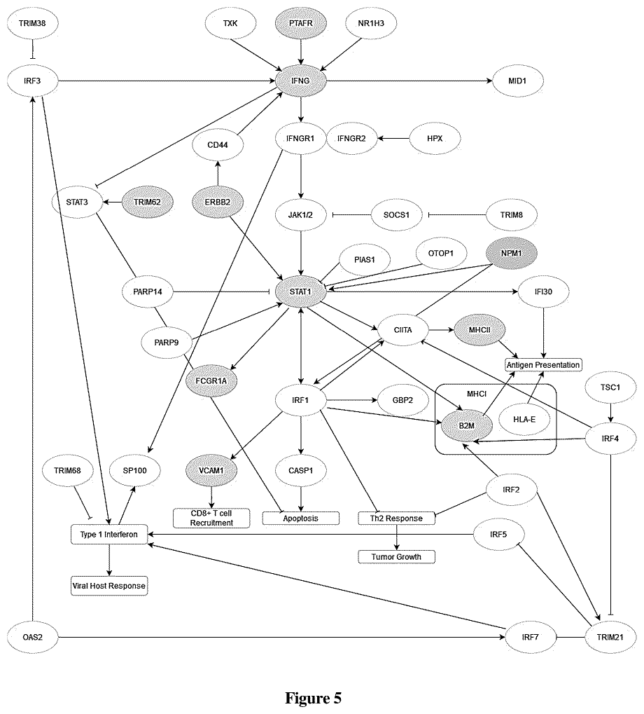

TABLE A
Conditions used in Example 2

| Parameter | | Condition |
|---|---|---|
| Genetic trait | | Cancer recurrence |
| State of interest | | HER2-positive breast cancer |
| | | cells under question |
| Reference state | | HER2-positive breast cancer |
| | | cells known to reoccur |
| Gene of interest | | NPM1-correlated genes appearing |
| | | in FIGS. 3, 4a, 4b, 4c, 4d, 5 |
| Methods | First gene | Cancer patients exhibiting |
| | expression data | recurrence |
| | Second gene | Cancer patients without |
| | expression data | recurrence |
| | First co-expression | NPM1 co-expression analysis |
| | analysis | with MATLAB |
| | Second co-expression | NPM1 co-expression analysis |
| | analysis | with MATLAB |
| | Differential expression | Welch's Test |
| | analysis | |
| | Connectivity | STRING |
| | functional enrichment or | ClueGO, Reactome, KEGG, |
| | pathway enrichment | TOPPFUN |

TABLE 1
Functional enrichment of NPM1 non-relapse specific gene pairs segregated into
positive and negative co-expressions. All Gene Ontology terms are significant
(corrected p-value <0.05, FDR B&H). Terms with corrected p-value
<0.01 are marked with *, and <0.001 are marked with **.

| Positive co-expression | | Negative co-expression | |
|---|---|---|---|
| GO ID | GO Name | GO ID | GO Name |
| GO:0090592 | DNA synthesis involved in | GO:0007267 | Cell-cell signaling** |
| | DNA replication** | | |
| GO:0006271 | DNA strand elongation | GO:0030198 | Extracellular matrix |
| | involved in DNA | | organization** |
| | replication** | | |
| GO:0007093 | Mitotic cell cycle | GO:0006954 | Inflammatory response** |
| | checkpoint** | | |
| GO:0090068 | Positive regulation of cell | GO:0051241 | Negative regulation of |
| | cycle process** | | multicellular organismal |
| | | | process** |
| GO:0006284 | Base-excision repair** | GO:0007186 | G protein-coupled receptor |
| | | | signaling pathway** |
| GO:0051301 | Cell division** | GO:0071495 | Cellular response to |
| | | | endogenous stimulus** |
| GO:0097712 | Vesicle targeting, trans- | GO:0030593 | Neutrophil chemotaxis** |
| | Golgi to periciliary | | |
| | membrane | | |
| | compartment** | | |
| GO:0035082 | Axoneme assembly** | GO:0070374 | Positive regulation of |
| | | | ERK1 and ERK2 |
| | | | cascade** |
| GO:1905349 | Ciliary transition zone | GO:1905606 | Regulation of presynapse |
| | assembly** | | assembly** |
| GO:0035735 | Intraciliary transport | GO:0045597 | Positive regulation of cell |
| | involved in cilium | | differentiation* |
| | assembly** | | |
| GO:0051298 | Centrosome duplication* | GO:0019932 | Second-messenger- |
| | | | mediated signaling** |
| GO:0010639 | Negative regulation of | GO:0060284 | Regulation of cell |
| | organelle organization** | | development** |
| GO:0042769 | DNA damage response, | GO:0008015 | Blood circulation** |
| | detection of DNA | | |
| | damage** | | |
| GO:1901796 | Regulation of signal | GO:0030155 | Regulation of cell |
| | transduction by p53 class | | adhesion** |
| | mediator** | | |
| GO:0060964 | Regulation of gene | GO:0050920 | Regulation of chemotaxis** |
| | silencing by miRNA* | | |
| GO:0046605 | Regulation of centrosome | GO:0061061 | Muscle structure |
| | cycle* | | development** |
| GO:0006297 | Nucleotide-excision repair, | GO:0007409 | Axonogenesis* |
| | DNA gap filling* | | |
| GO:0006283 | Transcription-coupled | GO:0014706 | Striated muscle tissue |
| | nucleotide-excision | | development* |
| | repair* | | |
| GO:1901990 | Regulation of mitotic cell | GO:0045761 | Regulation of adenylate |
| | cycle phase transition* | | cyclase activity* |
| GO:0061512 | Protein localization to | GO:0022603 | Regulation of anatomical |
| | cilium* | | structure morphogenesis* |
| GO:0071459 | Protein localization to | GO:0001503 | Ossification* |
| | chromosome, centromeric | | |
| | region* | | |
| GO:0033047 | Regulation of mitotic | GO:0033627 | Cell adhesion mediated by |
| | sister chromatid | | integrin* |
| | segregation* | | |
| GO:0010948 | Negative regulation of cell | GO:0098742 | Cell-cell adhesion |
| | cycle process* | | viaplasma-membrane |
| | | | adhesion molecules* |
| GO:1902850 | Microtubule cytoskeleton | GO:0060348 | Bone development* |
| | organization involved in | | |
| | mitosis* | | |
| GO:0006334 | Nucleosome assembly* | GO:0003179 | Heart valve morphogenesis* |
| GO:0042770 | Signal transduction in | GO:0098698 | Postsynaptic specialization |
| | response to DNA damage* | | assembly* |
| GO:0051290 | Protein | GO:0090287 | Regulation of cellular |
| | heterotetramerization* | | response to growth factor |
| | | | stimulus* |
| GO:2001020 | Regulation of response to | GO:0042476 | Odontogenesis* |
| | DNA damage stimulus* | | |
| GO:0032259 | Methylation* | GO:0045860 | Positive regulation of |
| | | | protein kinase activity* |
| GO:0006303 | Double-strand break repair | GO:0006816 | Calcium ion transport* |
| | via nonhomologous end | | |
| | joining* | | |
| GO:0044839 | Cell cycle G2/M phase | GO:0051345 | Positive regulation of |
| | transition* | | hydrolase activity* |
| GO:0032201 | Telomere maintenance via | GO:0001822 | Kidney development* |
| | semi-conservative replication* | | |
| GO:0000077 | DNA damage checkpoint* | GO:0002009 | Morphogenesis of an |
| | | | epithelium* |
| GO:0051310 | Metaphase plate | GO:0007610 | Behavior* |
| | congression* | | |
| GO:0031297 | Replication fork | GO:1990869 | Cellular response to |
| | processing* | | chemokine* |
| GO:0000724 | Double-strand break repair | GO:0050877 | Nervous system process* |
| | via homologous | | |
| | recombination* | | |
| | | GO:0009968 | Negative regulation of |
| | | | signal transduction* |
| | | GO:0007167 | Enzyme linked receptor |
| | | | protein signaling pathway* |
| | | GO:0034220 | Ion transmembrane |
| | | | transport* |
| | | GO:0048645 | Animal organ formation* |
| | | GO:0003198 | Epithelial to mesenchymal |
| | | | transition involved in |
| | | | endocardial cushion |
| | | | formation* |
| | | GO:0030029 | Actin filament-based |
| | | | process* |
| | | GO:0030335 | Positive regulation of cell |
| | | | migration* |
| | | GO:0002062 | Chondrocyte |
| | | | differentiation* |
| | | GO:0071222 | Cellular response to |
| | | | lipopolysaccharide* |

TABLE 2
Functional enrichment of NPM1 relapse specific gene pairs segregated
into positive and negative co-expressions. All Gene Ontology
terms are significant (corrected p-value <0.05, FDR B&H).
Terms with corrected p-value <0.01 are marked with *,
and <0.001 are marked with**.

| Positive co-expression | | Negative co-expression | |
|---|---|---|---|
| GO ID | GO Name | GO ID | GO Name |
| GO:0098693 | Regulation | GO:0050911 | Detection of chemical |
| | of synaptic | | stimulus involved in |
| | vesicle | | sensory perception |
| | cycle* | | of smell** |
| GO:0097091 | Synaptic | GO:0032787 | Monocarboxylic acid |
| | vesicle | | metabolic process** |
| | clustering* | GO:0007186 | G protein-coupled receptor |
| | | | signaling pathway** |
| | | GO:0060333 | Interferon-gamma-Mediated |
| | | | signaling pathway** |
| | | GO:0045892 | Negative regulation of |
| | | | transcription, DNA |
| | | | templated* |
| | | GO:0050870 | Positive regulation of T cell |
| | | | activation* |
| | | GO:0044255 | Cellular lipid metabolic |
| | | | process* |
| | | GO:0042127 | Regulation of cell |
| | | | population proliferation* |
| | | GO:0050691 | Regulation of defense |
| | | | response to virus by host* |
| | | GO:0009719 | Response to endogenous |
| | | | stimulus* |
| | | GO:0042509 | Regulation of tyrosine |
| | | | phosphorylation of STAT |
| | | | protein* |
| | | GO:0044282 | Small molecule catabolic |
| | | | process* |
| | | GO:0034340 | Response to type I |
| | | | interferon* |
| | | GO:0032609 | Interferon-gamma |
| | | | production* |

The differential gene expression analysis shows that most upregulated genes in non-relapse HER2-positive breast cancer patients are involved in the immune system and cell cycle processes, while upregulated genes in relapse HER2-positive breast cancer patients are involved in proliferation, migration, angiogenesis, and anti-apoptosis. Genes involved in ossification are also upregulated in relapse HER2-positive breast cancer patients and this may be related to bone metastasis. The risk of leukemia increases in breast cancer survivors. Unanimously, genes (DNMT3A) that are leukemia-susceptible are upregulated in non-relapse HER2-positive breast cancer patients. Genes (EPOR & MAPT) that offer cardiovascular protections are unregulated in relapse HER2-positive breast cancer patients. However, these genes are associated with trastuzumab resistance.

### Example 3

### Chemo-Resistance of High Grade Serous Ovarian Cancer

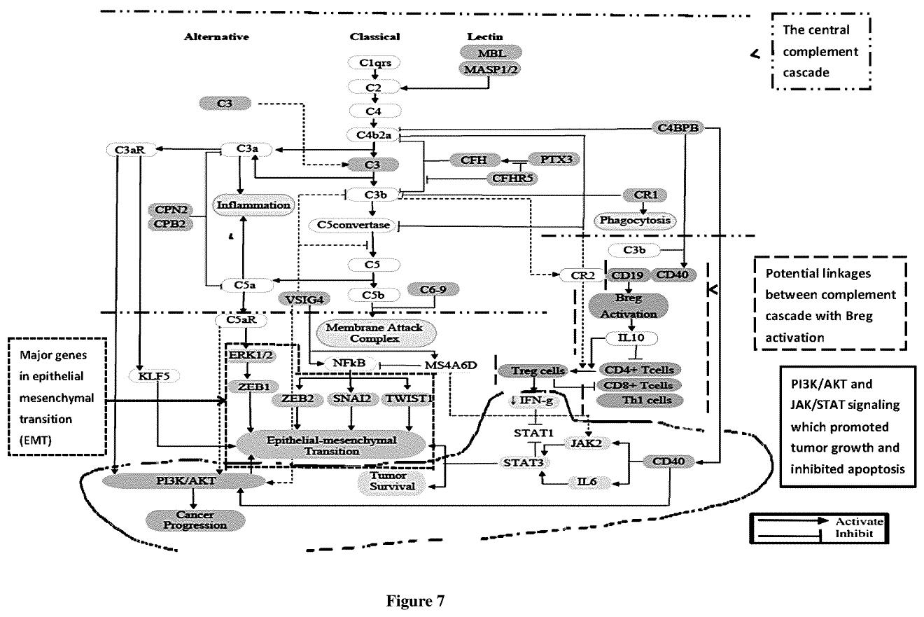

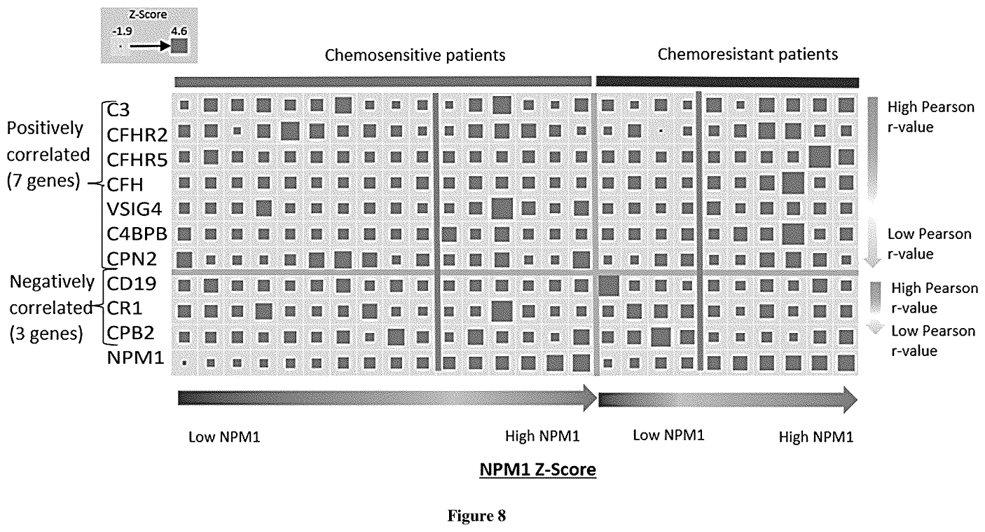

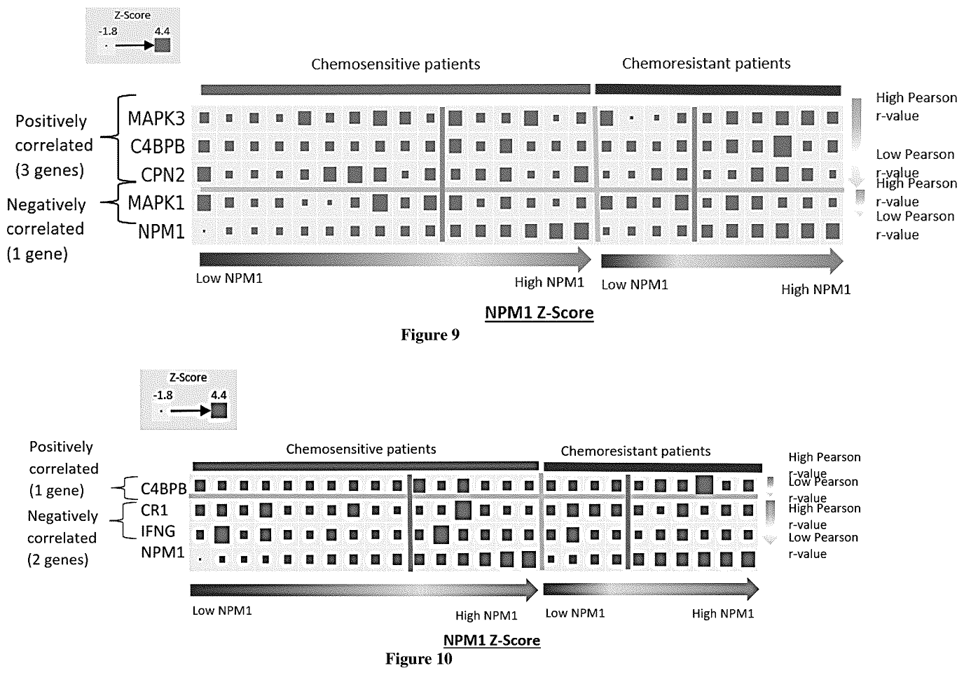

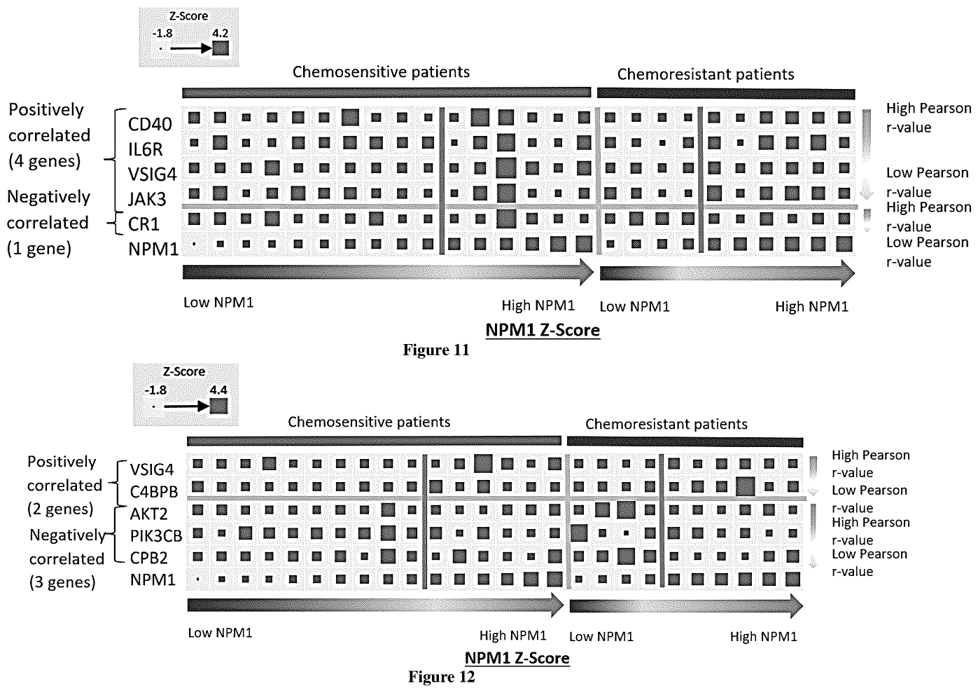

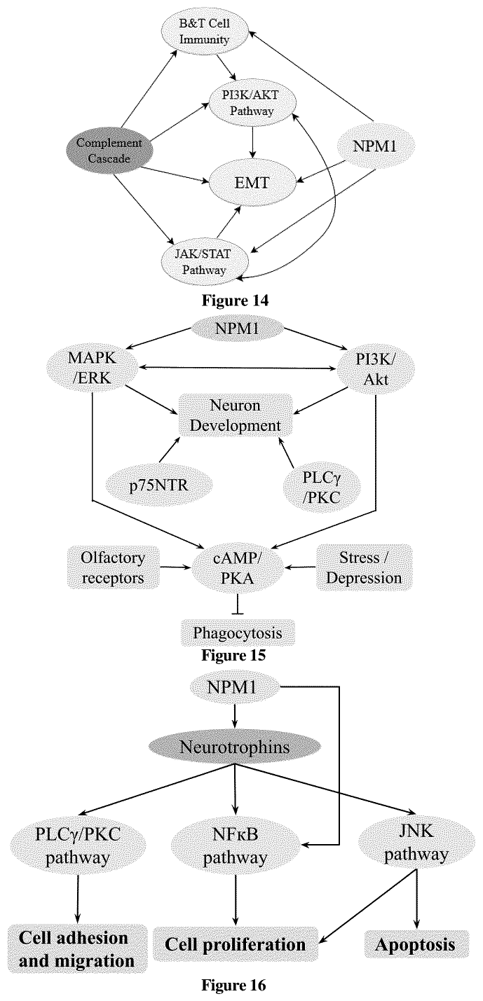

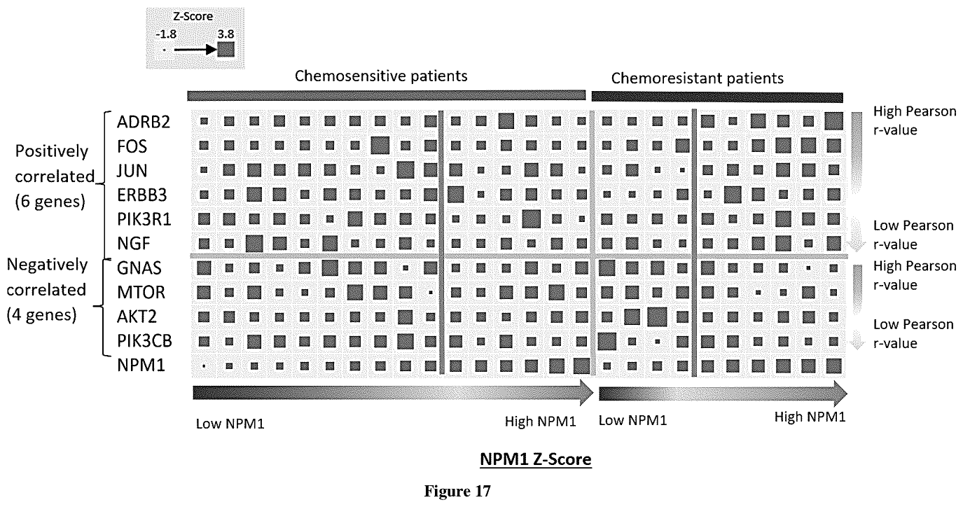

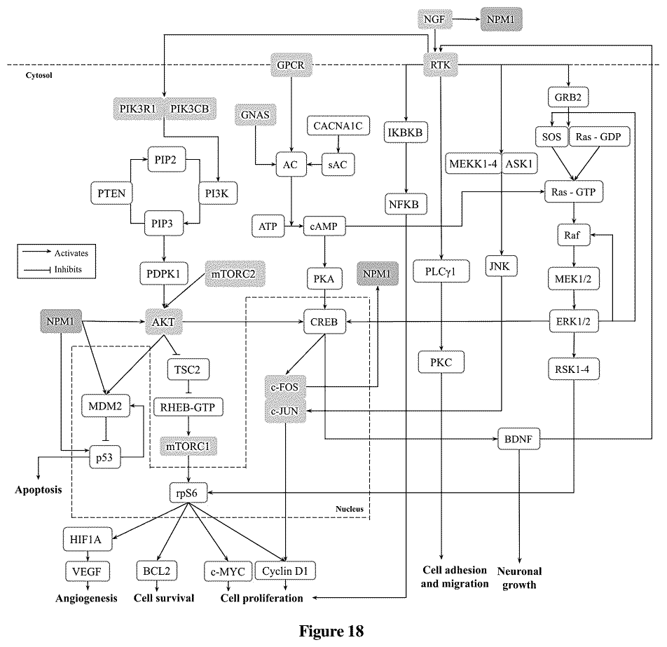

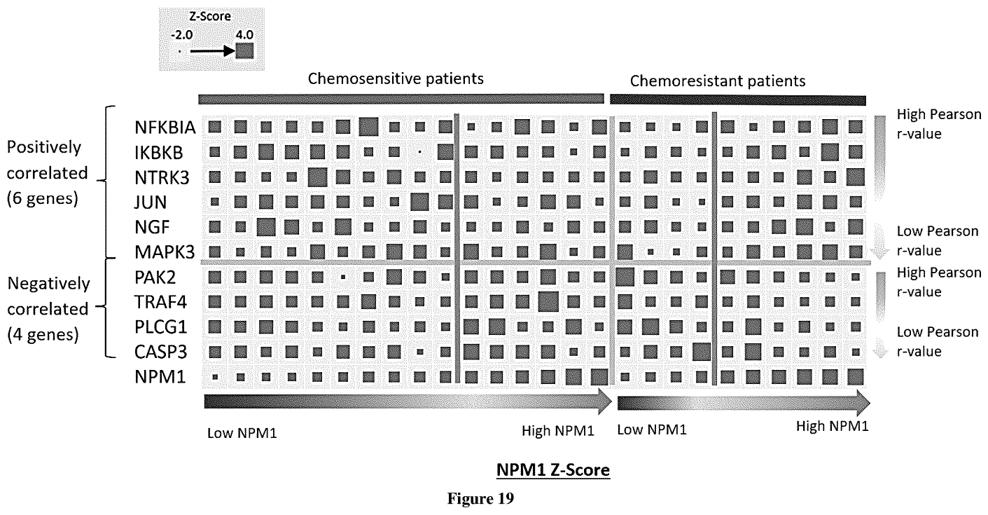

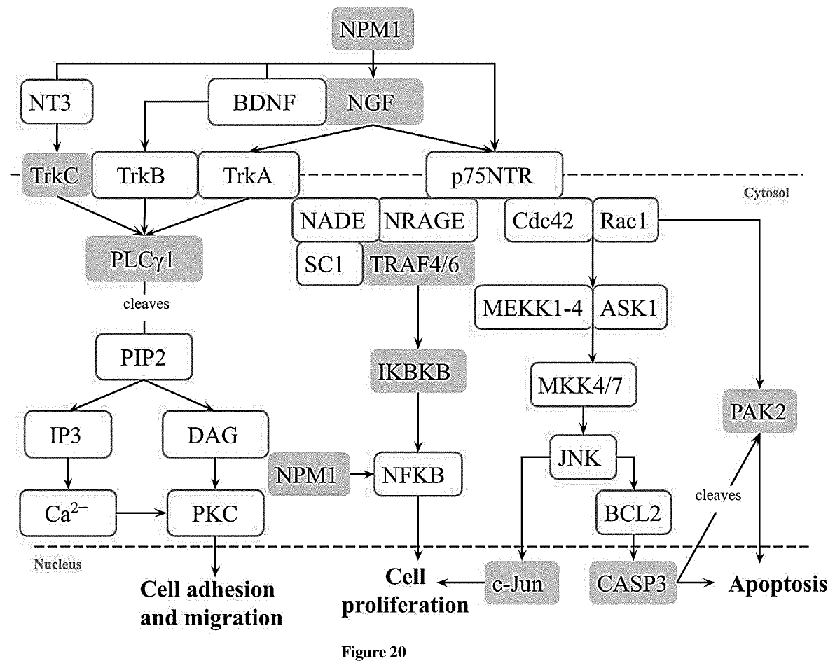

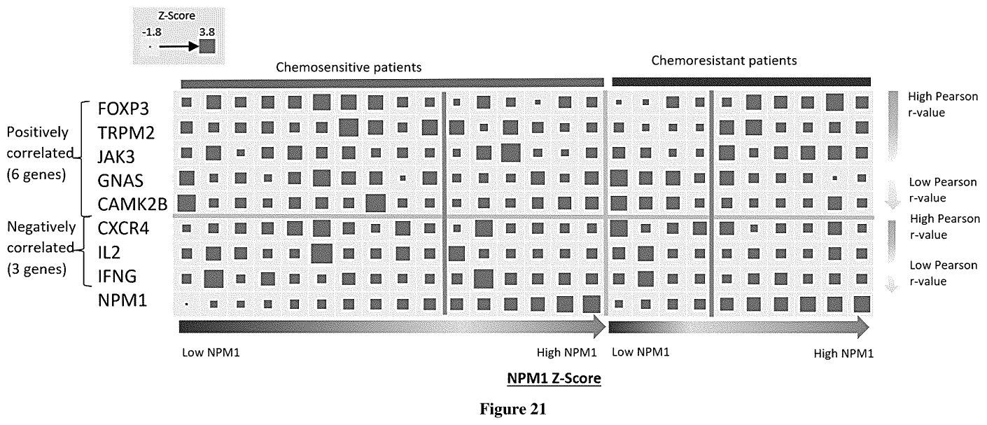

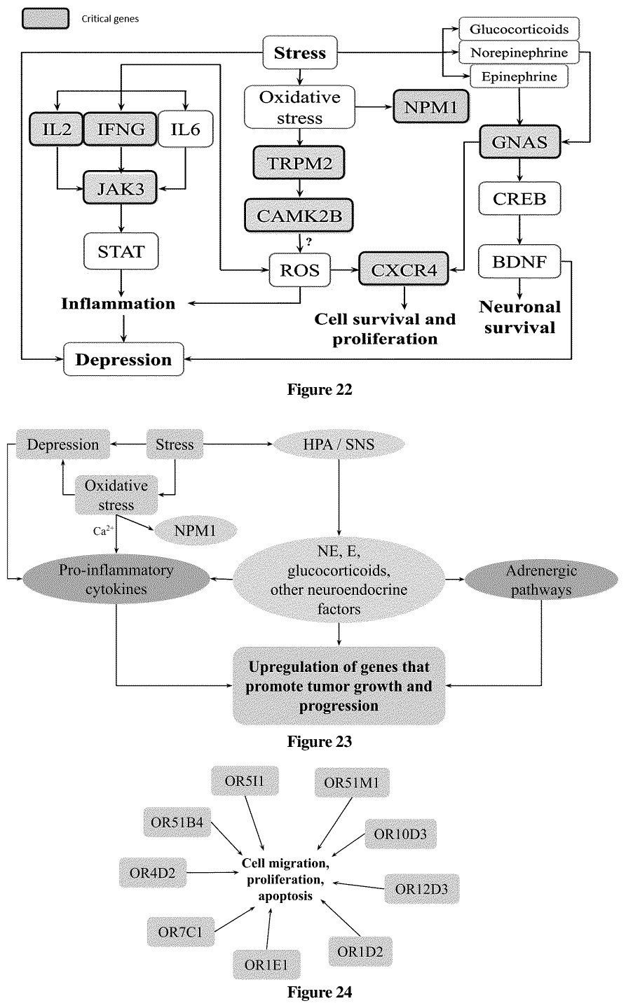

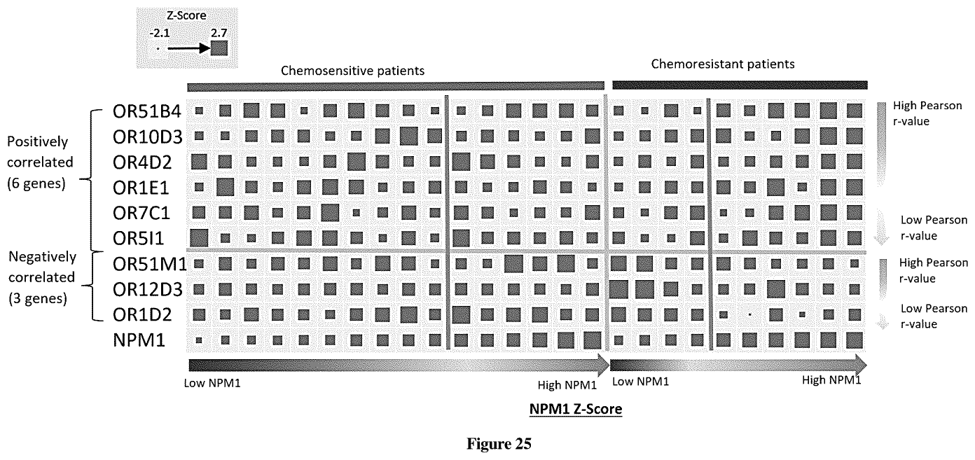

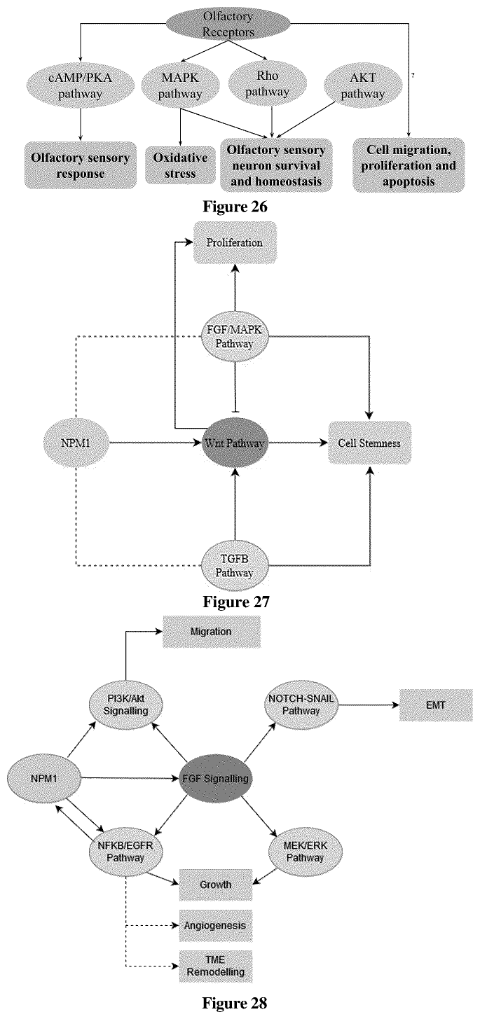

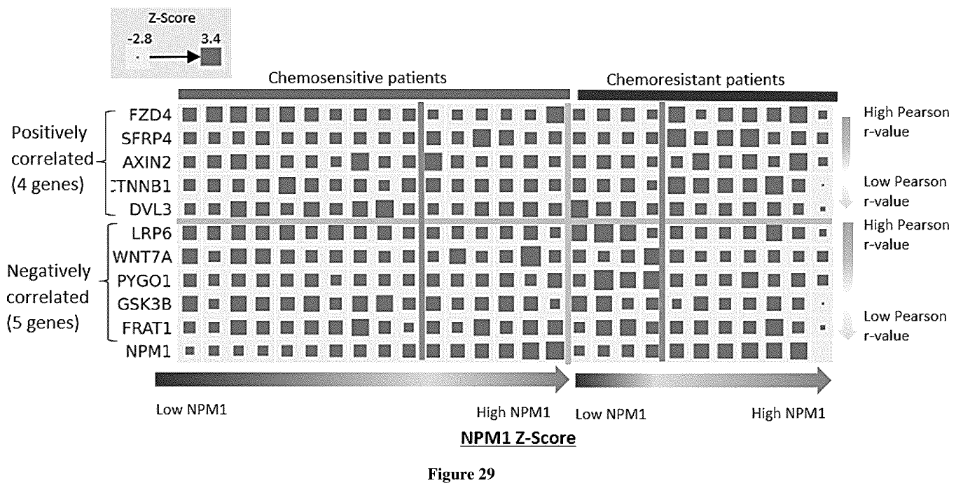

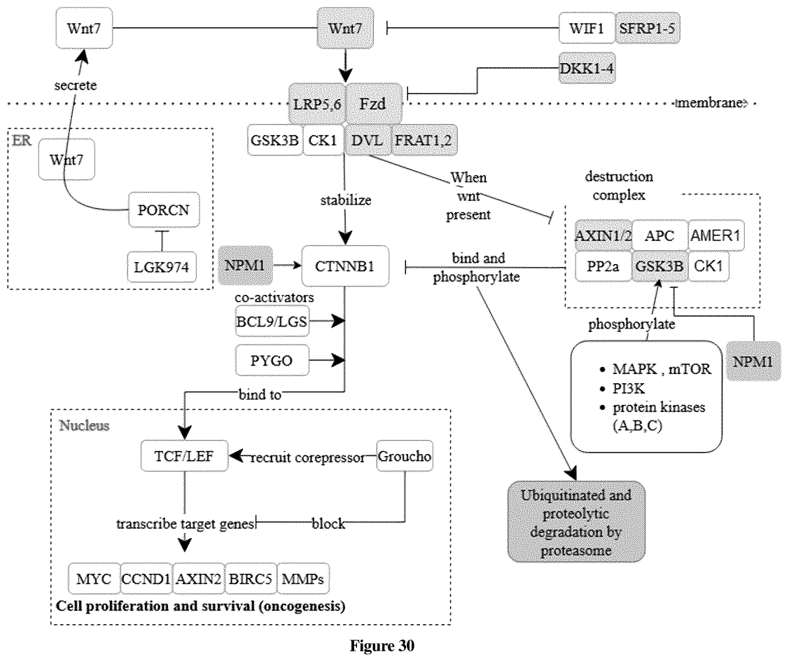

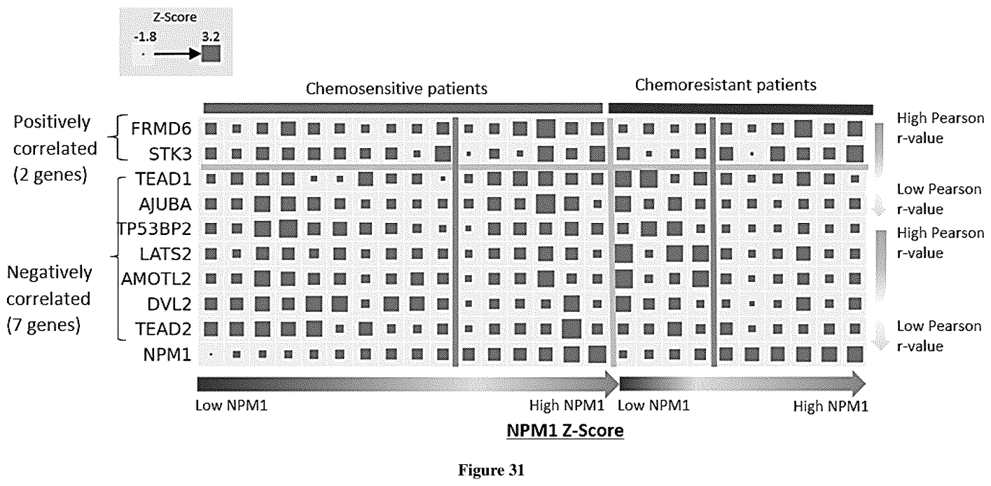

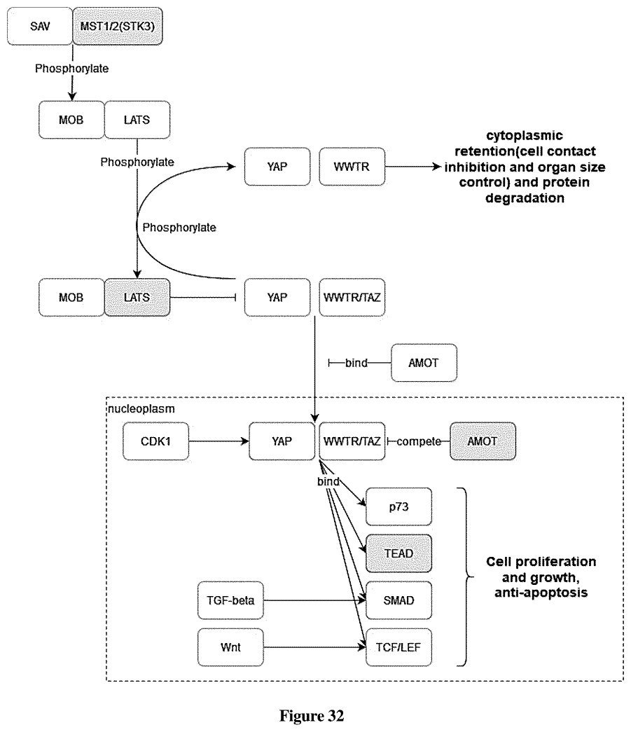

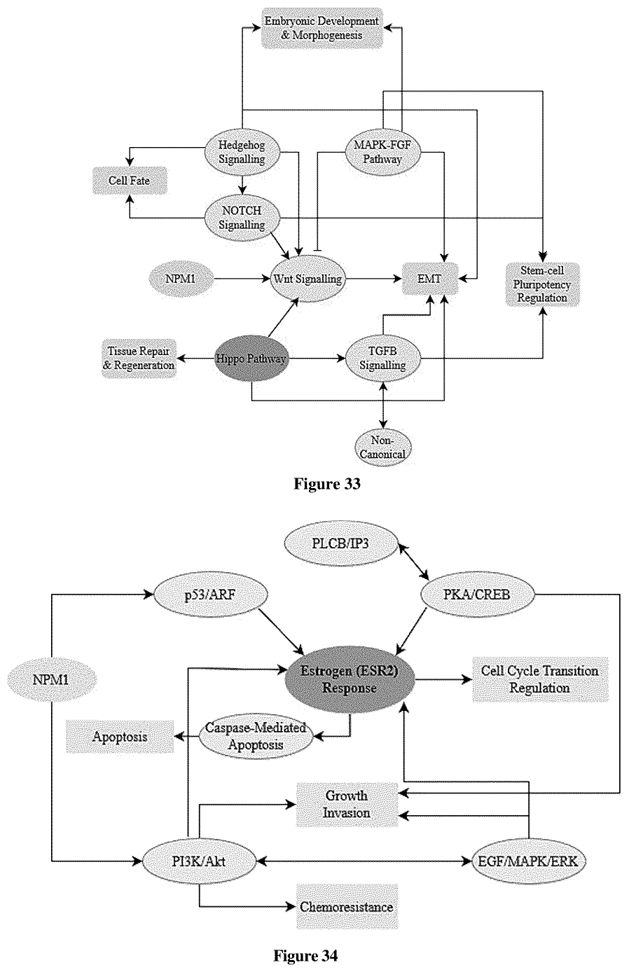

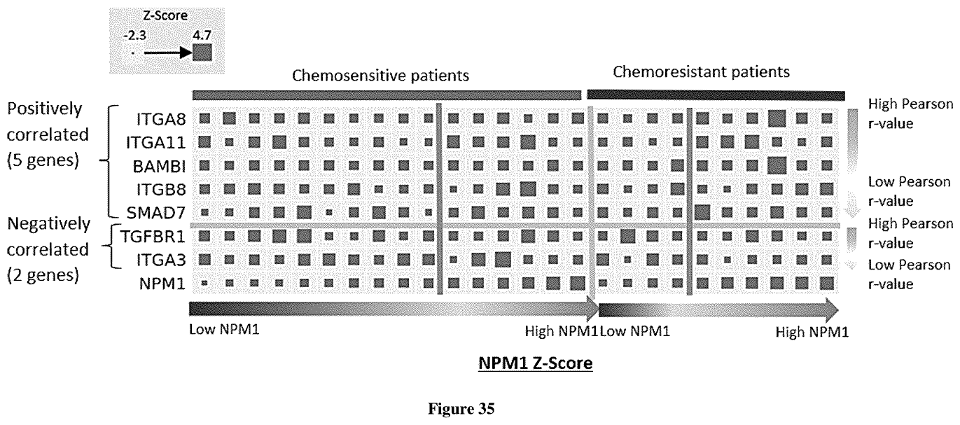

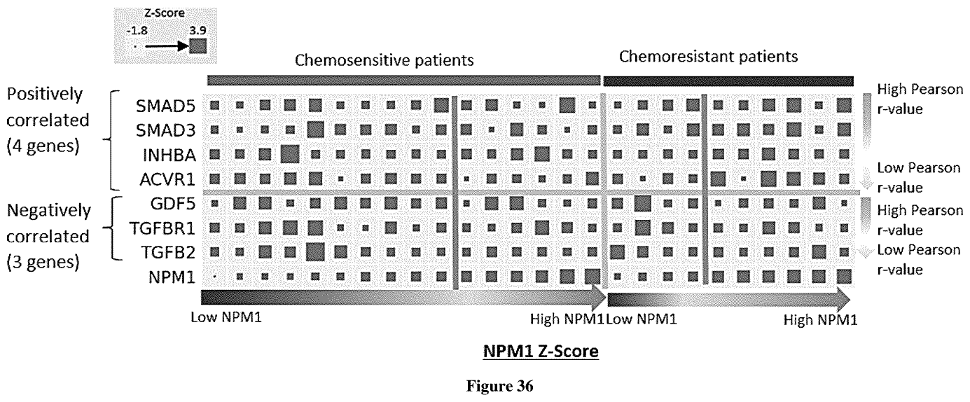

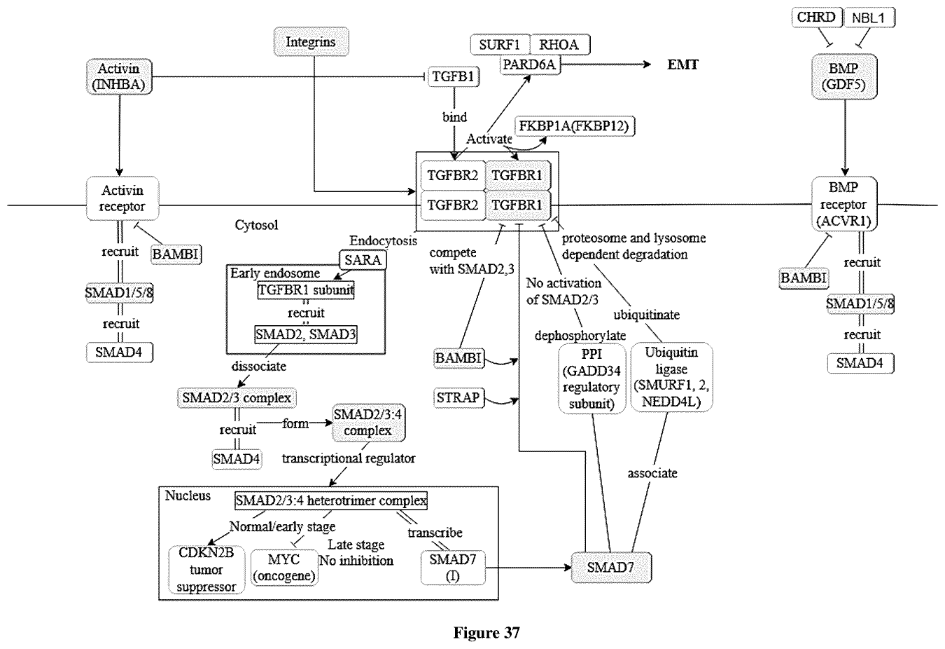

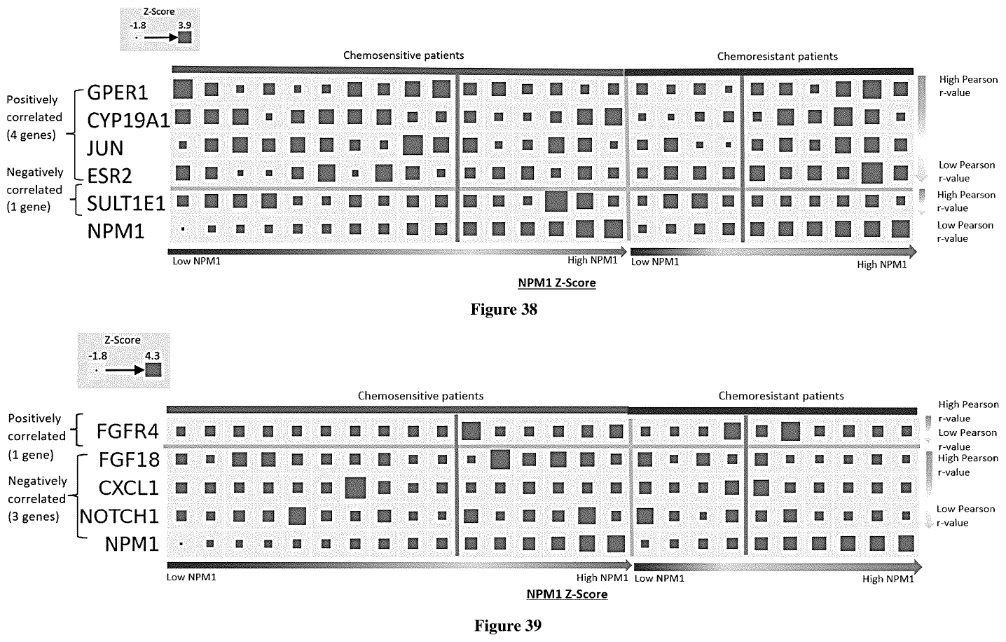

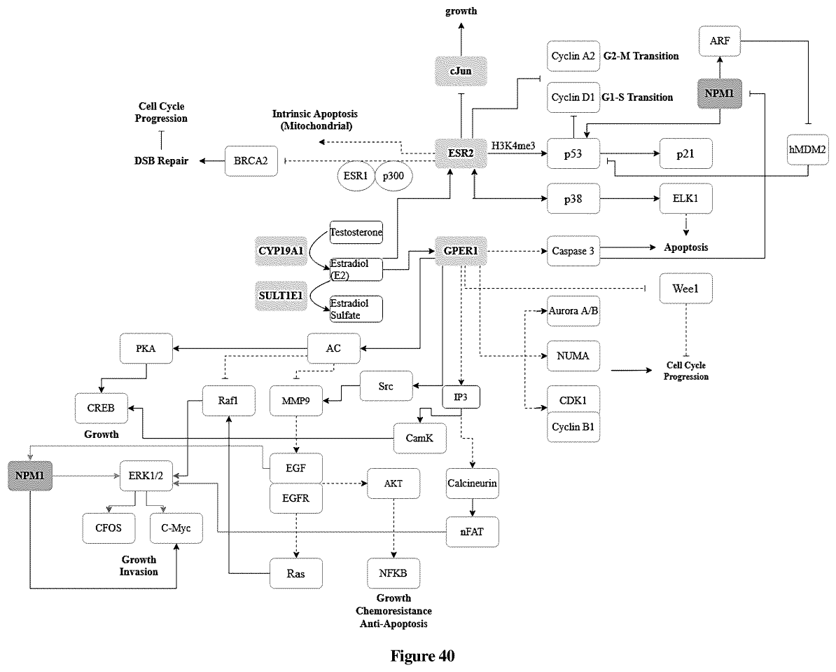

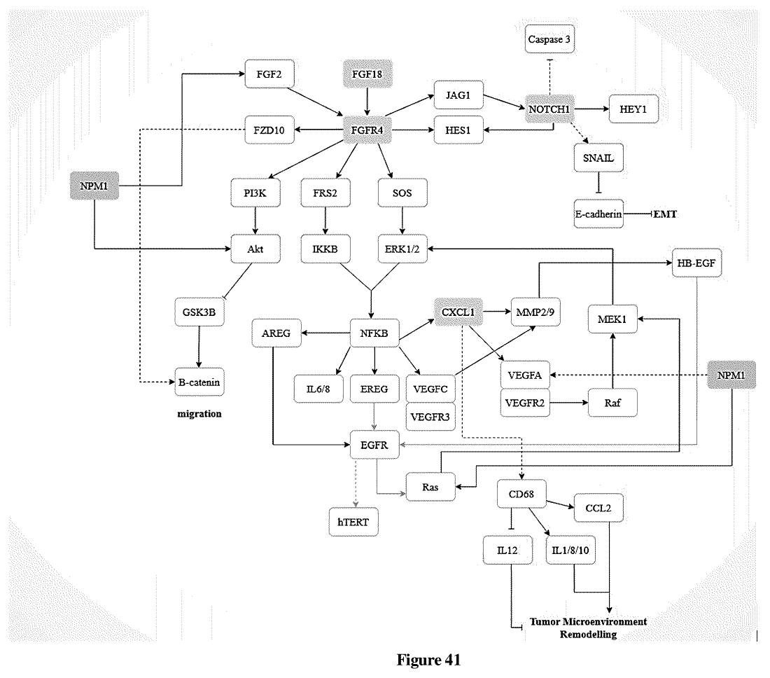

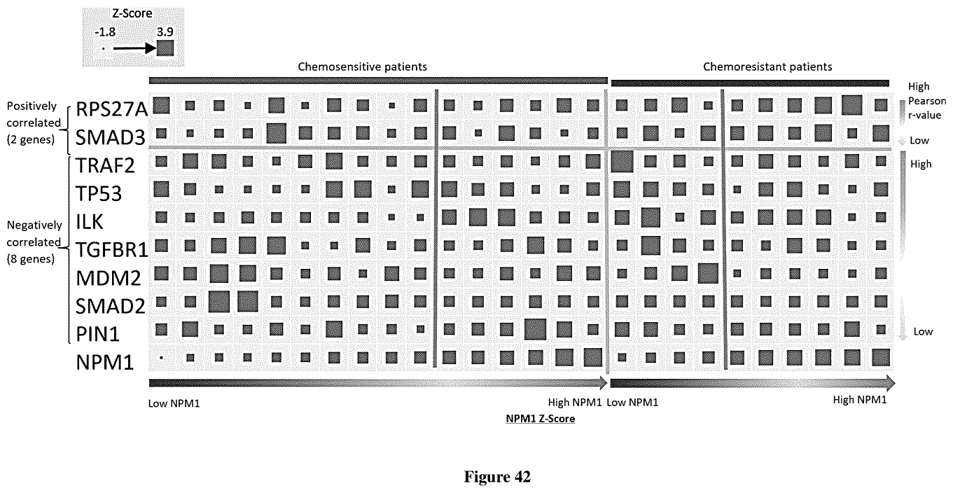

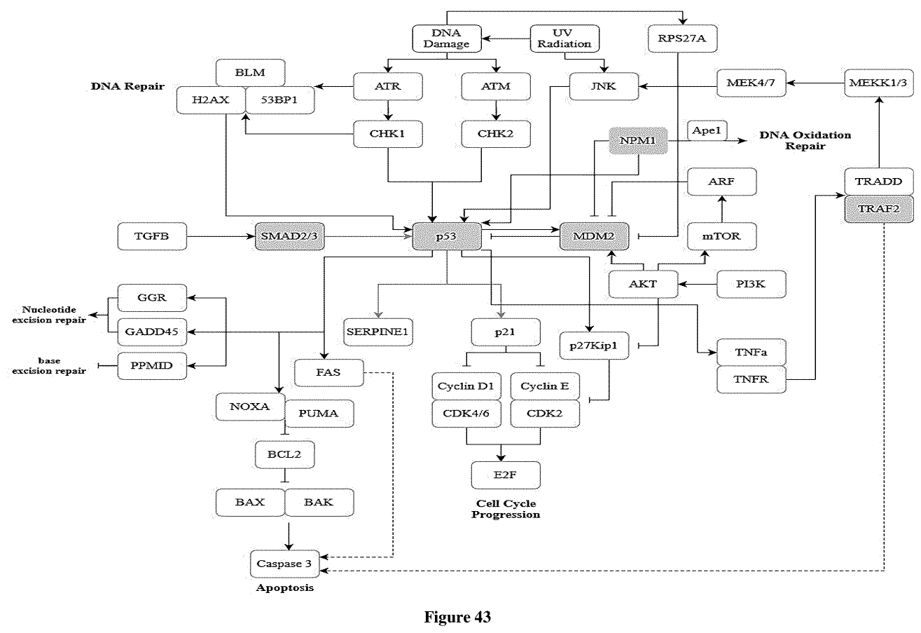

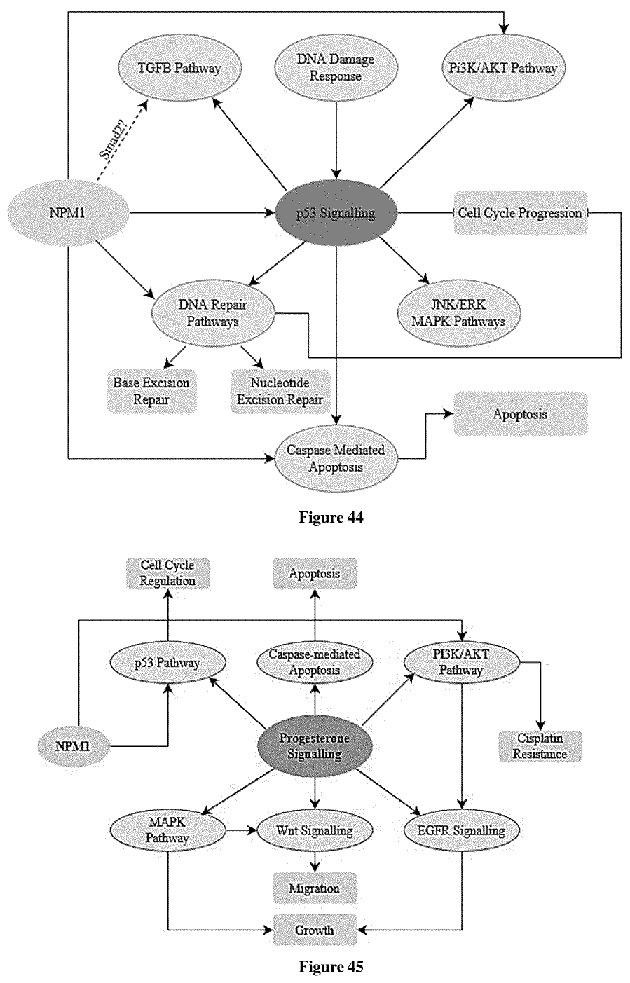

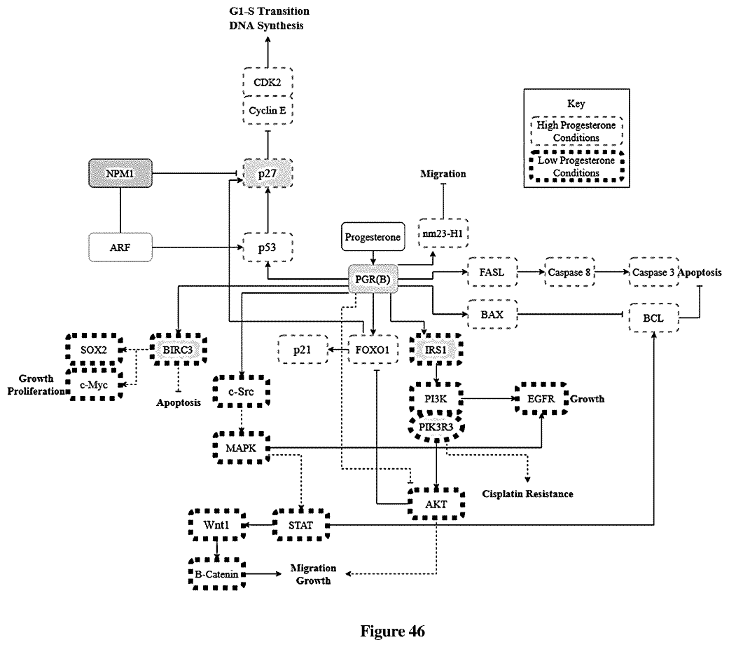

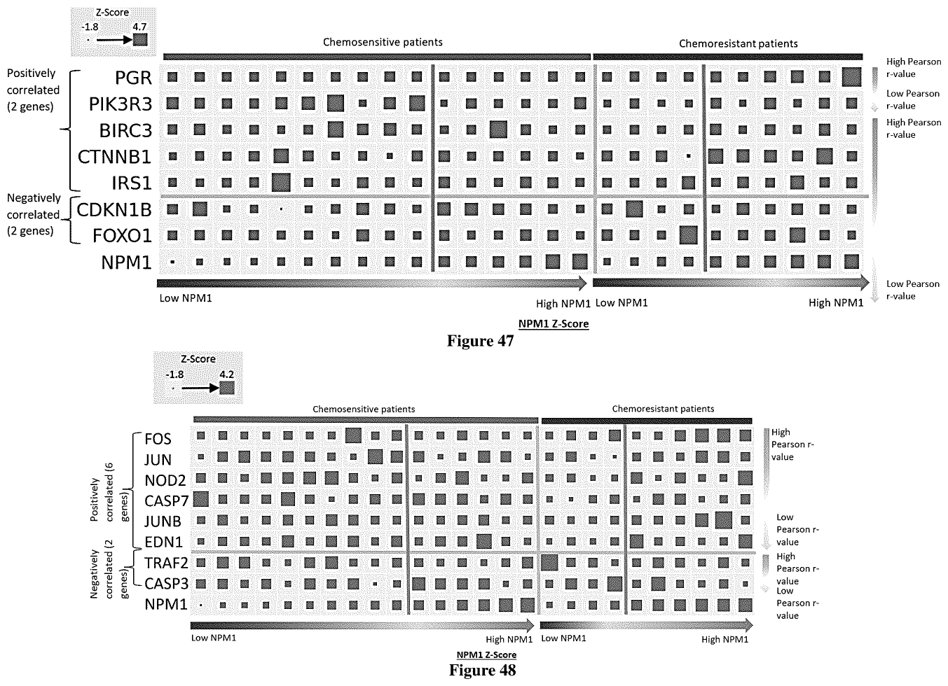

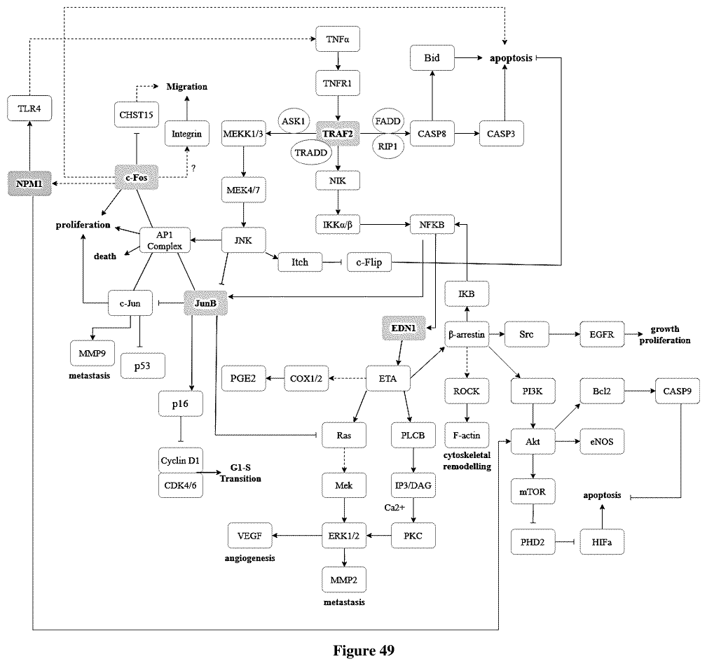

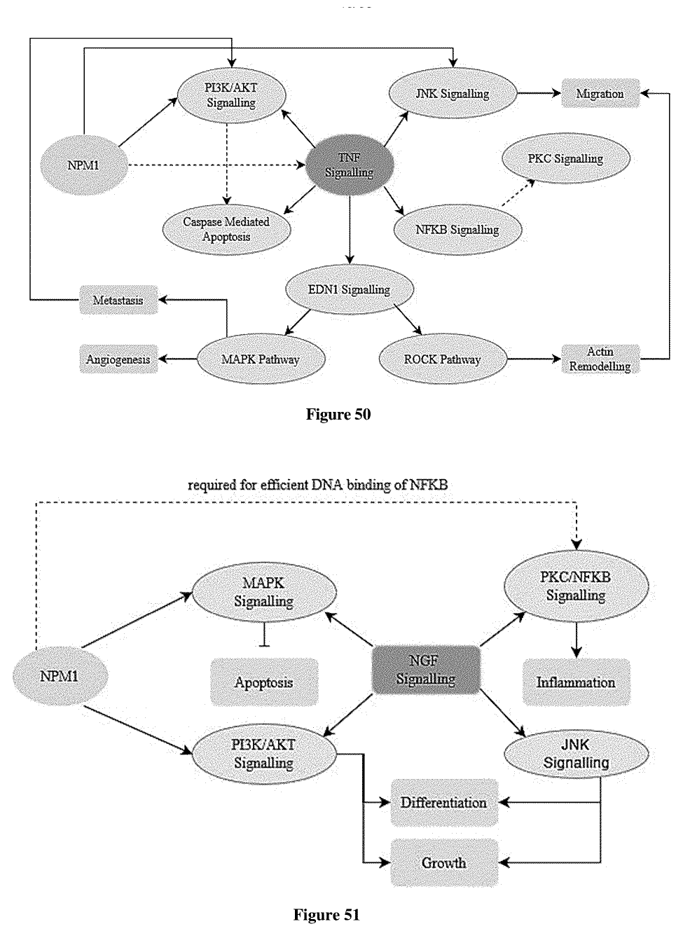

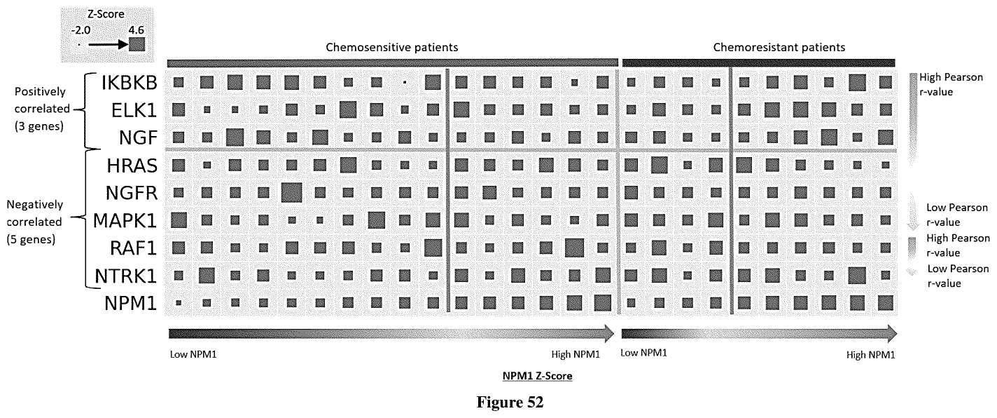

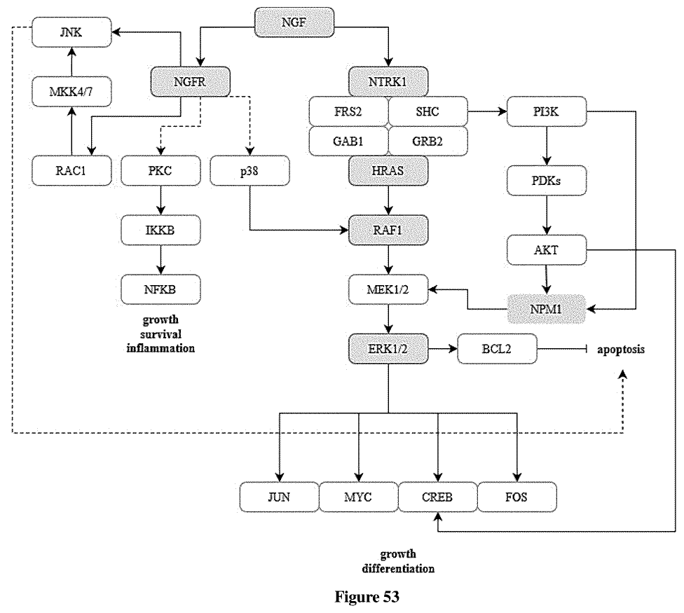

In one embodiment, this invention provides a method for diagnosing and predicting the chemo-resistance of high grade serous ovarian cancer (HGSOC) (FIGS. 2, 6, 7). A sample is first obtained from a subject and gene expression (RNAs) was analyzed with whole-genome co-expressional changes using methods such as Next Generation Sequencing and Microarray technologies or any other methods known in the art. Our method was then used for conducting i) NPM1 Structural Gene Co-expression Analysis & Functional and Pathway Enrichment (Domain 1: Complement Cascade): NPM1 correlated Genes (C4BPB, CD19, CFHR5, CPB2, CR1, VSIG4) in the Complement Cascade Module (FIG. 8), NPM1 correlated Genes (AKT2, PIK3CB, C4BPB, CPB2, VSIG4) in the PI3K/AKT Module (FIG. 12), NPM1 correlated Genes (JAK3, CR1, IL6R, IL6, CD40, VSIG4) in the JAK/STAT Module (FIG. 11), NPM1 correlated Genes (C4BPB, CPN2, MAPK1, MAPK3) in the Epithelial-mesenchymal transition (EMT) Module (FIG. 9), NPM1 correlated Genes (IFNG, CR1, C4BPB) in the Adaptive Immunity Module (FIG. 10); ii) NPM1 Structural Gene Co-expression Analysis & Functional and Pathway Enrichment (Domain 2: Tissue Development): NPM1 correlated Genes (FZD4, SFRP4, AXIN2, CTNNB1, DVL3, LRP6, WNT7A, PYGO1, GSK3B, FRAT1) in the Wnt Signaling Module (FIGS. 27, 29, 30), NPM1 correlated Genes (TGFBR1, TGFB2, SMAD5, INHBA, GDF5) in the TGFB signaling Module, NPM1 correlated Genes (ITGA8, ITGA3, ITGB8, SMAD7, TGFBR1) in the TGFB-ITG signaling Module (FIGS. 35, 36, 37), NPM1 correlated Genes (STK3, TEAD1, LATS2, AMOTL2, TEAD2) in the Hippo Pathway Module (FIGS. 31, 32, 33); iii) NPM1 Structural Gene Co-expression Analysis & Functional and Pathway Enrichment (Domain 3: Cellular Response to Growth Factors): NPM1 correlated Genes (ESR2, SULT1E1, GPER1, CYP19A1, JUN) in the Estrogen Module (FIGS. 34, 38, 40), NPM1 correlated Genes (FGF18, FGFR4, NOTCH1, CXCL1) in the Fibroblast Growth Factor (FGF) Module (FIGS. 28, 39, 41), NPM1 correlated Genes (TP53, MDM2, TRAF2, SMAD2) in the P53 response Module (FIGS. 42, 43, 44), NPM1 correlated Genes (FOS, JUNB, TRAF2, EDN1) in the Tumor Necrosis Factor (TNF) Module (FIGS. 48, 49, 50), NPM1 correlated Genes (PGR, PIK3R3, IRS1, CDKN1B, BIRC3) in the Progesterone Module (FIGS. 45, 46, 47), NPM1 correlated Genes (NGF, NGFR, NTRK1, MAPK1, HRAS, RAF1) in the Nerve Growth Factor (NGF) Module (FIGS. 51, 52, 53), NPM1 correlated Genes (WASL, MYH11, MYH14, PAK2, ACTN4, CLTCL1) in the Ephrins Module; iv) NPM1 Structural Gene Co-expression Analysis & Functional and Pathway Enrichment (Domain 4: Sensory Development): NPM1 correlated Genes (ADRB2, MTOR, PIK3R1, AKT2, PIK3CB, JUN, ERBB3) in the General Module (FIGS. 15, 17, 18), NPM1 correlated Genes (IKBKB, NTRK3, JUN, NGF, TRAF4, PLCG1, CASP3, PAK2) in the Neuron Development Module (FIGS. 16, 19, 20), NPM1 correlated Genes (GNAS, IL2, IFNG, JAK3, CAMK2B) in the Neuroendocrine Module (FIGS. 21, 22, 23), NPM1 correlated Genes (OR51M1, OR51B4, OR7C1) in the Olfactory Module (FIGS. 24, 25, 26),

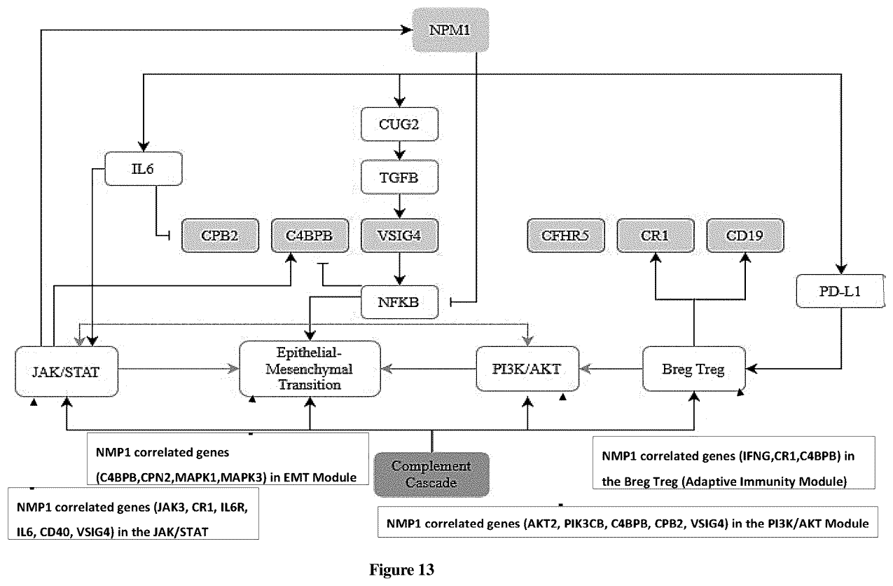

The Microscopic view of the interconnected network of Complement Cascade, Epithelial-mesenchymal transition (EMT), Adaptive Immunity, JAK/STAT, and PI3K/AKT are shown in FIG. 13; The Macroscopic view of the interconnected network of Complement Cascade, Epithelial-mesenchymal transition (EMT), Adaptive Immunity, JAK/STAT, and PI3K/AKT modules that contributes to the Chemo-resistance of HGSOC is shown in FIG. 14. Table B shows the conditions used in this Example.

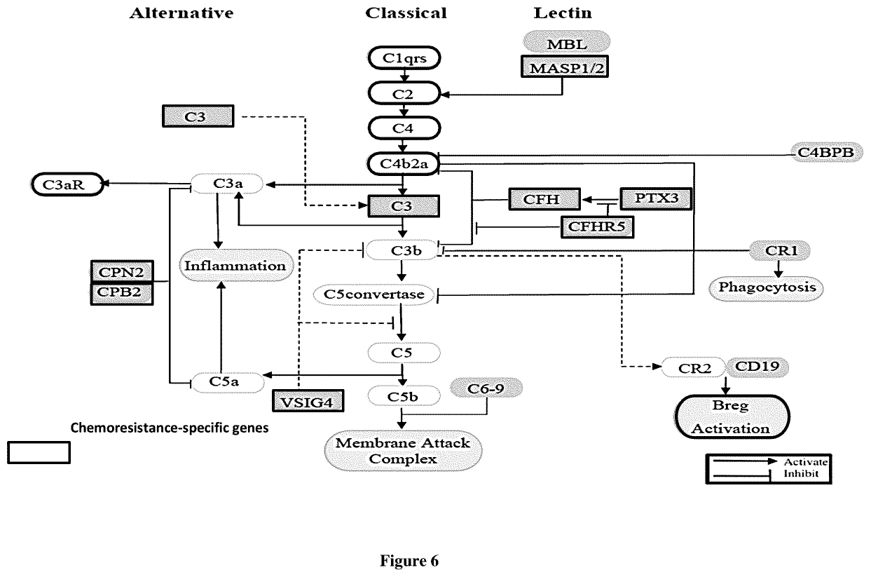

TABLE B
Conditions used in Example 3

| Parameter | | Condition |
|---|---|---|
| Genetic trait | | Chemoresistance |
| State of interest | | high grade serous ovarian cancer |
| | | cells under question |
| Reference state | | high grade serous ovarian cancer |
| | | with chemoresistance |
| Gene of interest | | NPM1-correlated genes |
| | | appearing in FIGS. 6-53 |
| Methods | First gene | Data of cancer patients with |
| | expression data | chemoresistance were obtained |
| | | on online databases, with |
| | | pre-categorized data to show |
| | | patients' chemosensitivity state |
| | Second gene | Data of cancer patients exhibiting |
| | expression data | chemosensitivity were obtained |
| | | on online databases, with |
| | | pre-categorized data to show |
| | | patients' chemosensitivity state |
| | First co-expression | NPM1 co-expression analysis |
| | analysis | with MATLAB |
| | Second co-expression | NPM1 co-expression analysis with |
| | analysis | MATLAB |
| | Differential expression | N/A |
| | analysis | |
| | Connectivity | STRING |
| | functional enrichment or | ClueGO, KEGG, Reactome, |
| | pathway enrichment | PathCards |

### Example 4

### Reoccurrence of Colorectal Cancer

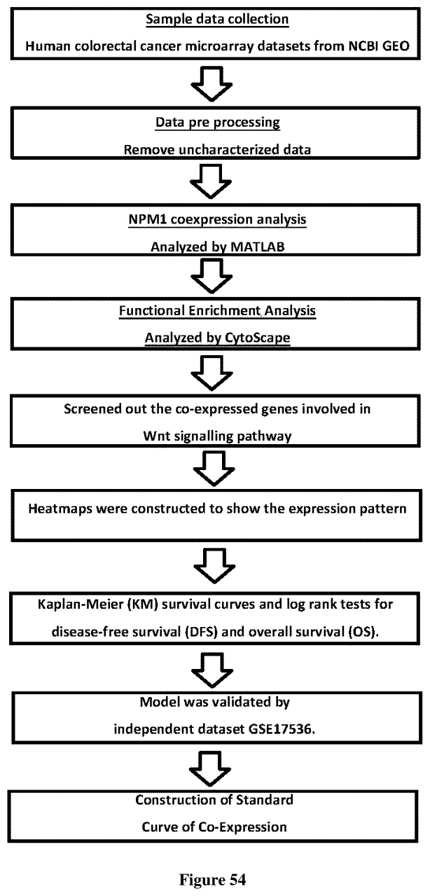

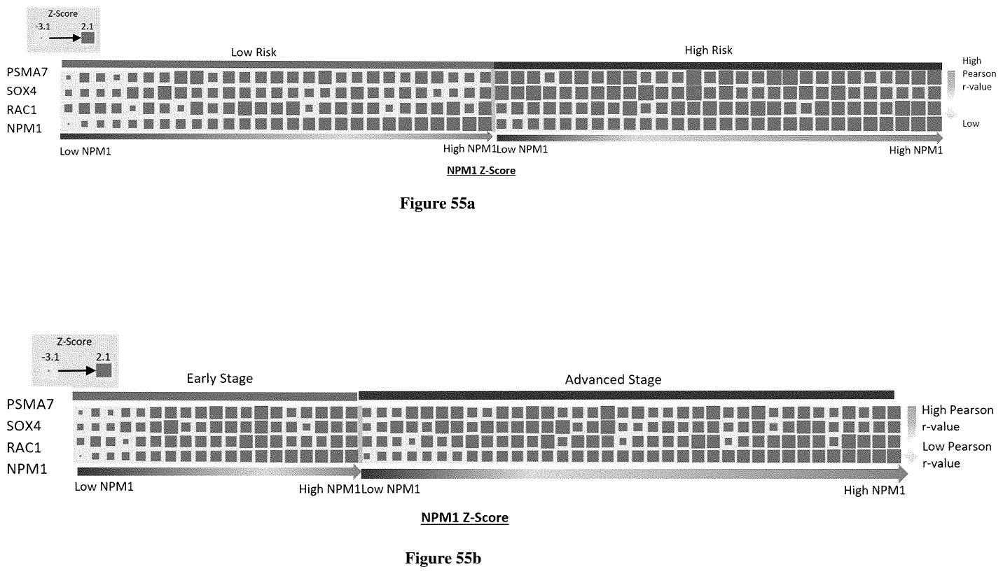

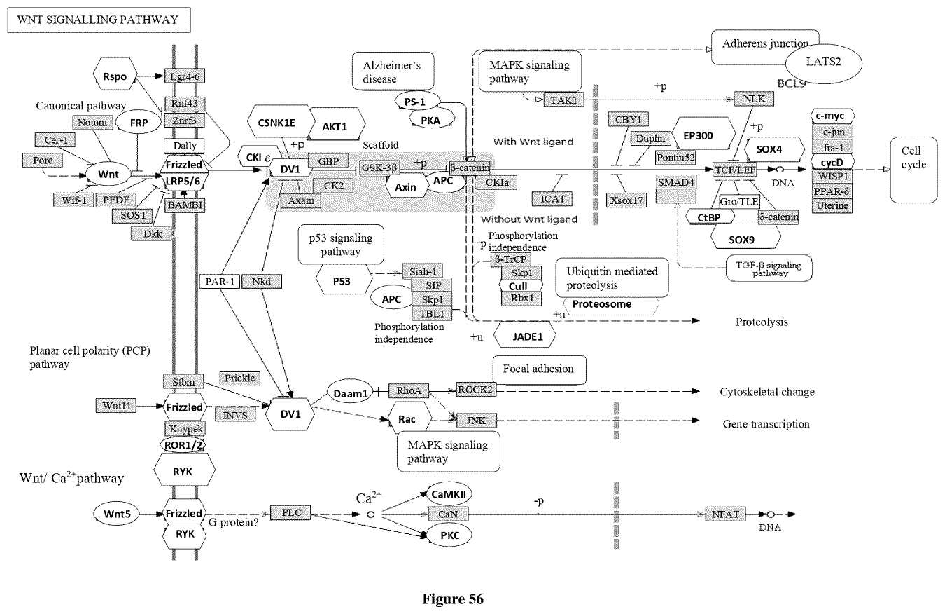

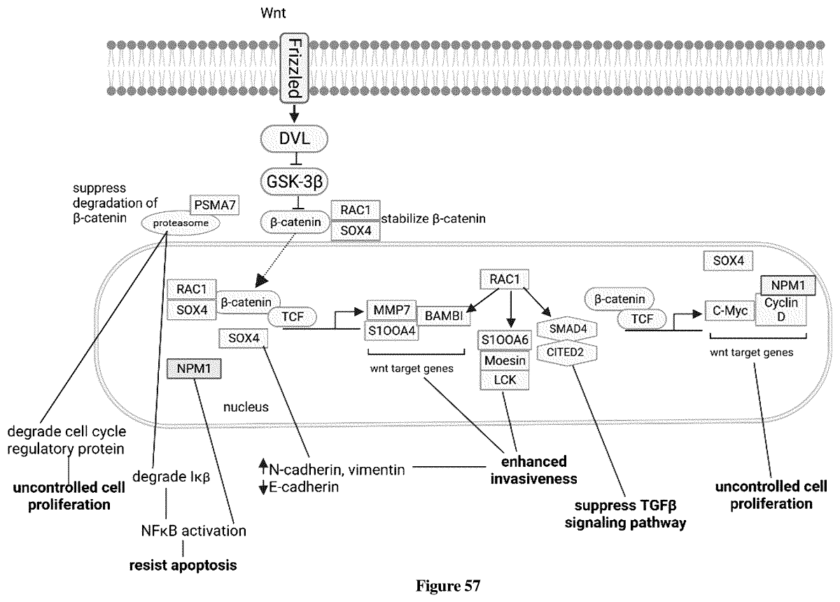

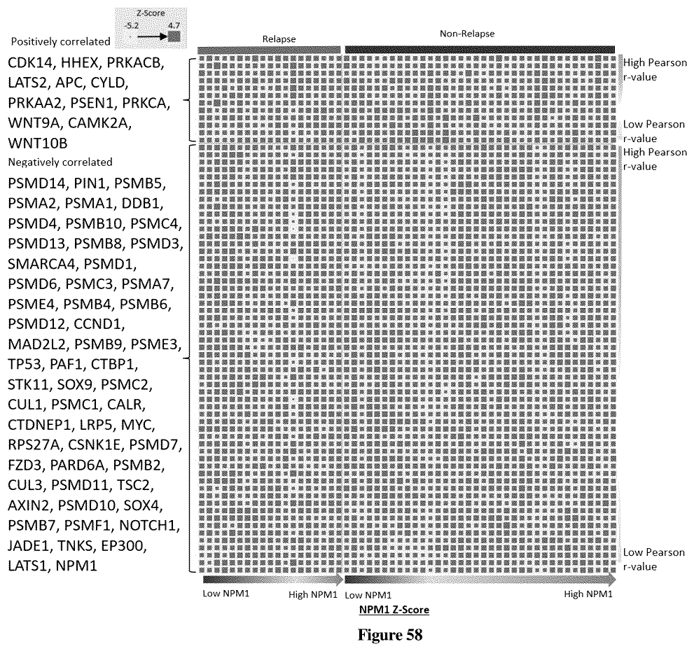

In one embodiment, this invention provides a method (FIG. 54) for diagnosing and predicting the reoccurrence of colorectal cancer. A sample is first obtained from a subject and gene expression (RNAs) was analyzed with whole-genome co-expressional changes using methods such as Next Generation Sequencing and Microarray technologies or any other methods known in the art. Our method was then used for conducting NPM1 Structural Gene Co-expression Analysis & Functional and Pathway Enrichment: NPM1 correlated Genes (PSMA7, SOX4 & Rac1) in the Wnt signaling pathway (FIGS. 55a, 55b, 56, 57, 58). (Tables 3-5). Table C shows the conditions used in this Example.

TABLE C
Conditions used in Example 4

| Parameter | | Condition |
|---|---|---|
| Genetic trait | | Cancer reoccurrence |
| State of interest | | Colorectal cells under question |
| Reference state | | Colorectal cells known to reoccur |
| Gene of interest | | NPM1-correlated genes appearing in |
| | | FIGS. 55a, 55b, 56, 57, 58 |
| Methods | First gene | Data of cancer patients with recurrence |
| | expression data | were obtained on online databases, |
| | | with pre-recurrence state |
| | Second gene | Data of cancer patients without |
| | expression data | recurrence were obtained on online |
| | | databases, with pre-categorized |
| | | data to show patients' |
| | | recurrence state |
| | First co-expression | NPM1 co-expression analysis with |
| | analysis | MATLAB |
| | Second co-expression | NPM1 co-expression analysis with |
| | analysis | MATLAB |
| | Differential expression | N/A |
| | analysis | |
| | Connectivity | STRING |
| | functional enrichment | ClueGO, KEGG |
| | or pathway enrichment | |

TABLE 3
Genes exhibited significant expression between relapse and
non-relapse patients when NPM1 expressed high. (p < 0.05)

| Genes | p-value |
|---|---|
| CTBP1 | 0.005 |
| PSMB2 | 0.017 |
| PSMD10 | 0.027 |
| RPS6KB2 | 0.032 |
| MAD2L2 | 0.041 |
| PSMB8 | 0.049 |

TABLE 4
Genes exhibited significant expression between relapse and non-
relapse patients when NPM1 expressed low. (p < 0.05)

| Genes | p-value |
|---|---|
| PSMD14 | 0.018 |
| PSMD1 | 0.002 |
| PIN1 | 0.005 |
| PSMA2 | 0.007 |
| PSMD4 | 0.008 |
| SMARCA4 | 0.011 |
| PSME4 | 0.016 |
| PSMB4 | 0.022 |
| PSMB5 | 0.022 |
| CALR | 0.022 |
| CTDNEP1 | 0.023 |
| Rac1 | 0.025 |
| PSMA7 | 0.026 |
| SOX4 | 0.040 |

TABLE 5
Genes exhibited significant expression
between relapse and non-relapse patients

| Gene | p-value |
|---|---|
| PSMA7 | 0.012 |
| SOX4 | 0.011 |
| Rac1 | 0.045 |

### Example 5

### Staging of Lung Adenocarcinoma

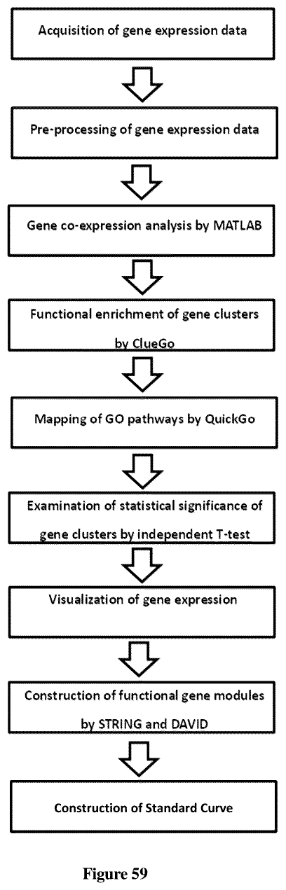

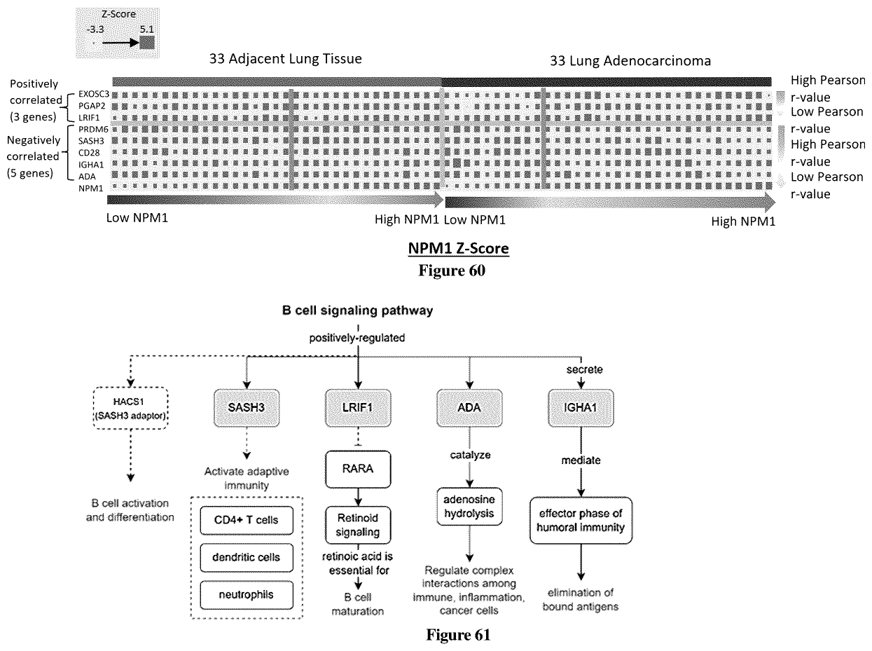

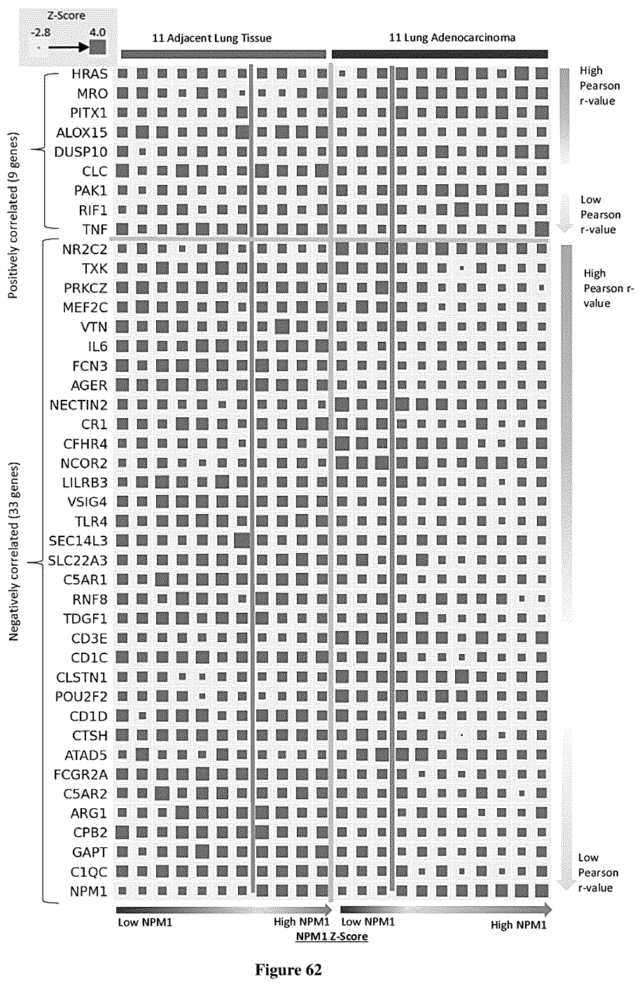

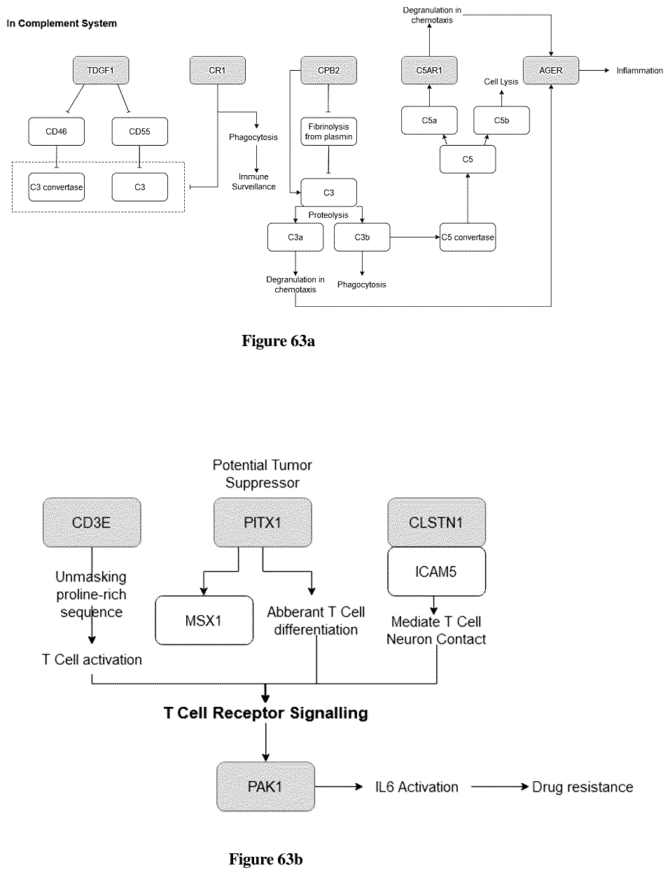

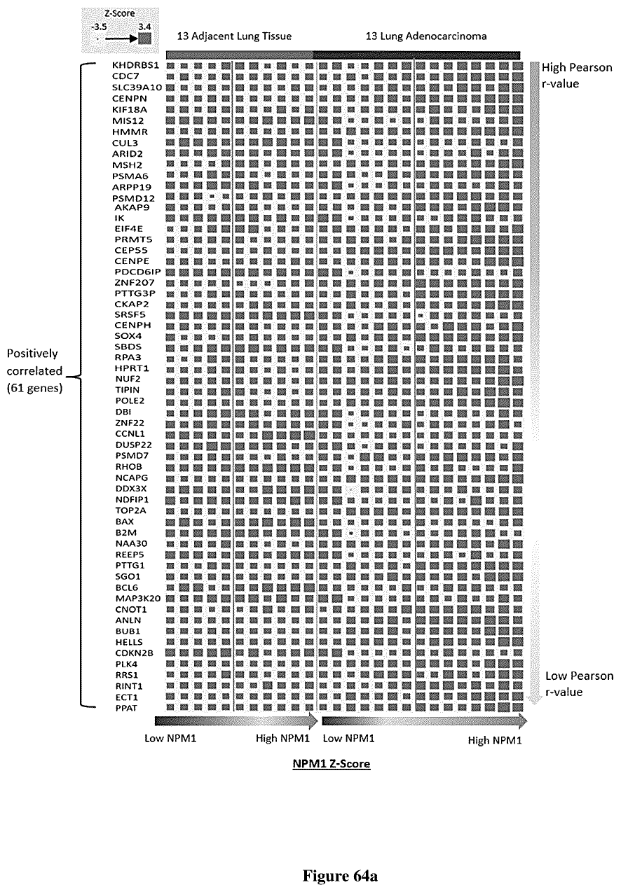

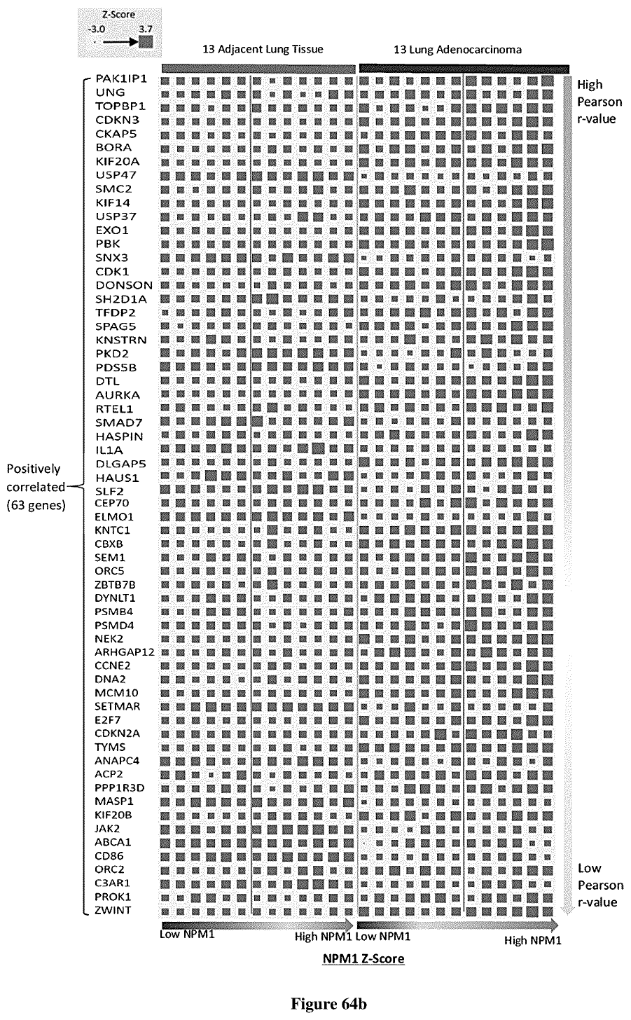

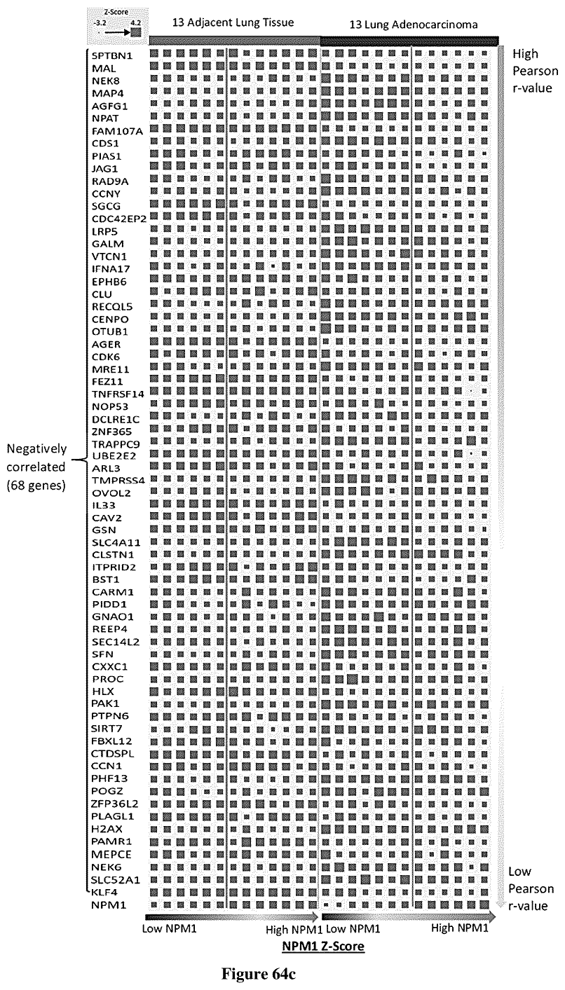

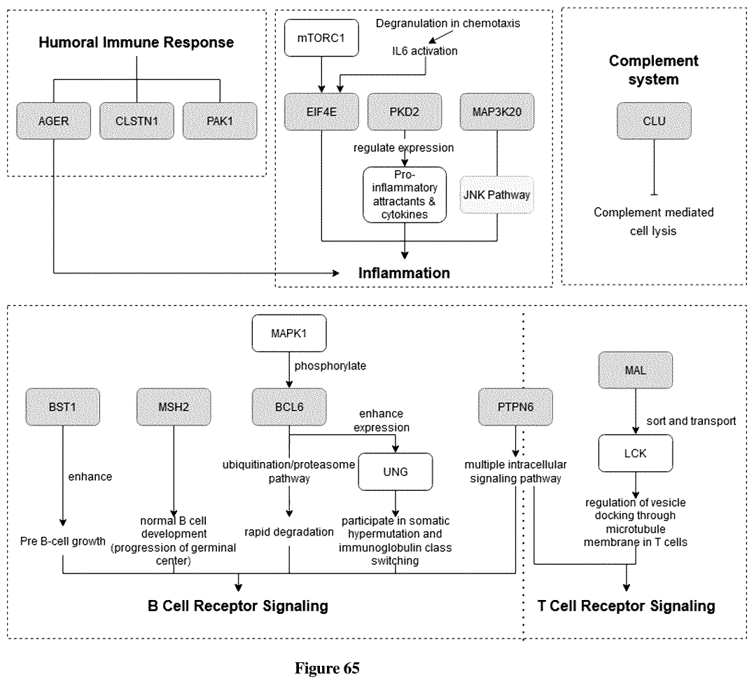

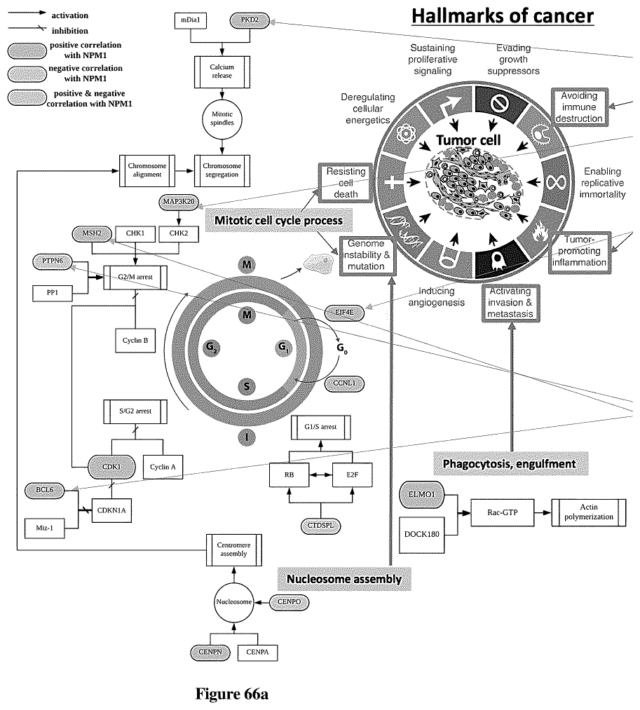

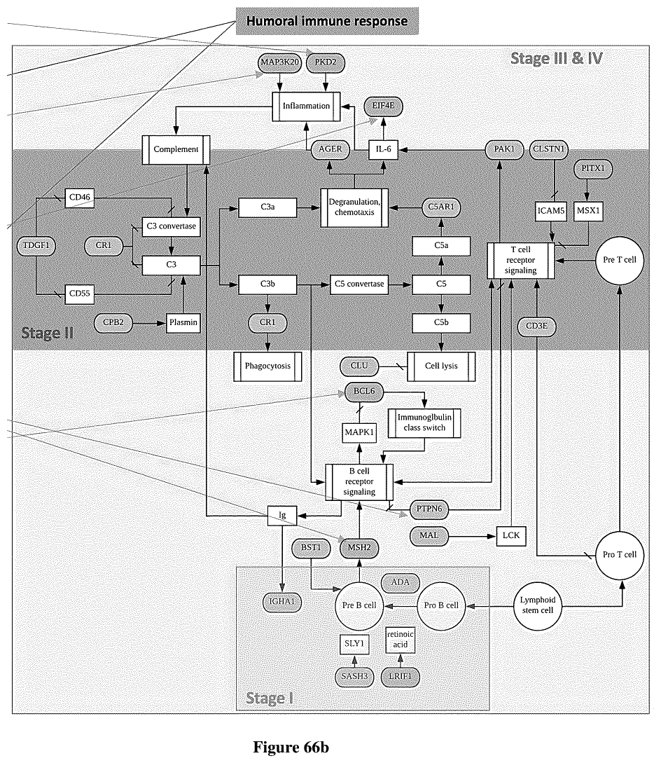

In one embodiment, this invention provides a method for diagnosing the staging of lung cancer. A sample is first obtained from a subject and gene expression (RNAs) was analyzed with whole-genome co-expressional changes using methods such as Next Generation Sequencing and Microarray technologies or any other methods known in the art. Our method was then used for conducting i) NPM1 Structural Gene Co-expression Analysis & Functional and Pathway Enrichment: NPM1 Correlated Genes in immune response specific to stage 1 (Table 6) & (FIGS. 59, 60, 61); ii) NPM1 Structural Gene Co-expression Analysis (Interconnected with Complement System): NPM1 Correlated Genes in immune response specific to stage II (Table 7) & (FIGS. 62, 63a, 63b), and NPM1 Correlated Genes in immune response specific to stage III & IV (Table 8) & (FIGS. 64a, 64b, 64c, 65)); iii) NPM1 Structural Gene Co-expression Analysis (Interconnected with Complement System): NPM1 Correlated Genes in phagocytosis with engulfment, mitotic cell cycle process and nucleosome assembly specific to late stages III & IV; Interactions of functional gene modules of all stages linking to carcinogenesis (FIGS. 66a, 66b). Table D shows the conditions used in this Example.

TABLE D
Conditions used in Example 5

| Parameter | | Condition |
|---|---|---|
| Genetic trait | | Cancer stage |
| State of interest | | Lung Adenocarcinoma cells under question |
| Reference state | | Lung Adenocarcinoma cells at known |
| | | cancer stage |
| Gene of interest | | NPM1-correlated genes appearing in FIGS. |
| | | 60, 61, 62, 63, 64, 65, 66a, 66b |
| Methods | Fiter gene | Data of cancer patients in stages 1-2 were |
| | expression data | obtained on online databases, with pre- |
| | | categorized data to show patients' stage |
| | Second gene | Data of cancer patients in stages 3-4 were |
| | expression data | obtained on online databases, with pre- |
| | | categorized data to show patients' stage |
| | First co- | NPM1 co-expression analysis with |
| | expression | MATLAB |
| | analysis | |
| | Second co- | NPM1 co-expression analysis with |
| | expression | MATLAB |
| | analysis | |
| | Differential | N/A |
| | expression | |
| | analysis | |
| | Connectivity | STRING |
| | functional | DAVID, ClueGO, QuickGO, KEGG |
| | enrichment or | |
| | pathway | |
| | enrichment | |

TABLE 6
Details of significant GO groups
for gene clusters in disease state at stage I.

| | | Term | | |
|---|---|---|---|---|
| | | p-Value | | |
| | | Corrected | | |
| | | with | | |
| | | Bonferroni | # of | Associated |
| GO ID | GO Term | step down | Genes | Genes Found |
| GO: | positive | 0.05 | 8.00 | [ADA, CD28, EXOSC3, |
| 0050871 | regulation | | | IGHA1, LRIF1, PGAP2, |
| | of B cell | | | PRDM6, SASH3] |
| | activation | | | |

TABLE 7
Details of significant GO groups for gene clusters in disease state at stage II.

| | | Term | | |
|---|---|---|---|---|
| | | p-Value | | |
| | | Corrected | | |
| | | with | | |
| | | Bonferroni | # of | |
| GO ID | GO Term | step down | Genes | Associated Genes Found |
| GO: | adaptive | 0.00 | 69.00 | [AGER, ALOX15, AMBP, ARG1, ATAD5, C1QC, CD1C, CD1D, CD3E, CD3G, CD40LG, |
| 0002250 | immune | | | CD79A, CD8B, CLC, CLEC6A, CLSTN1, CR1, CTLA4, CTSB, CTSH, DUSP10, EIF4E, |
| | response | | | FCGR2A, FOXJ1, FYN, FZD5, GAPT, GINS1, HFE, HNRNPC, HRAS, IFNA6, IL12A, IL27, |
| | | | | IL6, LILRB3, MEF2C, MRO, MYDGF, NCOR2, NECTIN2, NR2C2, NR4A3, ORAI1, PAK1, |
| | | | | PITX1, POU2F2, PPHLN1, PRKCZ, RAB27A, RIF1, RNF8, SEC14L3, SH2D1A, SKAP1, |
| | | | | SLA2, SLC22A3, SLC6A20, SPNS1, TDGF1, TDRD7, TFEB, TLR4, TNF, TNFRSF11A, |
| | | | | TNFRSF17, TNFSF4, TRAPPC13, TXK] |
| GO: | adaptive | 0.05 | 41.00 | [AGER, AMBP, ARG1, ATAD5, C1QC, CD1C, CD1D, CD40LG, CLC, CR1, CTSB, CTSH, |
| 0002460 | immune | | | FCGR2A, FOXJ1, FZD5, GAPT, HFE, HNRNPC, HRAS, IL12A, IL27, IL6, MEF2C, |
| | response | | | MYDGF, NECTIN2, NR2C2, PAK1, PITX1, POU2F2, PPHLN1, PRKCZ, RAB27A, RIF1, |
| | based on | | | RNF8, SLA2, TDGF1, TDRD7, TLR4, TNF, TNFSF4, TRAPPC13] |
| | somatic | | | |
| | re- | | | |
| | combination | | | |
| | of immune | | | |
| | receptors | | | |
| | built from | | | |
| | immuno- | | | |
| | globulin | | | |
| | superfamily | | | |
| | domains | | | |
| GO: | Complement | 0.04 | 13.00 | [C1QC, C5AR1, C5AR2, CFHR4, CPB2, CR1, FCN3, HNRNPC, PITX1, |
| 0006956 | activation | | | PPHLN1, TDGF1, VSIG4, VTN] |
| GO: | humoral | 0.02 | 10.00 | [AMBP, C1QC, CR1, FCGR2A, FOXJ1, HNRNPC, PITX1, PPHLN1, TDGF1, TNF] |
| 0002455 | immune | | | |
| | response | | | |
| | mediated | | | |
| | by | | | |
| | circulating | | | |
| | immuno- | | | |
| | globulin | | | |

TABLE 8
Details of significant GO groups for gene clusters in disease state at stage III and IV.

| | | Term | | Associated Genes Found |
|---|---|---|---|---|
| | | p-Value | | |
| | | Corrected | | |
| | | with | | |
| | | Bonferroni | # of | |
| GO ID | GO Term | step down | Genes | |
| GO: | adaptive | 0.00 | 111.00 | [ADAM17, AGER, APLF, ARG2, ARHGEF28, B2M, BCL6, BTF3, BTNL8, C1QBP, |
| 0002250 | immune | | | C8A, CAPG, CCL19, CCR6, CD19, CD46, CD79B, CD86, CD8B, CLSTN1, CLU, |
| | response | | | CXCL10, CXCL 13, DCLRE1C, DLG1, DUSP22, EIF2AK4, EIF4E, EOMES, EPHB6, |
| | | | | ERCC1, EXO1, EXOSC3, FCER2, FGB, FOXJ1, GNAO1, HELLS, HLX, HNRNPC, |
| | | | | HPRT1, IFNA17, IFNA5, IFNA6, IFNE, IGHA1, IL12B, IL18BP, IL23R, IL33, |
| | | | | ITPRID2, JAG1, JAK2, KLRK1, KRT32, LEF1, LILRA1, LILRB1, LIME1, LYN, |
| | | | | MAL, MAP3K20, MASP2, MICB, MRTFA, MSH2, NCOR2, NDFIP1, NLRP2, |
| | | | | OTUB1, PAK1, PARP2, PARP3, PKD2, PRKCD, PRKCQ, PRKD2, PROK1, PTPN6, |
| | | | | RAB27A, RAET1L, RIPK2, RNF8, SCYL1, SDCBP2, SEC14L2, SH2D1A, SLC25A4, |
| | | | | SMAD7, SPN, SYK, TADA1, TAP2, TARM1, TDGF1, TGFB1, THOC1, TINAGL1, |
| | | | | TNFRSF11A, TNFRSF14, TNFSF18, TRAPPC13, TRAPPC9, ULBP3, UNC93B1, |
| | | | | UNG, VTCN1, ZBTB1, ZBTB7B, ZC3H12A, ZNF395] |
| GO: | positive | 0.05 | 17.00 | [BAD, BCL6, BST1, EXOSC3, GALM, IGHA1, IL13, MAL, MRTFA, MSH2, |
| 0050871 | regulation | | | NFATC2, SLC39A10, SYK, TADA1, TGFB1, TRAPPC13, UNG] |
| | of B cell | | | |
| | activation | | | |
| GO: | B cell | 0.02 | 33.00 | [APLF, BCL6, C1QBP, C8A, CAPG, CCR6, CD19, CD46, CLU, CXCL10, ERCC1, |
| 0019724 | mediated | | | EXO1, EXOSC3, FCER2, FOXJ1, GNAO1, HNRNPC, IGHA1, MASP2, MSH2, |
| | immunity | | | NDFIP1, NLRP2, PARP2, PARP3, PRKCD, PTPN6, RNF8, SCYL1, TDGF1, TGFB1, |
| | | | | THOC1, TRAPPC13, UNG] |
| GO: | immuno- | 0.03 | 33.00 | [APLF, BCL6, C1QBP, C8A, CAPG, CCR6, CD19, CD46, CLU, CXCL10, ERCC1, |
| 0016064 | globulin | | | EXO1, EXOSC3, FCER2, FOXJ1, GNAO1, HNRNPC, IGHA1, MASP2, MSH2, |
| | mediated | | | NDFIP1, NLRP2, PARP2, PARP3, PRKCD, PTPN6, RNF8, SCYL1, TDGF1, TGFB1, |
| | immune | | | THOC1, TRAPPC13, UNG] |
| | response | | | |
| GO: | humoral | 0.00 | 15.00 | [C1QBP, C8A, CAPG, CD46, CLU, CXCL10, EXO1, FCER2, FOXJ1, GNAO1, |
| 0002455 | immune | | | HNRNPC, IGHA1, MASP2, PTPN6, TDGF1] |
| | response | | | |
| | mediated | | | |
| | by | | | |
| | circulating | | | |
| | immuno- | | | |
| | globulin | | | |
| GO | complement | 0.00 | 10.00 | [C1QBP, C8A, CAPG, CD46, CLU, CXCL10, HNRNPC, IGHA1, MASP2, TDGF1] |
| 0006958 | activation, | | | |
| | classical | | | |
| | pathway | | | |
| GO: | complement | 0.00 | 16.00 | [C1QBP, C3AR1, C8A, CAPG, CD19, CD46, CFHR4, CLU, CXCL10, CYP11B2, |
| 0006956 | activation | | | DBI, HNRNPC, IGHA1, MASP1, MASP2, TDGF1] |
| GO: | membrane | 0.01 | 15.00 | [ABCA1, ARF1P1, ARHGAP12, BIN2, CDK7, CHMP4A, ELMO1, GSN, GULP1, |
| 0010324 | invagination | | | IGHA1, MYH9, SLC52A1, SNX3, SRSF5, XKR4] |
| GO | plasma | 0.00 | 12.00 | [ABCA1, ARF1P1, ARHGAP12, BIN2, CDK7, ELMO1, GSN, GULP1, IGHA1, |
| 0099024 | membrane | | | MYH9, SLC52A1, XKR4] |
| | invagination | | | |
| GO: | phagocytosis, | 0.00 | 10.00 | [ABCA1, ARHGAP12, BIN2, ELMO1, GSN, GULPI, IGHA1, MYH9, SLC52A1, |
| 0006911 | engulfment | | | XKR4] |
| GO: | mitotic cell | 0.02 | 305.00 | [ABRAXAS2, ACP2, ACTR1B, ADAM17, AGFG1, AKAP9, ALMS1, ANAPC10, |
| 1903047 | cycle | | | ANAPC4, ANKLE2, ANKRD26, ANLN, APEX1, AQP6, ARFIP1, ARL3, ARPP19, |
| | process | | | ATF2, AURKA, AZIN1, BAX, BCCIP, BCL6, BMP4, BOD1, BORA, BRD4, BRSK1, |
| | | | | BRSK2, BTC, BUBI, BUB3, CALM2, CALM3, CAPG, CARM1, CAV2, CBX8, |
| | | | | CCDC88A, CCN1, CCNA1, CCND1, CCNE2, CCNG2, CCNI2, CCNJ, CCNL1, |
| | | | | CCNY, CCP110, CCSAP, CDC23, CDC27, CDC34, CDC42EP2, CDC7, CDK1, |
| | | | | CDK5RAP2, CDK6, CDK7, CDKN2A, CDKN2B, CDKN3, CDS1, CENPE, CENPJ, |
| | | | | CEP135, CEP55, CEP70, CFL1, CHMP1B, CHMP4A, CHMP6, CHMP7, CIB1, CIT, |
| | | | | CKAP2, CKAPS, CNOT1, CNOT4, CNOT6, CRLF3, CTDSP1, CTDSPL, CUL1, |
| | | | | CUL2, CUL3, CUL4B, CULS, CXXC1, DAB2IP, DBF4, DCTN1, DCTN2, DDB1, |
| | | | | DDX19B, DDX3X, DIS3L2, DLC1, DLGI, DLGAP5, DNA2, DNM2, DONSON, |
| | | | | DSCC1, DTL, DUSP12, DYNC1LI1, DYNLT1, DYRK3, E2F7, ECD, ECT2, EIF4E, |
| | | | | ELP4, EME2, ESRRB, FAM107A, FBXL12, FEZ1, FSD1L, GPAM, HASPIN, HAUS1, |
| | | | | HAUS4, HAUS6, HMMR, HUS1B, IK, IL1A, KANSL1, KAT14, KCNH5, KHDRBS1, |
| | | | | KIF14, KIF18A, KIF20A, KIF20B, KLF4, KLHL22, KMT2E, KNSTRN, KNTC1, |
| | | | | LATS2, LIG1, LRP5, MAGI2, MAP3K20, MAP4, MAP9, MCM10, MEPCE, MIS12, |
| | | | | MNAT1, MRE11, MSH2, MUS81, NAA20, NAA30, NCAPG, NCAPH, NEDD1, |
| | | | | NEK2, NEK6, NEK8, NEUROG1, NINL, NLRP2, NOP53, NPAT, NSFL1C, NUF2, |
| | | | | OFD1, OLAH, ORC2, ORCS, OVOL2, PAK1IP1, PAMR1, PARP2, PARP3, PBK, |
| | | | | PCM1, PCNA, PCSK1, PDCD6IP, PDSSA, PDSSB, PHB2, PHF 13, PIAS1, PIDDI, |
| | | | | PKD1, PKD2, PLAGL1, PLK4, PLP2, PMEL, POGZ, POLA1, POLE2, PPAT, |
| | | | | PPP1R12A, PPP1R3D, PPP2R2A, PRDX5, PRIM1, PRKAR2B, PRKCA, PRKD2, |
| | | | | PRMT2, PRMT5, PROC, PRSS27, PSMA1, PSMA3, PSMA4, PSMA6, PSMB4, |
| | | | | PSMC2, PSMD10, PSMD12, PSMD13, PSMD4, PSMD6, PSMD7, PSME1, PSME2, |
| | | | | PSMG2, PTPN6, PTTG1, PTTG3P, RAD17, RAD9A, RASAI, RBL2, RECQL5, |
| | | | | REEP4, REEP5, RHOB, RHOC, RINT1, RIOK2, RPA1, RPA3, RPA4, RPAIN, |
| | | | | RPS6KB1, RRS1, RTEL1, SBDS, SCP2, SDCCAG8, I1L, SEMI, SETMAR, SFN, |
| | | | | SGCG, SGO1, SIRT7, SKP1, SLC11A2, SLC25A2, SLC4A11, SLF2, SMC2, SMC3, |
| | | | | SOX4, SPAG5, SPTBN1, STK33, STOX1, SYCP1, TAF2, TAOK3, TBCE, TENT4A, |
| | | | | TERT, TFDP2, TGFB1, TGFBR1, TIPIN, TMEM14B, TMPRSS4, TOP2A, TOPBP1, |
| | | | | TPR, TYMS, UBE2D1, UBE2E2, USP16, USP17L2, USP21, USP37, USP47, VPS4A, |
| | | | | VPS4B, WDR11, WEE1, WRN, ZFP36L2, ZMPSTE24, ZNF207, ZNF22, ZNF365, |
| | | | | ZWINT] |
| GO: | nucleosome | 0.02 | 20.00 | [ARID2, ASF1A, CABIN1, CCNL1, CDAN1, CENPH, CENPN, CENPO, CENPQ, |
| 0034728 | organization | | | CHD6, CTDSPL, H2AX, KALRN, KAT6B, MIS18BP1, NAP1L3, RNF8, SMARCE1, |
| | | | | SUPT16H, TSPYL1] |
| GO: | nucleosome | 0.01 | 13.00 | [ASF1A, CABIN1, CCNL1, CDAN1, CENPH, CENPN, CENPO, CENPQ, H2AX, |
| 0006334 | assembly | | | KAT6B, MIS18BP1, NAPIL3, TSPYL1] |

### Example 6

### Diagnosing Tumorigenesis of Small Cell Lung Cancer and Platinum Drug Resistance

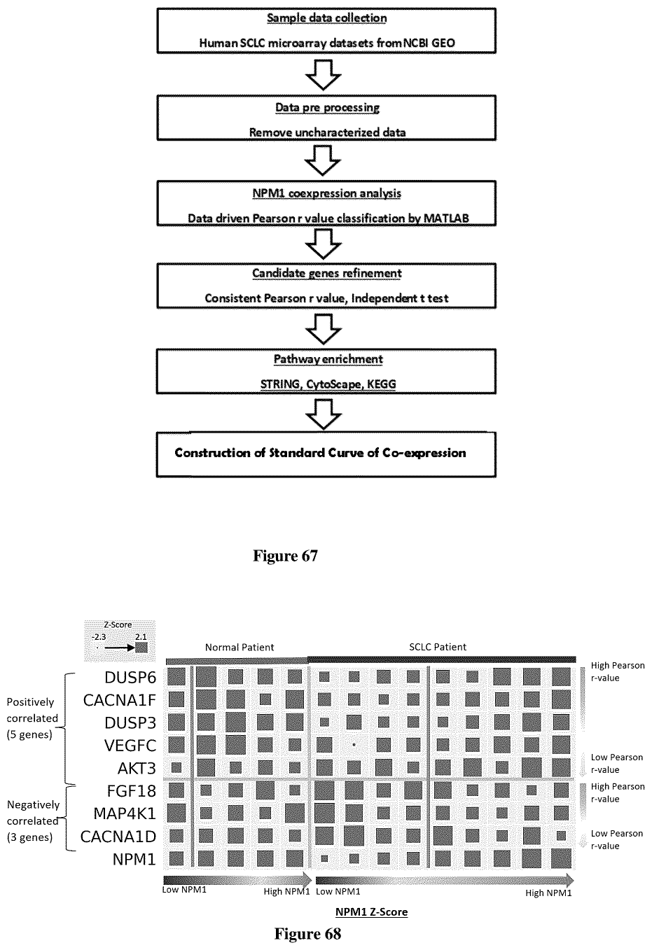

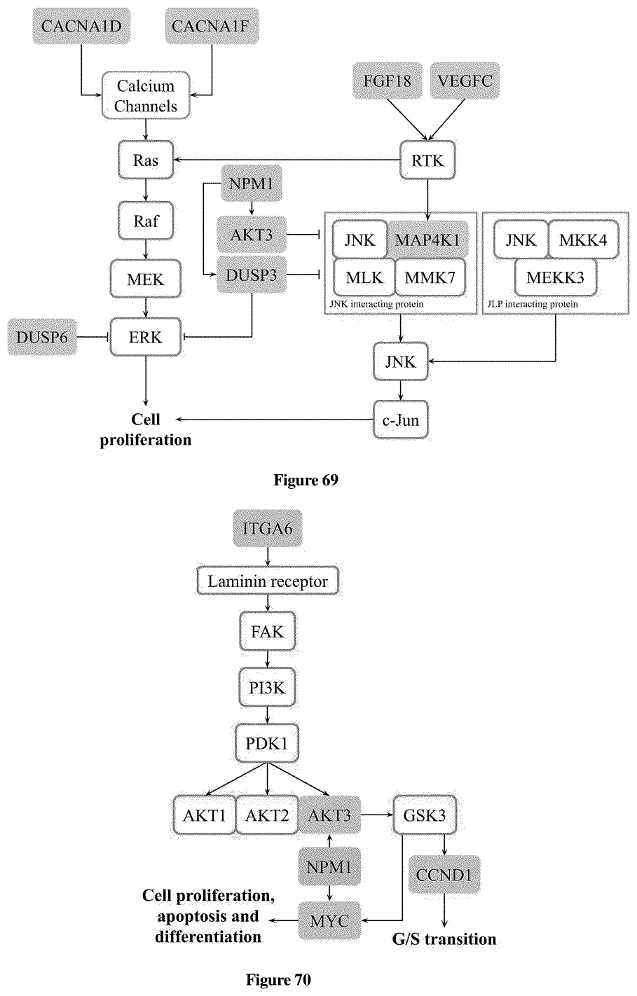

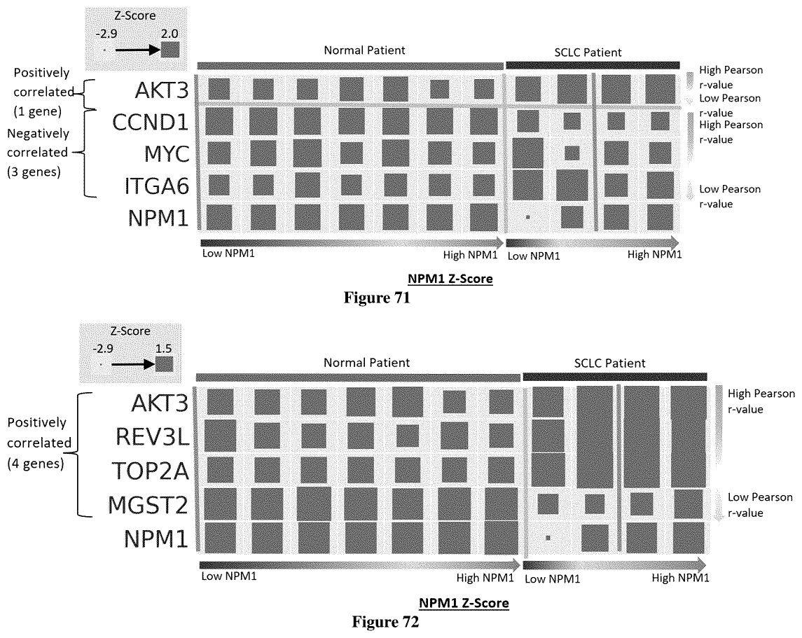

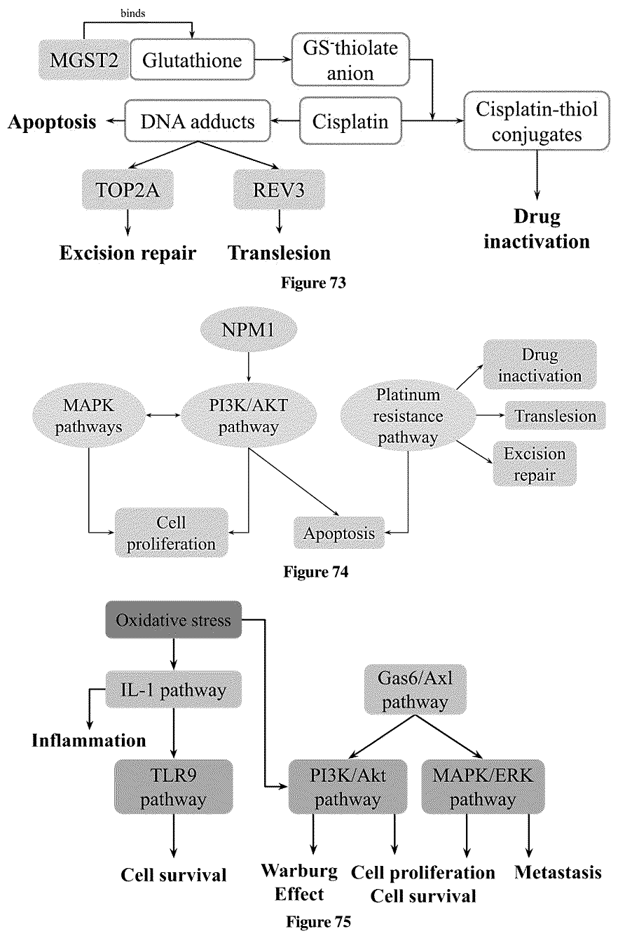

In one embodiment, this invention provides a method for diagnosing the tumorigenesis of small cell lung cancer (SCLC) and platinum drug resistance (FIG. 67). A sample is first obtained from a subject and gene expression (RNAs) was analyzed with whole-genome co-expressional changes using methods such as Next Generation Sequencing and Microarray technologies or any other methods known in the art. Our method was then used for conducting NPM1 Structural Gene Co-expression Analysis & Functional and Pathway Enrichment: NPM1 Correlated Genes (DUSP6, CACNA1D, DUSP3, VEGFC, AKT3, FGF18, MAP4K1, CACNA1F) in MAPK signaling pathways (FIGS. 68, 69), NPM1 Correlated Genes (ITGA6, AKT3, CCND1, MYC) in PI3K/AKT pathways (FIGS. 70, 71), and NPM1 Correlated Genes (AKT3, REV3L, TOP2A, MGST2) in platinum drug resistance (FIGS. 72, 73, 74). Table E1 and Table E2 show the conditions used in this Example.

TABLE E1
Conditions used in Example 6

| Parameter | | Condition |
|---|---|---|
| Genetic trait | | Tumorigenesis |
| State of interest | | Alveolar cells under question |
| Reference state | | Small Cell Lung Cancer Cells |
| Gene of interest | | NPM1-correlated genes appearing |
| | | in FIGS. 68, 69, 70, 71 |
| Methods | First gene | Data of SCLC patients were obtained on |
| | expression | online databases, with pre-categorized |
| | data | data to show malignancy of the tissue |
| | Second gene | Data of normal lung tissue were obtained |
| | expression | on online databases, with pre-categorized |
| | data | data to show malignancy of the tissue |
| | First co- | NPM1 co-expression analysis with |
| | expression | MATLAB |
| | analysis | |
| | Second co- | NPM1 co-expression analysis with |
| | expression | MATLAB |
| | analysis | |
| | Differential | N/A |
| | expression | |
| | analysis | |
| | Connectivity | STRING |
| | functional | ClueGO, KEGG, Reactome |
| | enrichment or | |
| | pathway | |
| | enrichment | |

TABLE E2
Conditions used in Example 6

| Parameter | | Condition |
|---|---|---|
| Genetic trait | | Platinum drug resistance |
| State of interest | | Small Cell Lung Cancer Cells |
| | | under question |
| Reference state | | Small Cell Lung Cancer Cells |
| | | with Platinum drug resistance |
| Gene of interest | | NPM1-correlated genes |
| | | appearing in FIGS. 72, 73, 74 |
| Methods | First gene expression data | SCLC Patients |
| | Second gene expression data | Normal lung tissue |
| | First co-expression analysis | NPM1 co-expression analysis |
| | | with MATLAB |
| | Second co-expression analysis | NPM1 co-expression analysis |
| | | with MATLAB |
| | Differential expression analysis | N/A |
| | Connectivity | STRING |
| | functional enrichment or | ClueGO, KEGG, Reactome |
| | pathway enrichment | |

### Example 7

### Diagnosing Tumorigenesis of Hepatocellular Carcinoma

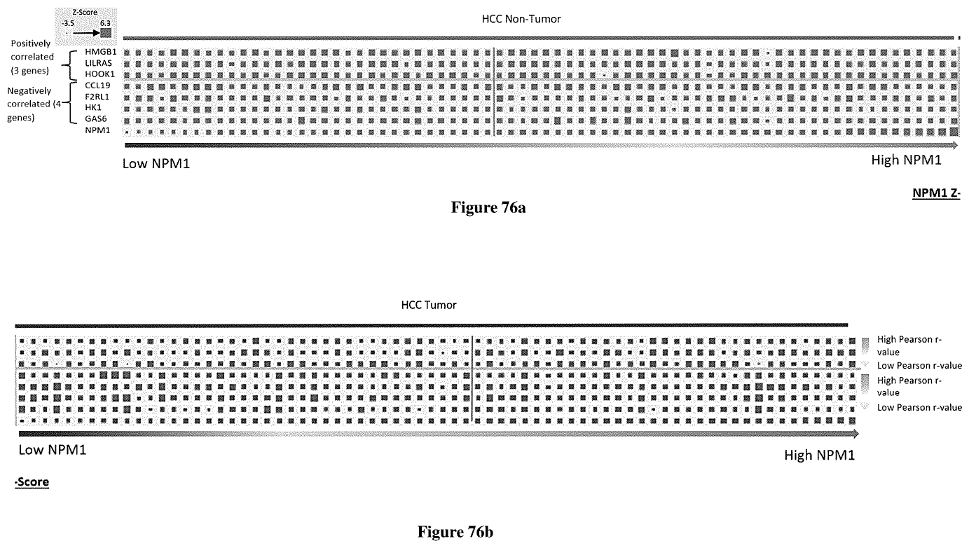

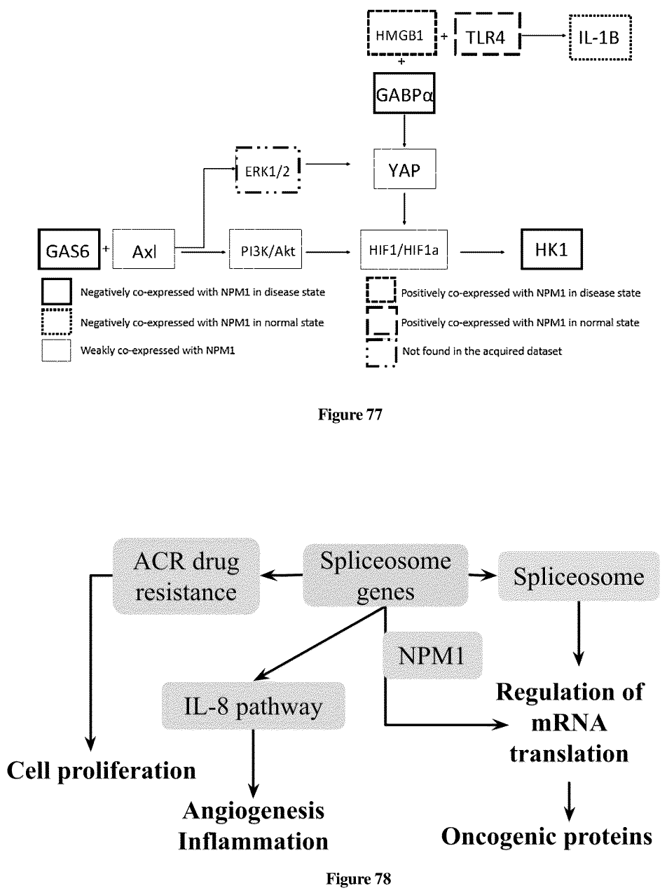

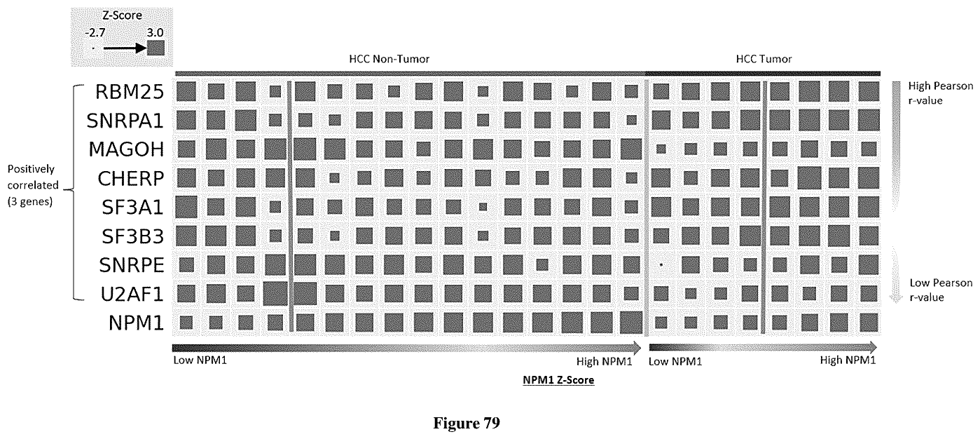

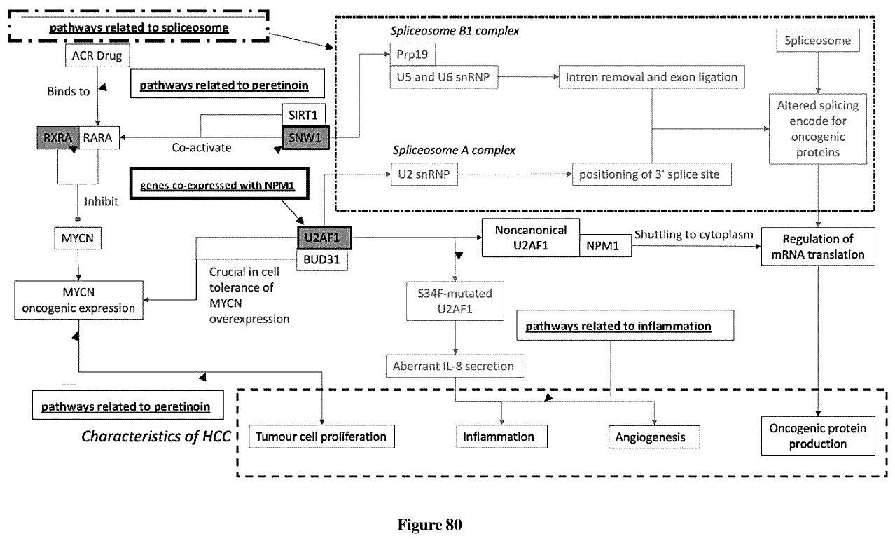

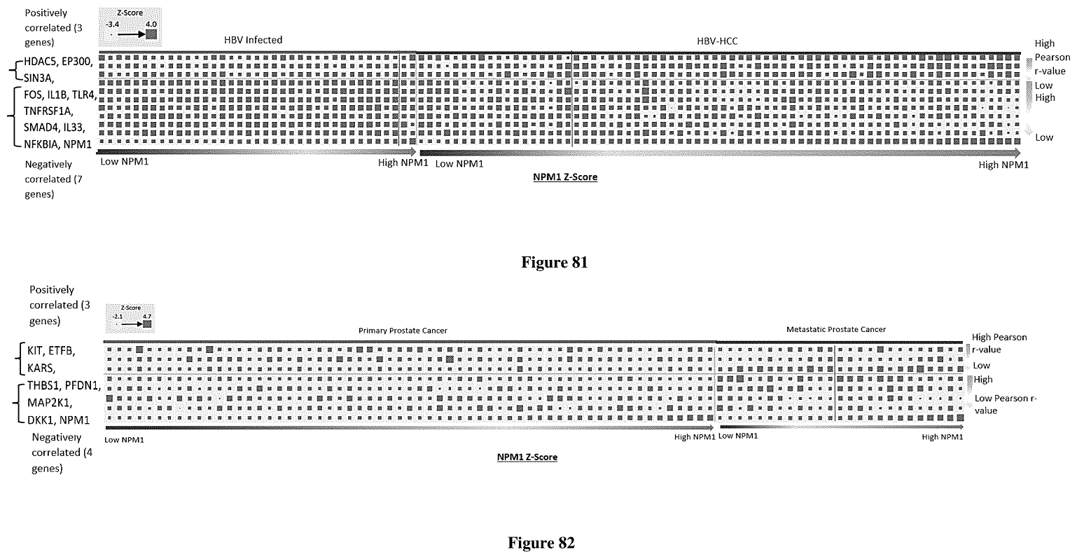

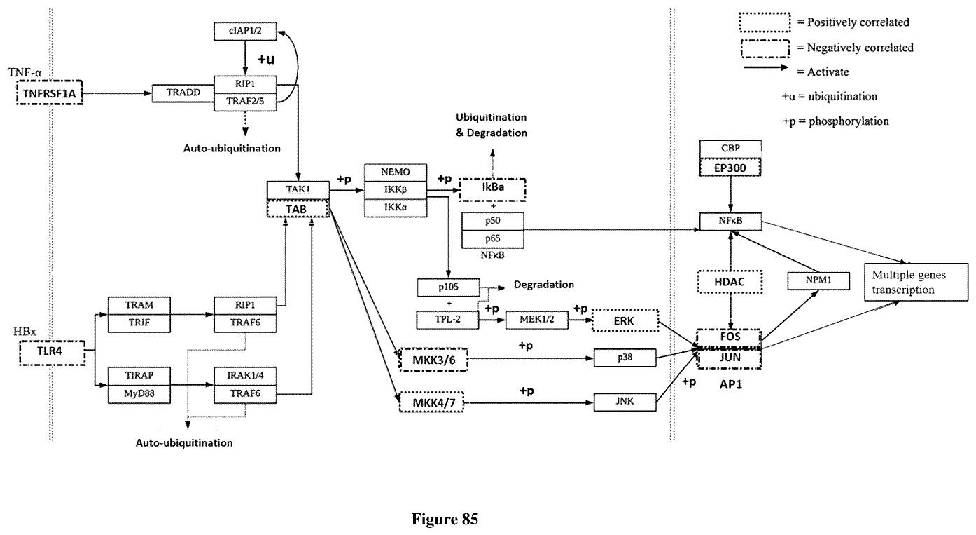

In one embodiment, this invention provides a method for diagnosing the tumorigenesis of Hepatocellular Carcinoma (HCC). A sample is first obtained from a subject and gene expression (RNAs) was analyzed with whole-genome co-expressional changes using methods such as Next Generation Sequencing and Microarray technologies or any other methods known in the art. Our method was then used for conducting NPM1 Structural Gene Co-expression Analysis & Functional and Pathway Enrichment, NPM1 Correlated Genes in (HMGB1, LILRAS, HOOK1, CCL19, F2RL1, HK1, GAS6) Interleukin-1 pathway (FIGS. 75, 76a, 76b, 77) & (Tables 9, 10), NPM1 Correlated Genes in (RBM25, SNRPA1, MAGOH, CHERP, SF3A1, SFSB3, SNRPE, SNW1, U2AF1) Spliceosome gene regulation (FIGS. 78, 79, 80), NPM1 Correlated Genes (HDAC5, EP300, SIN3A, FOS, IL1B, TLR4, TNFRSF, SMAD4, IL33, NFKBIA) in the NFκB signaling network in HBV-associated HCC (FIGS. 81, 84, 85) & (Table 11). Tables F1, F2, F3 show the conditions used in this Example.

TABLE F1
Conditions used in Example 7

| Parameter | | Condition |
|---|---|---|
| Genetic trait | | Tumorigenesis |
| State of interest | | Liver cells under question |
| Reference state | | Hepatocellular Carcinoma Cells |
| Gene of interest | | NPM1-correlated genes appearing in |
| | | FIGS. 75, 76a, 76b, 77, 78, 79, |
| | | 80, 81, 84, 85 |
| Methods | First gene | Data of HCC tumor cells were obtained on |
| | expression data | online databases, with pre-categorized data |
| | | to show malignancy of the tissue |
| | Second gene | Data of non-tumor surrounding liver |
| | expression data | tissues were obtained on online databases, |
| | | with pre-categorized data to show |
| | | malignancy of the tissue |
| | First | NPM1 co-expression analysis with |
| | co-expression | MATLAB |
| | analysis | |
| | Second | NPM1 co-expression analysis with |
| | co-expression | MATLAB |
| | analysis | |
| | Differential | N/A |
| | expression | |
| | analysis | |
| | Connectivity | STRING |
| | functional | ClueGO, KEGG, Reactome, Biocarta, |
| | enrichment or | DAVID |
| | pathway | |
| | enrichment | |

TABLE F2
Conditions used in Example 7

| Parameter | | Condition |
|---|---|---|
| Genetic trait | | Tumorigenesis |
| State of interest | | Liver cells under question |
| Reference state | | Hepatocellular Carcinoma Cells |
| Gene of interest | | NPM1-correlated genes appearing in |
| | | FIGS. 75, 76a, 76b, 77, 78, |
| | | 79, 80, 81, 84,85 |
| Methods | First gene | Data of HBV-infected Hepatocellular |
| | expression data | Carcinoma Cells were obtained on online |
| | | databases, with pre-categorized data |
| | | to show presence/absence of HCC |
| | | in the HBV-infected cells |
| | Second gene | Data of HBV-infected normal cells |
| | expression data | were obtained on online databases, |
| | | with pre-categorized data to show |
| | | presence/absence |
| | | of HCC in the HBV-infected cells |
| | First | NPM1 co-expression analysis with |
| | co-expression | MATLAB |
| | analysis | |
| | Second | NPM1 co-expression analysis with |
| | co-expression | MATLAB |
| | analysis | |
| | Differential | N/A |
| | expression | |
| | analysis | |
| | Connectivity | STRING |
| | functional | ClueGO (Gene Ontology), KEGG, |
| | enrichment or | QuickGO, Reactome, |
| | pathway | BioCarta, DAVID |
| | enrichment | |

TABLE F3
Conditions used in Example 7

| Parameter | | Condition |
|---|---|---|
| Genetic trait | | Tumorigenesis |
| State of interest | | Liver cells under question |
| Reference state | | Hepatocellular Carcinoma Cells |
| Gene of interest | | NPM1-correlated genes appearing in |
| | | FIGS. 75, 76a, 76b, 77, 78, |
| | | 79, 80, 81, 84, 85 |
| Methods | First gene | Data of HCC relapse patient HCC tissue |
| | expression data | were obtained on online databases, |
| | | with pre-categorized data to show |
| | | absence/presence of relapse |
| | Second gene | Data of HCC non-relapse patient |
| | expression data | HCC tissue were obtained |
| | | on online databases, |
| | | with pre-categorized data to show |
| | | absence/presence of relapse |
| | First co-expression | NPM1 co-expression analysis with |
| | analysis | MATLAB |
| | Second co-expression | NPM1 co-expression analysis with |
| | analysis | MATLAB |
| | Differential expression | N/A |
| | analysis | |
| | Connectivity | STRING |
| | functional enrichment | ClueGO (Gene Ontology), KEGG, |
| | or | QuickGO, DAVID |
| | pathway enrichment | |

TABLE 9
Result of ‘disease’ doublets after functional annotation analysis (adaptive immune response related)

| GOI | GO Term | Associated Genes Found |
|---|---|---|
| GO: 0002□50 | Adaptive i□m□ne | AIRE, BTN3A1, CBLIF, CCL19, CD1E, CD5, CD6, CD7, CTLA4, FCAMR, |
| | response | FH, GPR183, GTF2F2, HMGB1, HMHB1, IFNA1, IFNA14, IL2, IL20RB, |
| | | IL33, INPP5D, KDELR1, KRT32, MALT1, NEDD4, NFKBIZ, NLRP2, |
| | | PRDM1, PRKCQ, PYCARD, RC3H1, SLAMF1, SLC6A20, |
| | | SWAP70, TDRD7, TNFRSF17, TNFSF13B, TNFSF18, TXK, ZNF683 |
| GO: 0002325 | Natural killer | PGLYRP1, PGLYRP4, ZNF683 |
| | cell differentiation | |
| | involved in | |
| | immune response | |
| GO: 0002377 | Immunoglobulin | CARD11, GALNT2, IL2, IL33, NLRP2, POLB, SWAP70, TCF3, |
| | production | TDRD7, TMBIM6, TNFSF13B |
| GO: 0002443 | Leukocyte mediated | AIRE, ALAD, ALOX5, ARHGAP9, ATP11B, C1orf35, CBLIF, CD1E, CD44, |
| | immunity | CEBPG, CHST4, CRISPLD2, CTSZ, CYFIP1, DEFA4, DOK3, EPX, F2RL1, |
| | | FES, FGL2, FH, GDI2, GHDC, GM2A, GTF2F2, HMGB1, HSP90AA1, |
| | | HSPA1A, IL2, IL20RB, INPPSD, ITGB2, ITM2C, KDELR1, KIR3DL1, KLK8, |
| | | KRT32, MALT1, MILR1, MMP9, MTCH1, NAT8, NLRP2, PGLYRP1, PKP1, |
| | | PPIP5K1, PSMA2, PYCARD, RAB6A, RAC1, RNASE1, ROCK1, S100A7, |
| | | SCAMP1, SERPINB10, SLC18A1, SLC2A3, SWAP70, TDRD7, TLR2, |
| | | TRAPPC1, TRIT1, TSPAN14, UBR4, WDR1 |
| GO: 0002460 | Adaptive immune | AIRE, CBLIF, CCL19, CD1E, CD5, GTF2F2, HMGB1, IL2, IL20RB, |
| | response based | IL33, INPP5D, KDELR1, KRT32, MALT1, NFKBIZ, |
| | on somatic | NLRP2, PRKCQ, RC3H1, SWAP70, TDRD7, TNFSF13B |
| | recombination of | |
| | immune receptors | |
| | built from | |
| | immunoglobulin | |
| | superfamily domains | |
| GO: 0032823 | Regulation of | GAS6, PGLYRP1, PGLYRP4, PRDM1, ZNF683 |
| | natural killer cell | |
| | differentiation | |
| GO: 0032826 | Regulation of | PGLYRP1, PGLYRP4, ZNF683 |
| | natural killer | |
| | cell differentiation | |
| | involved in | |
| | immune response | |
| GO: 1903039 | Positive regulation | AIF1, BTN2A2, BTNL2, CARD11, CCL19, CD276, CD44, CD5, CD6, |
| | of leukocyte | CD83, CTLA4, DPP4, EFNB1, EPX, GATD3A, GLI2, HMGB1, IHH, |
| | cell-cell adhesion | IL2, KLK8, MALT1, NFKBIZ, PAK3, PRKCQ, PYCARD, RAC1, RARA, |
| | | RASAL3, RELA, RNASE1, SIRPG, TDRD7, TNFSF13B, UMOD |
| GO: 0042110 | T cell activation | AIF1, BTN2A2, BTNL2, CARD11, CCL19, CD276, CD44, CD5, CD6, CD83, |
| | | CTLA4, DPP4, EFNB1, EPX, GATD3A, GLI2, HMGB1, IHH, |
| | | IL2, KLK8, MALT1, NFKBIZ, PAK3, PRKCQ, PYCARD, RAC1, RARA, |
| | | RASAL3, RELA, RNASE1, SIRPG, TDRD7, TNFSF13B, UMOD |
| GO: 2000316 | Regulation of | IL2, MALT1, NFKBIZ, PRKCQ, RC3H1 |
| | T-helper 17 type | |
| | immune response | |
| GO: 0050853 | B cell receptor | CD38, CSE1L, CTLA4, ELOF1, FOXP1, PLEKHAI, STAP1 |
| | signaling pathway | |
| GO: 0050870 | positive regulation | AIF1, BTN2A2, BTNL2, CARD11, CCL19, CD276, CD5, CD6, CD83, |
| | of T cell activation | CTLA4, DPP4, EFNB1, EPX, GATD3A, GLI2, HMGB1, |
| | | IHH, IL2, KLK8, MALT1, NFKBIZ, PAK3, PRKCQ, PYCARD, RAC1, |
| | | RARA, RASAL3, RNASE1, SIRPG, TDRD7, TNFSF13B, UMOD |
| GO: 0046637 | regulation of alpha- | CCL19, CD83, HMGB1, IHH, IL2, KLK8, MALTI, NFKBIZ, PRDM1, |
| | beta T cell | RARA, RC3H1, ZNF683 |
| | differentiation | |

TABLE 10
Result of ‘disease’ doublets after functional annotation analysis (cytokine secretion related)

| GO ID | GO Term | Associated Genes Found |
|---|---|---|
| GO: 0050663 | Cytokine secretion | AGT, AGXT, AIF1, ASPA, BTN2A2, BTN3A1, BTNL2, C1D, CARD11, CASP1, CAVIN3, |
| | | CCL19, CD5, CLEC9A, DHX9, F2RL1, FFAR2, FOXP1, GAS6, GTF2F2, HK1, HMGB1, |
| | | HMGB4, HMHB1, HOOK1, IL33, INS, IRF3, KLK8, LILRA5, NANOS2, NLRP2, |
| | | PLPPR4, PPIP5K1, PYCARD, RGCC, TLR2, USP50 |
| GO: 0050701 | Interleukin-1 secretion | CASP1, CCL19, F2RL1, FOXP1, GAS6, HK1, HMGB1, HMGB4, HOOK1, |
| | | LILRA5, NLRP2, PYCARD, USP50 |
| GO: 0050702 | Interleukin-1 beta secretion | CASP1, CCL19, F2RL1, FOXP1, GAS6, HK1, HMGB1, HMGB4, HOOK1, LILRA5, |
| | | NLRP2, PYCARD, USP50 |
| GO: 0050704 | Regulation of interleukin-1 | CASP1, CCL19, FOXP1, GAS6, HK1, HMGB1, HMGB4, HOOK1, LILRA5, |
| | secretion | NLRP2, PYCARD, USP50 |
| GO: 0050706 | Regulation of interleukin- | CASP1, CCL19, FOXP1, HK1, HMGB1, HMGB4, HOOK1, LILRA5, |
| | 1 beta secretion | NLRP2, PYCARD, USP50 |
| GO: 0050715 | Positive regulation of | AIF1, BTNL2, C1D, CASP1, CAVIN3, CCL19, CD5, CLEC9A, DHX9, F2RL1, FFAR2, |
| | cytokine secretion | HK1, HMGB1, HMGB4, HMHB1, HOOK1, IL33, INS, IRF3, LILRA5, |
| | | NLRP2, PPIP5K1, PYCARD, RGCC, TLR2, USP50 |
| GO: 0050716 | Positive regulation of | CASP1, CCL19, HK1, HMGB1, HMGB4, HOOK1, LILRA5, NLRP2, PYCARD, USP50 |
| | interleukin-1 secretion | |
| GO: 0050718 | Positive regulation of | CASP1, CCL19, HK1, HMGB1, HMGB4, HOOK1, LILRA5, NLRP2, PYCARD, USP50 |
| | interleukin-1 beta secretion | |
| GO: 0032627 | Interleukin-23 production | CSF2, RAC1, RNASE1 |
| GO: 0032661 | Regulation of interleukin-18 | DHX9, TLR2, USP50 |
| | production | |
| GO: 0032667 | Regulation of interleukin-23 | CSF2, RAC1, RNASE1 |
| | production | |
| GO: 0032673 | Regulation of interleukin-4 | CD83, EPX, IL20RB, IL33, PRKCQ, RARA, TDRD7 |
| | production | |
| GO: 0032689 | Negative regulation of | CD5, GAS6, HMGB1, IL20RB, IL33, INHBA, PGLYRP1, PGLYRP4, RARA |
| | interferon-gamma production | |
| GO: 0032728 | Positive regulation of | DHX9, IRF3, MRPL13, POLR3C, PPIPSK1, RIOK3, TLR2 |
| | interferon-beta production | |
| GO: 0032731 | Positive regulation of | CASP1, CCL19, EGR1, HK1, HMGB1, HMGB4, HOOK1, |
| | interleukin-1 beta production | LILRA5, NLRP2, PYCARD, USP50 |
| GO: 0032741 | Positive regulation of | DHX9, TLR2, USP50 |
| | interleukin-18 production | |
| GO: 0032753 | Positive regulation of | EPX, IL20RB, IL33, PRKCQ, RARA, TDRD7 |
| | interleukin-4 production | |
| GO: 0072641 | Type I interferon secretion | DHX9, HMGB1, HMGB4, PPIP5K1 |
| GO: 0072642 | Interferon-alpha secretion | DHX9, HMGB1, HMGB4, PPIP5K1 |
| GO: 1902739 | Regulation of interferon- | DHX9, HMGB1, HMGB4, PPIP5K1 |
| | alpha secretion | |
| GO: 1902741 | Positive regulation of | DHX9, HMGB1, HMGB4, PPIP5K1 |
| | interferon-alpha secretion | |
| GO: 0090195 | Chemokine secretion | AIF1, C1D, CD5, F2RL1, FOXP1, IL33, PYCARD |
| GO: 0090196 | Regulation of chemokine | AIF1, C1D, CD5, F2RL1, IL33, PYCARD |
| | secretion | |

TABLE 11
Gene ontology (GO) analysis of HBV induced HCC specific co-expressed genes. 29 enriched
biological pathways were identified by GO analysis when p-value was gated at <0.05. The 29
enriched biological pathways were organized into 3 GO pathway groups. Biological pathways
were first sorted by GO pathway groups and followed by p-value according to descending
order in the following table.

| GO Group | GO ID | Term/gene function | Count | P-value |
|---|---|---|---|---|
| Group 0 | GO: 0035239 | tube morphogenesis | 117.00 | 0.03 |
| Circulatory | GO: 0048514 | blood vessel morphogenesis | 91.00 | 0.01 |
| system | GO: 0072359 | circulatory system development | 148.00 | 0.01 |
| | GO: 0001568 | blood vessel development | 102.00 | 0.00 |
| | GO: 0035295 | tube development | 143.00 | 0.00 |
| | GO: 0001944 | vasculature development | 106.00 | 0.00 |
| | GO: 0072358 | cardiovascular system development | 109.00 | 0.00 |
| Group 1 | GO: 0031323 | regulation of cellular metabolic process | 615.00 | 0.05 |
| Regulation | GO: 0019222 | regulation of metabolic process | 652.00 | 0.04 |
| of | GO: 0048522 | positive regulation of cellular process | 549.00 | 0.02 |
| metabolic | GO: 0060255 | regulation of macromolecule metabolic process | 612.00 | 0.02 |
| process | GO: 0051171 | regulation of nitrogen compound metabolic process | 592.00 | 0.02 |
| | GO: 0051173 | positive regulation of nitrogen compound metabolic process | 359.00 | 0.01 |
| | GO: 0031325 | positive regulation of cellular metabolic process | 375.00 | 0.00 |
| | GO: 0080090 | regulation of primary metabolic process | 614.00 | 0.00 |
| | GO: 0048518 | positive regulation of biological process | 626.00 | 0.00 |
| | GO: 0010604 | positive regulation of macromolecule metabolic process | 380.00 | 0.00 |
| | GO: 0009893 | positive regulation of metabolic process | 408.00 | 0.00 |
| Group 2 | GO: 0006366 | transcription by RNA polymerase II | 318.00 | 0.04 |
| Metabolic | GO: 0044260 | cellular macromolecule metabolic process | 778.00 | 0.02 |
| process | GO: 0016070 | RNA metabolic process | 491.00 | 0.01 |
| | GO: 0010467 | gene expression | 556.00 | 0.00 |
| Group 2 | GO: 0006139 | nucleobase-containing compound metabolic process | 599.00 | 0.00 |
| Metabolic | GO: 0034641 | cellular nitrogen compound metabolic process | 657.00 | 0.00 |
| process | GO: 0006725 | cellular aromatic compound metabolic process | 619.00 | 0.00 |
| | GO: 0046483 | heterocycle metabolic process | 617.00 | 0.00 |
| | GO: 0090304 | nucleic acid metabolic process | 547.00 | 0.00 |
| | GO: 0043170 | macromolecule metabolic process | 868.00 | 0.00 |
| | GO: 1901360 | organic cyclic compound metabolic process | 642.00 | 0.00 |

### Example 8

### Diagnosing Prostate Cancer in Metastasis Stage

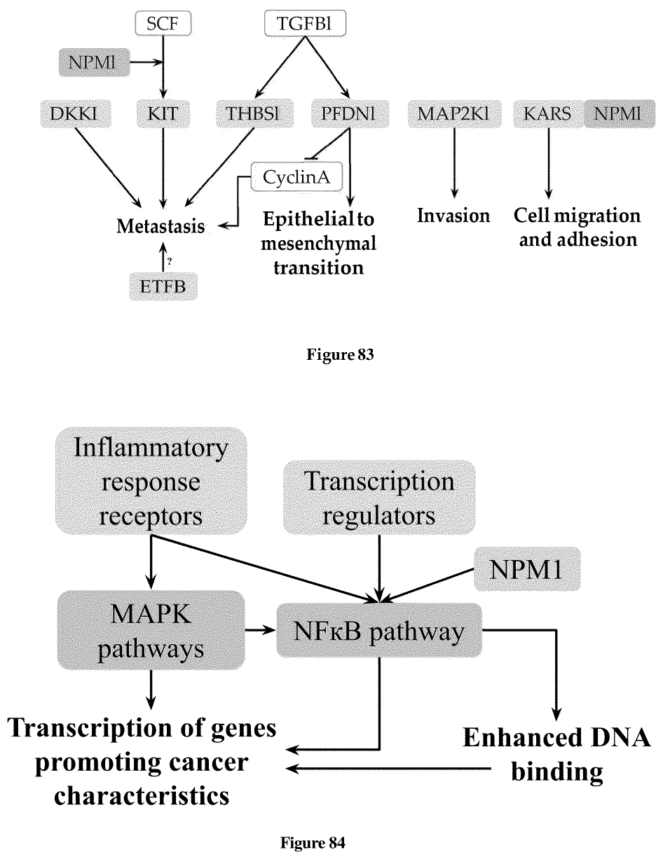

In one embodiment, this invention provides a method for diagnosing the prostate cancer in Metastasis Stage. A sample is first obtained from a subject and gene expression (RNAs) was analyzed with whole-genome co-expressional changes using methods such as Next Generation Sequencing and Microarray technologies or any other methods known in the art. Support Vector Machine was then used for conducting NPM1 Structural Gene Co-expression Analysis & Functional and Pathway Enrichment: NPM1 Correlated Genes (KIT, ETFB, KARS, THBS1, PFDN1, MAP2K1, DKK1) in Metastasis Stage (FIGS. 82, 83). Table G shows the conditions used in this Example.

TABLE G
Conditions used in Example 8

| Parameter | | Condition |
|---|---|---|
| Genetic trait | | Metastasis stage |
| State of interest | | Prostate cancer cells under question |
| Reference state | | Prostate cancer cells at a known |
| | | metastasis stage |
| Gene of interest | | NPM1-correlated genes appearing |
| | | in FIGS. 82, 83 |
| Methods | First gene | Data of metastatic prostate cancer tissue |
| | expression | were obtained on online databases, with pre- |
| | data | categorized data to show state of metastasis |
| | Second gene | Data of primary metastatic prostate cancer |
| | expression | tissue were obtained on online databases, |
| | data | with pre-categorized data to show state of |
| | | metastasis |
| | First | NPM1 co-expression analysis with |
| | co-expression | MATLAB |
| | analysis | |
| | Second | NPM1 co-expression analysis with |
| | co-expression | MATLAB |
| | analysis | |
| | Differential | N/A |
| | expression | |
| | analysis | |
| | Connectivity | N/A |
| | functional | ClueGO, KEGG, QuickGO |
| | enrichment or | |
| | pathway | |
| | enrichment | |

### REFERENCE

1) CHANG, T. P., YU, S. L., LIN, S. Y., HSIAO, Y. J., CHANG, G. C., YANG, P. C. & CHEN, J. J. 2010. Tumor suppressor HLJ1 binds and functionally alters nucleophosmin via activating enhancer binding protein 2alpha complex formation. Cancer Res, 70, 1656-67.
2) LI, Z., BOONE, D. & HANN, S. R. 2008. Nucleophosmin interacts directly with c-Myc and controls c-Myc-induced hyperproliferation and transformation. Proc Natl Acad Sci USA, 105, 18794-9.
3) LIU, Y., ZHANG, F., ZHANG, X. F., QI, L. S., YANG, L., GUO, H. & ZHANG, N. 2012. Expression of nucleophosmin/NPM1 correlates with migration and invasiveness of colon cancer cells. J Biomed Sci, 19, 53.
4) TSUI, K. H., JUANG, H. H., LEE, T. H., CHANG, P. L., CHEN, C. L. & YUNG, B. Y. 2008. Association of nucleophosmin/B23 with bladder cancer recurrence based on immunohistochemical assessment in clinical samples. Acta Pharmacol Sin, 29, 364-70.
5) CILLONI, D., MESSA, F., ROSSO, V., ARRUGA, F., DEFILIPPI, I., CARTURAN, S., CATALANO, R., PAUTASSO, M., PANUZZO, C., NICOLI, P., MESSA, E., MOROTTI, A., IACOBUCCI, I., MARTINELLI, G., BRACCO, E. & SAGLIO, G. 2008. Increase sensitivity to chemotherapeutical agents andcytoplasmatic interaction between NPM leukemic mutant and NF-kappaB in AML carrying NPM1 mutations. Leukemia, 22, 1234-40.
6) PIANTA, A., PUPPIN, C., PASSON, N., FRANZONI, A., ROMANELLO, M., TELL, G., DI LORETO, C., BULOTTA, S., RUSSO, D. & DAMANTE, G. 2011. Nucleophosmin delocalization in thyroid tumour cells. Endocr Pathol, 22, 18-23.
7) KOSTKA, D. & SPANG, R. 2004. Finding disease specific alterations in the coexpression of genes. Bioinformatics, 20 Suppl 1, i194-9.
8) WU, Y., LIU, F., LUO, S., YIN, X., HE, D., LIU, J., YUE, Z. & SONG, J. 2019. Coexpression of key gene modules and pathways of human breast cancer cell lines. Biosci Rep, 39.
9) WANG, F., CHAN, L. W., CHO, W. C., TANG, P., YU, J., SHYU, C. R., TSUI, N. B., WONG, S. C., SIU, P. M., YIP, S. P. & YUNG, B. Y. 2014a. Novel approach for coexpression analysis of E2F1-3 and MYC target genes in chronic myelogenous leukemia. Biomed Res Int, 2014, 439840.
10) WANG, F., WANG, B., LONG, J., WANG, F. & WU, P. 2019a. Identification of candidate target genes for endometrial cancer, such as ANO1, using weighted gene co-expression network analysis. Exp Ther Med, 17, 298-306.
11) WANG, G. X., CHO, K. W., UHM, M., HU, C. R., LI, S., COZACOV, Z., XU, A. E., CHENG, J. X., SALTIEL, A. R., LUMENG, C. N. & LIN, J. D. 2014b. Otopetrin 1 protects mice from obesity-associated metabolic dysfunction through attenuating adipose tissue inflammation. Diabetes, 63, 1340-52.
12) WANG, Q., MA, X., CHEN, Y., ZHANG, L., JIANG, M., LI, X., XIANG, R., MIAO, R., HAJJAR, D. P., DUAN, Y. & HAN, J. 2014c. Identification of interferon-y as a new molecular target of liver X receptor. Biochem J, 459, 345-54. WANG, Q., ROY, B. & DWIVEDI, Y. 2019b. Co-expression network modeling identifies key long non-coding RNA and mRNA modules in altering molecular 177 phenotype to develop stress-induced depression in rats. Transl Psychiatry, 9, 125.
13) WANG, W., JIANG, W., HOU, L., DUAN, H., WU, Y., XU, C., TAN, Q., LI, S. & ZHANG, D. 2017. Weighted gene co-expression network analysis of expression data of monozygotic twins identifies specific modules and hub genes related to BMI. BMC Genomics, 18, 872. WANG, X. & LIN, Y. 2008. Tumor necrosis factor and cancer, buddies or foes? Acta Pharmacol Sin, 29, 1275-88.
14) WANG, X., MICHIE, S. A., XU, B. & SUZUKI, Y. 2007. Importance of IFN-gammamediated expression of endothelial VCAM-1 on recruitment of CD8+ T cells into the brain during chronic infection with Toxoplasma gondii. J Interferon Cytokine Res, 27, 329-38.
15) WANG, Y., MURAKAMI, Y., YASUI, T., WAKANA, S., KIKUTANI, H., KINOSHITA, T. & MAEDA, Y. 2013. Significance of glycosylphosphatidylinositol-anchored protein enrichment in lipid rafts for the control of autoimmunity. J Biol Chem, 288, 25490-9.
16) WANG, Z., BAO, W., ZOU, X., TAN, P., CHEN, H., LAI, C., LIU, D., LUO, Z. & HUANG, M. 2019c. Co-expression analysis reveals dysregulated miRNAs and miRNA-mRNA interactions in the development of contrast-induced acute kidney injury. PLOS One, 14, e0218574.
17) RIQUELME MEDINA, I. & LUBOVAC-PILAV, Z. 2016. Gene Co-Expression Network Analysis for Identifying Modules and Functionally Enriched Pathways in Type 1 Diabetes. PLOS One, 11, e0156006.
18) TANG, R. & LIU, H. 2019. Identification of Temporal Characteristic Networks of Peripheral Blood Changes in Alzheimer's Disease Based on Weighted Gene Coexpression Network Analysis. Front Aging Neurosci, 11, 83.
19) SIWO, G. H., TAN, A., BUTTON-SIMONS, K. A., SAMARAKOON, U., CHECKLEY, L. A., PINAPATI, R. S. & FERDIG, M. T. 2015. Predicting functional and regulatory divergence of a drug resistance
20) LEE, T. I. & YOUNG, R. A. 2013. Transcriptional regulation and its misregulation in disease. Cell, 152, 1237-51.
21) MILLER, D. M., THOMAS, S. D., ISLAM, A., MUENCH, D. & SEDORIS, K. 2012a. c-Myc and cancer metabolism. Clin Cancer Res, 18, 5546-53.
22) FLYNT, A. S. & LAI, E. C. 2008. Biological principles of microRNA-mediated regulation: shared themes amid diversity. Nat Rev Genet, 9, 831-42.
23) LIN, S. & GREGORY, R. I. 2015. MicroRNA biogenesis pathways in cancer. Nat Rev Cancer, 15, 321-33.

What is claimed is:

1. A method for identifying a genetic trait of cells in a state of interest, said method comprises the steps of:
a. Obtaining a first gene expression data from cells in said state of interest;
b. Obtaining a second gene expression data from cells in a reference state;
c. Conducting one or both of the following steps:
1. Identifying a first set of target genes, wherein each gene in said first set of target genes is strongly co-expressed with another gene in said first set of target genes in said state of interest as compared to said reference state by:
i. Conducting a first co-expression analysis on said first gene expression data to arrive at a first co-expression data;
ii. Conducting a second co-expression analysis on said second gene expression data to arrive at a second co-expression data;
iii. Comparing said first and second co-expression data to identify said first set of target genes;
2. Identifying a second set of target genes, wherein each target gene in said second set of target genes are differentially expressed genes with high connectivity in said state of interest as compared to said reference state by:
i. Conducting differential expression analysis on said first gene expression data to identify a set of differentially expressed genes in said state of interest with respect to said reference state;
ii. Identify said second set of target genes with high connectivity among said set of differentially expressed genes;
d. Identifying a third set of target genes, wherein each target gene in said third set of target genes is strongly co-expressed with NPM1 in said state of interest as compared to said reference state;
e. Conducting functional enrichment or pathway enrichment on said target genes obtained from steps (c) to (d);
f. Identifying signaling pathways associated with said target genes; and
g. Comparing said signaling pathways against a database to identify said genetic trait.

2. The method of claim 1, wherein said state of interest is selected from the group consisting of breast cancer, ovarian cancer, lung cancer, colorectal cancer, small cell lung cancer, liver cancer and prostate cancer.

3. The method of claim 1, wherein said reference state is a healthy state or a state different from said state of interest.

4. The method of claim 1, wherein said genetic trait is selected from the group consisting of cancer reoccurrence, cancer chemoresistance, cancer staging, drug sensitivity, platinum drug resistance, cancer diagnosis, and metastatic cancer staging.

5. The method of claim 2, wherein said state of interest is liver cancer and said genetic trait is liver cancer development from HBV infection.

6. The method of claim 1, wherein said first or second co-expression analysis is selected from one or more of whole genome co-expression analysis, gene co-expression network analysis and weighted gene co-expression network analysis.

7. The method of claim 1, wherein said first gene expression data or said second gene expression data is:
a. obtained using Next Generation Sequencing, Openarray technology, qPCR or Microarray technology; or
b. retrieved from a data repository.

8. The method of claim 1, wherein said step (d) further comprises identifying one or more sets of target genes, wherein each target gene in said one or more sets of target genes is strongly co-expressed with a gene of interest in said state of interest as compared to said reference state.

9. The method of claim 8, wherein:
a. said gene of interest is selected from the group consisting of ERBB2, BRCA1, BRCA2, BARD1, BRIP1, PALB2, RAD51, RAD54L, XRCC3, ERBB2, ESR1, PGR, GATA3, PIK3CA, TP53, PPM1D, RB1CC1, HMMR, NQO2, SLC22A18, PTEN, EGFR, KIT, NOTCH1, NOTCH4, FZD7, LRP6, FGFR1, and CCND1 when said state of interest is breast cancer;
b. said gene of interest is selected from the group consisting of BRCA1, BRCA2, MSH2, MLH1, ERBB2, KRAS, AKT2, PIK3CA, MYC, TP53, CTNNB1, PRKN, OPCML, AKT1 and CDH1 when said state of interest is ovarian cancer;
c. said gene of interest is selected from the group consisting of ERBB1, TGFA, AREG, EREG, MLH1, MLH3, MSH2, MSH6, TGFBR2, APC, MSH3, POLD1, POLE, DCC, KRAS, GALNT12, SMAD7, SMAD4, SMAD2, BAX, AXIN2, BRAF, CCND1, CHEK2, CTNNB1, FLCN, PIK3CA, TP53, BUB1, BUB1B, AURKA, SERP2, EFEMP2, FBN1, SPARC, and LINC0219 when said state of interest is colorectal cancer;
d. said gene of interest is selected from the group consisting of ERBB1, MYC, BCL2, FHIT, TP53, RB1, PTEN, PPP2R1B, EML4-ALK, CD74-ROS1, SLC34A2-ROS1, KIF5B-RET, RARB, RASSF1, KRAS, FHIT, CDKN2A, TP53, MET, BRAF, PIK3CA, IRF1, and PPP2R1B when said state of interest is lung cancer;
e. said gene of interest is selected from the group consisting of BCR-ABL, MLL-AF4, E2A-PBX1, TEL-AML1, c-MYC, CRLF2, PAX5, NOTCH1, TAL1, TAL2, LYL1, MLL-ENL, HOX11, MYC, LMO2, HOX11L2, PICALM-MLLT10, PML-RARalpha, AML1-ETO, PLZF-RARalpha, FLT3, KIT, NRAS, KRAS, AML1, CEBPA, CBFB, CHIC2, DNMT3A, ETV6, GATA2, JAK2, LPP, MLLT10, NPM1, NUP214, PICALM, SH3GL1, TERT, BCR-ABL, MECOM, RUNX1, CDKN2A, TP53, RB1, Bcl-2, p53, ATM, Fas, Bcl-6, CyclinD1, p16/INK4A, Fas, KIT, FIPIL1-PDGFRA, BCR-PDGFRA, CBL, TET2, ASXL1, SRSF2, NRAS, KRAS, CBL, RUNX1, SF3B1, ZRSR2, U2AF1, DNMT3A, EZH2, TP53, NPM1, JAK2, FLT3, SETBP1, CSF3R, ETNK1, CEBPA, IDH2, PTPN11, ARHGAP26, NF1, PML-RARA, PLZF-RARA, NUMA1-RARA, CD19, CD22, CD79, CD2, CD3, CD5, and CD8 when said state of interest is leukemia;
f. said gene of interest is selected from the group consisting of TGFA, IGF2, IGF1R, TERT, FZD7, HGF, MET, MYC, RB1, CDKN2A, TGFBR2, TP53, PTEN, CTNNB1, AXIN1, KEAP1, NFE2L2, PIK3CA, ARID1A, ARID2, CASP8, and IGF2R when said state of interest is liver cancer; and
g. said gene of interest is selected from the group consisting of AR, CDKN1B, NKX3.1, PTEN, GSTP1, TMPRSS2-ERG, TMPRSS2-ETV1, TMPRSS2-ETV4, TMPRSS2-ETV5, SLC45A3-ETV1, SLC45A3-ELK4, DDX5-ETV4, MAD1L1, KLF6, MXI1, ZFHX3, BRCA2, BRCA1, ATM, CHEK2, PALB2, MSH2, and MSH6 when said state of interest is prostate cancer.

10. The method of claim 1, wherein connectivity of said second set of target genes with high connectivity is evaluated by one or more methods selected from the group consisting of STRING, Reactome, KEGG, PathCards, Geneck, Cytoscape-ClueGO.

11. The method of claim 1, wherein said database is a library of predetermined relationship between said signaling pathways and said genetic trait.

12. The method of claim 1, wherein significance of co-expression of said first set of target genes is determined using one or more of the methods selected from the group consisting of Pearson correlation coefficient, Pearson product-moment correlation coefficient, cosine-angle uncentered correlation, cosine correlation, (non parametric) Kendall rank correlation and Spearman correlation, coefficient of determination (the R-squared measure of goodness of fit), Lack-of-fit sum of squares, Reduced chi-square, Regression validation, Mallows's Cp criterion, Bayesian information criterion, Kolmogorov-Smirnov test, Cramér-von Mises criterion, Anderson-Darling test, Shapiro-Wilk test, Chi-squared test, Akaike information criterion, Hosmer-Lemeshow test, Kuiper's test, Kernelized Stein discrepancy, Zhang's ZK, ZC and ZA tests, Moran test, Density Based Empirical Likelihood Ratio tests and Two-sample Kolmogorov-Smirnov test.

13. The method of claim 1, wherein said step (f) further comprises analyzing transcription factors associated with said genes.

14. A computer-implemented method for identifying a genetic trait of cells in a state of interest, comprising the steps of:
a. Obtaining a first gene expression data from cells in said state of interest;
b. Obtaining a second gene expression data from cells in a reference state;
c. Conducting one or both of the following steps:
1. Identifying a first set of target genes, wherein each gene in said first set of target genes is strongly co-expressed with another gene in said first set of target genes in said state of interest as compared to said reference state by:
i. Conducting a first co-expression analysis on said first gene expression data to arrive at a first co-expression data;
ii. Conducting a second co-expression analysis on said second gene expression data to arrive at a second co-expression data;
iii. Comparing said first and second co-expression data to identify said first set of target genes;
2. Identifying a second set of target genes, wherein each target gene in said second set of target genes are differentially expressed genes with high connectivity in said state of interest as compared to said reference state by:
i. Conducting differential expression analysis on said first gene expression data to identify a set of differentially expressed genes in said state of interest with respect to said reference state;
ii. Identify said second set of target genes with high connectivity among said set of differentially expressed genes;
d. Identifying a third set of target genes, wherein each target gene in said third set of target genes is strongly co-expressed with NPM1 in said state of interest as compared to said reference state;
e. Conducting functional enrichment or pathway enrichment on said target genes obtained from steps (c) to (d);
f. Identifying signaling pathways associated with said target genes; and
g. Comparing said signaling pathways against a database to identify said genetic trait.

15. A non-transitory computer-readable medium having stored thereon program instructions that, upon execution by a computing device, cause the computing device to perform operations for identifying a genetic trait of cells in a state of interest, said operations comprises the steps of:
a. Obtaining a first gene expression data from cells in said state of interest;
b. Obtaining a second gene expression data from cells in a reference state;
c. Conducting one or both of the following steps:
1. Identifying a first set of target genes, wherein each gene in said first set of target genes is strongly co-expressed with another gene in said first set of target genes in said state of interest as compared to said reference state by:
i. Conducting a first co-expression analysis on said first gene expression data to arrive at a first co-expression data;
ii. Conducting a second co-expression analysis on said second gene expression data to arrive at a second co-expression data;
iii. Comparing said first and second co-expression data to identify said first set of target genes;
2. Identifying a second set of target genes, wherein each target gene in said second set of target genes are differentially expressed genes with high connectivity in said state of interest as compared to said reference state by:
i. Conducting differential expression analysis on said first gene expression data to identify a set of differentially expressed genes in said state of interest with respect to said reference state;
ii. Identify said second set of target genes with high connectivity among said set of differentially expressed genes;
d. Identifying a third set of target genes, wherein each target gene in said third set of target genes is strongly co-expressed with NPM1 in said state of interest as compared to said reference state;
e. Conducting functional enrichment or pathway enrichment on said target genes obtained from steps (c) to (d);
f. Identifying signaling pathways associated with said target genes; and
g. Comparing said signaling pathways against a database to identify said genetic trait.

16. A computing device comprising:
1) a processor;
2) memory; and
3) program instructions, stored in the memory, that upon execution by the processor cause the computing device to perform operations for identifying a genetic trait of cells in a state of interest, said operations comprises the steps of:
a. Obtaining a first gene expression data from cells in said state of interest;
b. Obtaining a second gene expression data from cells in a reference state;
c. Conducting one or both of the following steps:
1. Identifying a first set of target genes, wherein each gene in said first set of target genes is strongly co-expressed with another gene in said first set of target genes in said state of interest as compared to said reference state by:
i. Conducting a first co-expression analysis on said first gene expression data to arrive at a first co-expression data;
ii. Conducting a second co-expression analysis on said second gene expression data to arrive at a second co-expression data;
iii. Comparing said first and second co-expression data to identify said first set of target genes;
2. Identifying a second set of target genes, wherein each target gene in said second set of target genes are differentially expressed genes with high connectivity in said state of interest as compared to said reference state by:
i. Conducting differential expression analysis on said first gene expression data to identify a set of differentially expressed genes in said state of interest with respect to said reference state;
ii. Identify said second set of target genes with high connectivity among said set of differentially expressed genes;
d. Identifying a third set of target genes, wherein each target gene in said third set of target genes is strongly co-expressed with NPM1 in said state of interest as compared to said reference state;
e. Conducting functional enrichment or pathway enrichment on said target genes obtained from steps (c) to (d);
f. Identifying signaling pathways associated with said target genes; and
g. Comparing said signaling pathways against a database to identify said genetic trait.
